(19) United States
(12) Patent Application Publication
(10) Pub. No.: US 2025/0326714 A1
(43) Pub. Date: Oct. 23, 2025

(54) PURE FORMS OF CRYSTALLINE ATICAPRANT

(71) Applicant: Janssen Pharmaceuticals, Inc., Titusville, NJ (US)

(72) Inventors: Riccardo Surmont, Beerse (BE); Mark Schmidt, Antwerp (BE); Vanina Popova, Nijlen (BE); Adam Savitz, Greenwich, CT (US); Rama Melkote, Basking Ridge, NJ (US); Wayne C. Drevets, Rancho Santa Fe, CA (US); Srihari Gopal, Belle Mead, NJ (US); Darrel Pemberton, Oud Turnhout (BE); Chakradhar Lagishetty, King of Prussia, PA (US); Iva Kezic, Antwerp (BE)

(21) Appl. No.: 19/254,965

(22) Filed: Jun. 30, 2025

Related U.S. Application Data
(63) Continuation of application No. 18/179,025, filed on Mar. 6, 2023.
(60) Provisional application No. 63/317,483, filed on Mar. 7, 2022.

(51) Int. Cl. C07D 207/06; A61K 45/06; A61P 25/24

(52) U.S. Cl. CPC C07D 207/06; A61K 45/06; A61P 25/24; C07B 2200/13

(57) ABSTRACT

The disclosure provides compositions containing pure aticaprant, solvates of aticaprant, and methods of treating major depressive disorder using aticaprant.

### CROSS REFERENCE TO RELATED APPLICATIONS

This application is a continuation of U.S. patent application Ser. No. 18/179,025, filed Mar. 6, 2023, which claims the benefit of U.S. Provisional Patent Application No. 63/317,483, filed on Mar. 7, 2022, which disclosures are incorporated by reference herein.

### TECHNICAL FIELD

The present disclosure relates to chemically and/or enantiomerically pure aticaprant, such as pure crystalline Form III of aticaprant, compositions containing the same, and methods of using the same.

### BACKGROUND

Kappa opioid receptors (KOR) and their native ligand dynorphin are localized in areas of the brain that effect reward and stress and may play a key role in mood, stress, and addictive disorders. Chronic stress, substance abuse, and acute withdrawal lead to increased dynorphin expression, activating KORs and subsequent downstream signaling pathways to inhibit mesolimbic dopamine surge, contributing to negative affective states. The behavioral pharmacology of KOR antagonism has been tested in animal models of anhedonia, depression, and anxiety and found to have meaningful effects that may translate to therapeutic benefit in humans. KOR antagonists may be effective for the treatment of patients with mood disorders, perhaps by modulating the negative affective state associated with stress response.

Aticaprant is an effective treatment for patients diagnosed with depression. However, synthetic routes to aticaprant are plagued with difficulties, including safety concerns, low yielding, and low purities, particularly on a large scale.

What is needed are new compounds and treatments for patients having depression, and optionally anhedonia.

### SUMMARY

In some aspects, the disclosure provides a tetrahydrofuran solvate of aticaprant:

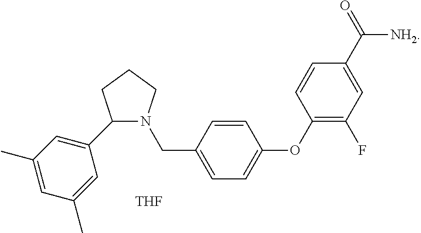 [SMILES: "THF" ; Cc1cc(C)cc(C2CCCN2Cc2ccc(Oc3ccc(C(N)=O)cc3F)cc2)c1]

In other aspects, the disclosure provides processes for preparing the tetrahydrofuran solvate of aticaprant.

In further aspects, the disclosure provides a tetrahydrofuran solvate of aticaprant prepared according to the processes described herein.

In yet other aspects, the disclosure provides compositions comprising a crystalline form of aticaprant and less than about 0.05% by weight, based on the weight of the composition, of 3,4-bis(4-((2-(3,5-dimethylphenyl) pyrrolidin-1-yl)methyl) phenoxy)benzamide:

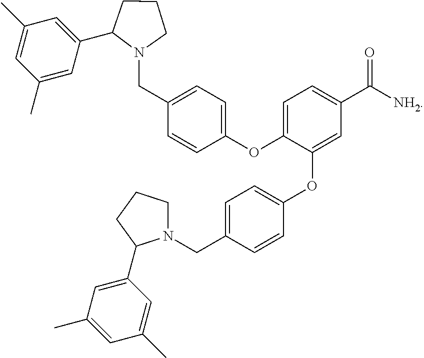 [SMILES: Cc1cc(C)cc(C2CCCN2Cc2ccc(Oc3ccc(C(N)=O)cc3Oc3ccc(CN4CCCC4c4cc(C)cc(C)c4)cc3)cc2)c1]

In still further aspects, the disclosure provides processes for preparing crystalline aticaprant:

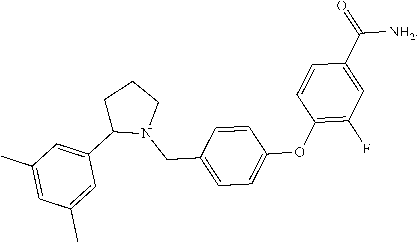 [SMILES: Cc1cc(C)cc(C2CCCN2Cc2ccc(Oc3ccc(C(N)=O)cc3F)cc2)c1]

In other aspects, the disclosure provides crystalline form of aticaprant prepared according to the processes described herein.

In further aspects, the disclosure provides methods of treating major depressive disorder in human patient with the composition or crystalline form of aticaprant described herein. In certain embodiments, the patient had a previous inadequate response to other antidepressant therapy.

In yet other aspects, the disclosure provides compositions or the crystalline form of aticaprant of claim 43 for use in treating major depressive disorder in a human patient, optionally in a patient having anhedonia. In certain embodiments, the patient had a previous inadequate response to other antidepressant therapy.

In still further aspects, the disclosure provides uses of the compositions or the crystalline form of aticaprant described herein for treating major depressive disorder in a human patient having anhedonia. In certain embodiments, the patient had a previous inadequate response to other antidepressant therapy.

### BRIEF DESCRIPTION OF THE DRAWINGS

FIG. 1 is the x-ray powder diffraction (XRPD) pattern of polymorph Form III of aticaprant (transmission mode).

FIG. 2 is the differential scanning calorimetry (DSC) thermogram of polymorph Form III of aticaprant.

FIG. 3 is the mDSC thermogram of polymorph Form III of aticaprant.

FIG. 4 is the trial design of Example 5.

FIG. 5 is a line graph showing the MADRS (Montgomery-Åsberg Depression Rating Scale) total score: least squares mean changes from baseline (±SE) during the treatment period for the enriched intent-to-treat (eITT) analysis set.

FIG. 6 is a plot showing MADRS total score changes at treatment week 6 for enriched and full population: MMRM results—estimated LS means and comparison versus placebo.

FIG. 7 is a line graph showing SHAPS (Snaith-Hamilton Pleasure Scale) total score: least squares mean changes from baseline (±SE) during the treatment period for the eITT analysis set.

FIG. 8 is a plot showing SHAPS total score changes at treatment week 6 for enriched and full population: MMRM (Mixed-effects Model for Repeated Measures) Results—estimated LSMeans and comparison versus placebo

FIG. 9 is a line graph showing MADRS total score: mean values (±SE) over time for the eITT analysis set.

FIG. 10-A is a line graph showing MADRS total score: mean values (±SE) over time for the full intent-to-treat (fITT) analysis set. FIG. 10-B is an excerpt from FIG. 10-A for treatment weeks 0-6.

FIG. 11 is a line graph showing MADRS total score: percentage of subjects with remission of depressive symptoms (total score ≤10) during the treatment period for the eITT analysis set.

FIG. 12 is a line graph showing MADRS total score: percentage of subjects with remission of depressive symptoms (total score ≤10) during the treatment period for the fITT analysis set.

FIG. 13 is a line graph showing MADRS total score: percentage of responders (≥30% improvement from baseline) during the treatment period for the eITT analysis set.

FIG. 14 is a line graph showing MADRS total score: percentage of responders (≥30% improvement from baseline) during the treatment period for the fITT analysis set.

FIG. 15 is a line graph showing MADRS total score: percentage of responders (≥50% improvement from baseline) during the treatment period for the eITT analysis set.

FIG. 16 is a line graph showing MADRS total score: percentage of responders (≥50% improvement from baseline) during the treatment period for the fITT analysis set.

FIG. 17 is a line graph showing SHAPS total score: mean values (±SE) over time for the eITT analysis set.

FIG. 18 is a line graph showing SHAPS total score: mean values (±SE) over time for the fITT analysis set.

FIG. 19 illustrates the MADRS change from baseline by anhedonia severity.

FIG. 20-A is a line graph showing MADRS change from baseline for patients with high anhedonia, i.e., SHAPS≥38. FIG. 20-B is a line graph showing MADRS change from baseline for patients with low anhedonia, i.e., SHAPS<38.

FIG. 21 is bar graph showing the comparison of MADRS in patients having low and high anhedonia.

FIG. 22 is a line graph showing the ASEX total score mean change from baseline.

FIG. 23 is a bar graph showing ASEX item level change total score mean change from baseline.

FIG. 24 is a bar graph showing the SHAPS items: LS means for change from baseline at week 6 by baseline SHAPS total score for the fITT analysis set. In this figure and going from top to bottom, the bars alternatively refer to placebo or aticaprant. For example, the first bar refers to aticaprant, the second bar refers to placebo, the third bar refers to aticaprant, etc.

FIG. 25 is a plot showing MADRS total score: difference of LSMeans (60% at Weeks 6 by different subgroups for the fITT analysis set. In this plot, <17 indicates mild severity; 18-24 indicates mild to moderate severity, and 25-30 indicates moderate to severe.

### DETAILED DESCRIPTION OF ILLUSTRATIVE EMBODIMENTS

All individual features (e.g., particular embodiments or specific preferred features) mentioned herein may be taken in isolation or in combination with any other feature (including particular embodiment or preferred feature) mentioned herein; hence, preferred features may be taken in conjunction with other preferred features, or independently of them (and likewise with particular embodiments).

The disclosure provides compositions comprising chemically and/or enantiomerically pure crystalline Form of III aticaprant that are anhydrous and stable in the solid form.

The term “crystalline” refers to a solid form of a chemical moiety that contains a highly ordered intermolecular structure.

The term “polymorph” refers to a crystalline form of a molecule having one specific crystal structure. A crystalline compound may have one crystal form or may have two or more crystal forms, i.e., polymorphs. As is understood to those skilled in the art, polymorphs of a chemical compound may distinguished from each other by compared physicochemical properties such as solubility, dissolution rate, stability, bioavailability, among others. Polymorphs also may have different spectra selected from, without limitation, x-ray powder diffraction (XRPD), single crystal x-ray diffraction, thermogravimetric analysis (TGA), infrared spectroscopy, Raman spectroscopy, solid state nuclear magnetic resonance (NMR), differential scanning calorimetry (DSC), polarized light microscopy (PLM), hot stage microscopy, or dynamic solvent sorption.

The term “crystalline” refers to solid state form of a chemical moiety wherein the atoms, molecules, or ions are assembled in a highly ordered structure that extends in all directions. Thus, “crystalline” includes all crystalline forms of Compound I, including salts thereof. Characterization of crystalline forms may be performed by those skilled in the art including, without limitation, XRPD or DSC. Typically, the XRPD pattern contains sharp intensity peaks. This contrasts to the XRPD pattern of an amorphous form that often contains a broad, peak, without no identifying peaks. A crystalline form may be completely crystalline or partially crystalline. In some aspects, a crystalline sample may be 100% w/w crystalline. A crystalline sample may also contain solids that are amorphous. In certain aspects, a crystalline form may contain solids such that the sample is at least about 99% w/w crystalline, at least about 95% w/w amorphous, at least about 90% w/w crystalline, at least about 85% w/w crystalline, at least about 80% w/w crystalline, or the like.

The term “anhydrous” or “anhydrate” as used herein refers to a crystalline as described herein that substantially lacks water. In some aspects, an anhydrous form contains less than about 1% w/w of water. In other aspects, an anhydrous form contains less than about 0.9%, about 0.8%, about 0.7%, about 0.6%, about 0.5%, about 0.4%, about 0.3%, about 0.2%, about 0.1% w/w of water.

As provided herein, all temperature values may vary. Such variations may depend on instrument type, instrument parameters, laboratory techniques, and/or laboratory conditions. Unless otherwise defined, a recited temperature may vary. In some aspects, the temperatures noted herein vary by about 0.1°, about 0.5°, about 1°, about 2°, about 3°, about 4°, or about 5°.

Similarly, 2θ values obtained from the XRPD patterns also may vary. Such variations may depend on instrument type, instrument parameters, laboratory techniques, sample (including particle size, impurities, etc.), and/or laboratory conditions. Unless otherwise defined, the XRPD patterns and/or the 2θ peak values may vary. In certain aspects, the 2θ peak values vary (higher or lower) by about 0.05°, about 0.1°, about 0.15°, or about 0.2°. In other aspects, one or more of the 2θ peak values are higher by about 0.05°, about 0.1°, about 0.15°, or about 0.2°. In further aspects, one or more of the 2θ peak values are lower by about 0.05°, about 0.1°, about 0.15°, or about 0.2°.

As used herein, the term “corresponds to” may be used in reference to certain spectra. Thus, “corresponds to” includes a spectrum that is identical or substantially similar to another spectrum. One skilled in the art would be able to compare such spectra and determine if a spectrum corresponds to another. Thus, the term “corresponds to” is used herein to compare XRPD patterns, DSC thermograms, among others. In some aspects, one XRPD pattern corresponds to another XRPD pattern when their 20 values are within the margin of error as described above. In other aspects, one XRPD pattern corresponds to another XRPD pattern when the peaks have the same 2θ peak value, but one or more peaks have a different height (intensity). In further aspects, one XRPD pattern corresponds to another XRPD pattern when the peaks have the same 2θ peak value, but one or more peaks have a different peak area. In yet other aspects, one XRPD pattern corresponds to another XRPD pattern when the peaks have the same 2θ peak value, but one or more peak is obscured. Such obscured peaks may be due to impurities, excipients, or the like. Such obscured peaks typically do not prevent characterization of the crystalline form.

As used herein, “chemical purity” refers to the relative percentage of aticaprant in sample compared to one or more chemical compounds that are not aticaprant. The content or degree of chemical purity is determined in % in reference to the theoretical composition, thus, as used herein, “chemically pure” means the compound is substantially devoid of impurities. In some embodiments, pure or chemically pure aticaprant has a purity of at least about 99.7 to about 100% as measured by HPLC area %. In further embodiments, pure or chemically pure aticaprant has a purity of about 99.7 to about 100% as measured by HPLC area %. In other embodiments, pure or chemically pure aticaprant has a purity of about 99.7, about 99.8, about 99.9, about 99.95 or about 100% as measured by HPLC area %.

The term “optical purity” is a comparison of the optical rotation of a pure sample of aticaprant with unknown stereochemistry versus the optical rotation of a sample of pure aticaprant and is expressed as a percentage. “Enantiomerically pure” means that a compound, e.g., aticaprant, has an optical purity of at least about 99.5% as measured by HPLC area %. Preferably, the enantiomerically pure aticaprant contains at least about 99.5%, as measured by HPLC area %, of S-aticaprant. In other embodiments, enantiomerically pure aticaprant has an optical purity of about 99.5 to 99.9% or about 99.95 to 99.9% as measured by HPLC area %. In further embodiments, enantiomerically pure aticaprant has an optical purity of at least about 99.9% or at least about 99.95%, as measured by HPLC area %.

The pure aticaprant, or compositions containing pure aticaprant, may be prepared using a novel intermediate. This novel intermediate is a tetrahydrofuran solvate of aticaprant:

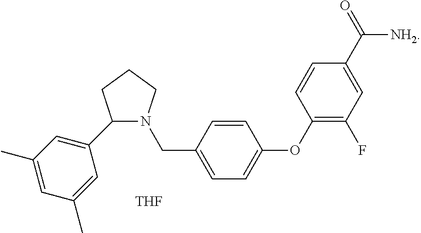 [SMILES: "THF" ; Cc1cc(C)cc(C2CCCN2Cc2ccc(Oc3ccc(C(N)=O)cc3F)cc2)c1]

In some embodiments, the tetrahydrofuran solvate is the tetrahydrofuran solvate of S-aticaprant:

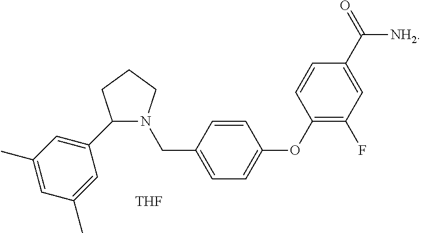 [SMILES: "THF" ; Cc1cc(C)cc(C2CCCN2Cc2ccc(Oc3ccc(C(N)=O)cc3F)cc2)c1]

Compositions comprising the tetrahydrofuran solvate also are described. Advantageously, compositions containing the tetrahydrofuran solvate of aticaprant contain fewer impurities than compositions containing other intermediates leading to the production of aticaprant. In particular, compositions containing the tetrahydrofuran solvate of aticaprant contain about 0.10% by weight or less, based on the weight of the composition, of 3-fluoro-4-(4-formylphenoxy)benzamide:

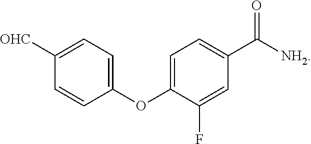 [SMILES: NC(=O)c1ccc(Oc2ccc(C=O)cc2)c(F)c1]

In some embodiments, compositions containing the tetrahydrofuran solvate of aticaprant contain about 0.1, about 0.09, about 0.08, about 0.07, about 0.06, about 0.05, about 0.04, about 0.03, about 0.02, about 0.01, about 0.009, about 0.008, about 0.007, about 0.006, about 0.005, about 0.004, about 0.003, about 0.002, or about 0.001% by weight, based on the weight of the composition, or less of 3-fluoro-4-(4-formylphenoxy)benzamide. In other embodiments, compositions containing the tetrahydrofuran solvate of aticaprant contain about 0.001 to about 0.1, about 0.001 to about 0.05, about 0.001 to about 0.01, about 0.001 to about 0.005, about 0.005 to about 0.1, about 0.005 to about 0.05, about 0.005 to about 0.01, about 0.01 to about 0.1, about 0.01 to about 0.05, or about 0.05 to about 0.1% by weight, based on the weight of the composition, of 3-fluoro-4-(4-formylphenoxy)benzamide.

Compositions containing the tetrahydrofuran solvate of aticaprant may contain at least about 99.5% by weight, based on the weight of the composition, of the tetrahydrofuran solvate of aticaprant. In some embodiments, compositions containing the tetrahydrofuran solvate of aticaprant contain at least about 99.5, about 99.6, about 99.7, about 99.8, about 99.9, about 99.95, or about 99.99% by weight, based on the weight of the composition, of the tetrahydrofuran solvate of aticaprant.

The tetrahydrofuran solvate of aticaprant, or compositions containing the same, also may contain less than 0.05% by weight, based on the total weight of the composition, of one or more of an organic impurity, inorganic impurity, or residual solvent. Examples of organic impurities include, without limitation, starting materials, by-products, intermediates, degradation products, reagents, ligands, catalysts, or combinations thereof. Examples of inorganic impurities include, without limitation, reagents, ligands, catalysts, heavy metals, inorganic salts, or other materials such as filter aids, charcoal, sulfated ash, among others. In some embodiments, the residual solvent is one or more of tetrahydrofuran, ethanol, and water. In further embodiments, the residual solvent is tetrahydrofuran. In further embodiments, the residue solvent is one or more of acetic acid, acetone, isobutyl acetate, anisole, isopropyl acetate, 1-butanol, methyl acetate, 2-butanol, 3-methyl-1-butanol, butyl acetate, methylethyl ketone, tert-butylmethyl ether, 2-methyl-1-propanol, dimethyl sulfoxide, pentane, ethyl acetate, 1-pentanol, ethyl ether, 1-propanol, ethyl formate, 2-propanol, formic acid, propyl acetate, or triethylamine.

The term “intermediate” as used herein refers to a material produced during steps of the synthesis of the tetrahydrofuran solvate of aticaprant or crystalline aticaprant described herein, undergoes further chemical transformation

The term “ligand” as used herein refers to an agent with a strong affinity to a metal ion.

The term “solvent” as used herein refers to an inorganic or an organic liquid used as a vehicle for the preparation of solutions or suspensions in the processes described herein.

The term “starting material” as used herein refers to a material used in the synthesis of the tetrahydrofuran solvate of aticaprant or crystalline aticaprant prepared as described herein that is incorporated as an element into the structure of an intermediate and/or of the tetrahydrofuran solvate of aticaprant or crystalline aticaprant prepared as described herein.

In some aspects, compositions containing the tetrahydrofuran solvate contain about 0.10% by weight or less, based on the weight of the composition, of the tetrahydrofuran solvate R-aticaprant:

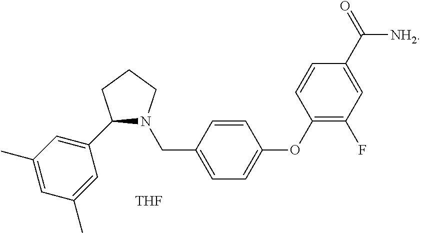 [SMILES: "THF" ; Cc1cc(C)cc([C@H]2CCCN2Cc2ccc(Oc3ccc(C(N)=O)cc3F)cc2)c1]

In some embodiments, compositions containing the tetrahydrofuran solvate of aticaprant contain about 0.1, about 0.09, about 0.08, about 0.07, about 0.06, about 0.05, about 0.04, about 0.03, about 0.02, about 0.01, about 0.009, about 0.008, about 0.007, about 0.006, about 0.005, about 0.004, about 0.003, about 0.002, or about 0.001% by weight, based on the weight of the composition, or less of the tetrahydrofuran solvate of R-aticaprant. In other embodiments, compositions containing the tetrahydrofuran solvate of aticaprant contain about 0.001 to about 0.1, about 0.001 to about 0.05, about 0.001 to about 0.01, about 0.001 to about 0.005, about 0.005 to about 0.1, about 0.005 to about 0.05, about 0.005 to about 0.01, about 0.01 to about 0.1, about 0.01 to about 0.05, or about 0.05 to about 0.1% by weight, based on the weight of the composition, of the tetrahydrofuran solvate of R-aticaprant.

In other aspects, compositions containing the tetrahydrofuran solvate contain about 0.10% by weight or less, based on the weight of the composition, of R-aticaprant:

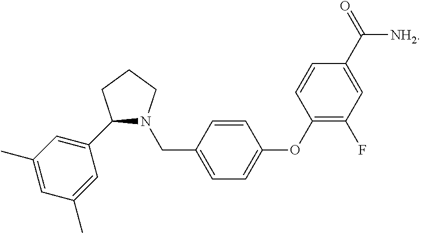 [SMILES: Cc1cc(C)cc([C@H]2CCCN2Cc2ccc(Oc3ccc(C(N)=O)cc3F)cc2)c1]

In some embodiments, compositions containing the tetrahydrofuran solvate of aticaprant contain about 0.1, about 0.09, about 0.08, about 0.07, about 0.06, about 0.05, about 0.04, about 0.03, about 0.02, about 0.01, about 0.009, about 0.008, about 0.007, about 0.006, about 0.005, about 0.004, about 0.003, about 0.002, or about 0.001% by weight, based on the weight of the composition, or less of R-aticaprant. In other embodiments, compositions containing the tetrahydrofuran solvate of aticaprant contain about 0.001 to about 0.1, about 0.001 to about 0.05, about 0.001 to about 0.01, about 0.001 to about 0.005, about 0.005 to about 0.1, about 0.005 to about 0.05, about 0.005 to about 0.01, about 0.01 to about 0.1, about 0.01 to about 0.05, or about 0.05 to about 0.1% by weight, based on the weight of the composition, of R-aticaprant.

The tetrahydrofuran solvate of aticaprant may be prepared by crystallizing aticaprant using tetrahydrofuran, an alcohol, and water. In some embodiments, the alcohol is ethanol or methanol. In other embodiments, the alcohol is ethanol. In further embodiments, the alcohol is methanol. Thus, in some embodiments, the processes for preparing the tetrahydrofuran solvate of aticaprant include reacting (2S)-2-(3,5-dimethylphenyl) pyrrolidine D-tartrate

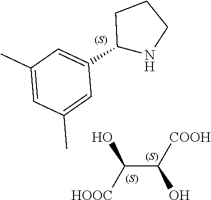

with 3-fluoro-4-(4-formylphenoxy)benzamide

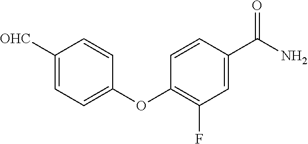 [SMILES: NC(=O)c1ccc(Oc2ccc(C=O)cc2)c(F)c1]

in the presence of a solvent. In some embodiments, the solvent is ethyl acetate, tetrahydrofuran, and 2-methyltetrahydrofuran, or a mixture thereof. In other embodiments, the solvent is tetrahydrofuran. In further embodiments, the solvent is ethyl acetate. In yet other embodiments, the solvent is 2-methyl-tetrahydrofuran. The processes for preparing the tetrahydrofuran solvate of aticaprant may be quenched using a base. Examples of suitable bases include, without limitation, an alkali hydroxide such as sodium hydroxide. The processes for preparing the tetrahydrofuran solvent of aticaprant also may be performed in the presence of a reducing agent. Examples of suitable reducing agents include sodium triacetoxyborohydride. In some embodiments, the reducing agent is sodium triacetoxyborohydride.

For processes that do not utilize tetrahydrofuran as the solvent, i.e., where the solvent is ethyl acetate or 2-methyl-tetrahydrofuran, the solvent is removed following completion of the reaction between (2S)-2-(3,5-dimethylphenyl) pyrrolidine D-tartrate and 3-fluoro-4-(4-formylphenoxy)benzamide. One of skill in the art would readily be able to apply suitable techniques to remove the solvent. For example, evaporation, distillation, among others may be used.

Once the solvent is removed, the resultant material, e.g., solid, is combined with tetrahydrofuran. In some embodiments, tetrahydrofuran is added to the material resulting from the reaction between (2S)-2-(3,5-dimethylphenyl) pyrrolidine D-tartrate and 3-fluoro-4-(4-formylphenoxy)benzamide. The amount of tetrahydrofuran utilized may depend on the scale of the reaction and may be determined by one skilled in the art.

In addition to tetrahydrofuran, an alcohol and water are added to the material resulting from the reaction between (2S)-2-(3,5-dimethylphenyl) pyrrolidine D-tartrate and 3-fluoro-4-(4-formylphenoxy)benzamide. In some embodiments, the alcohol is ethanol or methanol. In other embodiments, the alcohol is ethanol. In further embodiments, the alcohol is methanol. The amount of ethanol and water utilized may depend on the scale of the reaction, among others, and may be determined by one skilled in the art. In some embodiments, tetrahydrofuran is added to the solid material, followed by ethanol, and followed by water to form a tetrahydrofuran/ethanol/water solution. In other embodiments, tetrahydrofuran is added to the solid material, followed by water, and followed by ethanol to form a tetrahydrofuran/ethanol/water solution. In further embodiments, ethanol is added to the solid material, followed by tetrahydrofuran, and followed by water to form a tetrahydrofuran/ethanol/water solution. In yet other embodiments, ethanol is added to the solid material, followed by water, and followed by tetrahydrofuran to form a tetrahydrofuran/ethanol/water solution. In still further embodiments, water is added to the solid material, followed by ethanol, and followed by tetrahydrofuran to form a tetrahydrofuran/ethanol/water solution. In other embodiments, water is added to the solid material, followed by tetrahydrofuran, and followed by ethanol to form a tetrahydrofuran/ethanol/water solution.

The volume ratio of tetrahydrofuran to ethanol to water is about 1:1:1 to about 1:5:10. In some embodiments, the volume ration of tetrahydrofuran to ethanol to water is about 1:1:1, about 1:1:2, about 1:1:3, about 1:1:4, about 1:1:5, about 1:1:6, about 1:1:7, about 1:1:8, about 1:1:9, about 1:1:10, about 1:2:2, about 1:2:3, about 1:2:4, about 1:2:5, about 1:2:6, about 1:2:7, about 1:2:8, about 1:2:9, about 1:2:10, about 1:3:1, about 1:3:2, about 1:3:3, about 1:3:4, about 1:3:5, about 1:3:6, about 1:3:7, about 1:3:8, about 1:3:9, about 1:3:10, about 1:4:1, about 1:4:2, about 1:4:3, about 1:4:4, about 1:4:5, about 1:4:6, about 1:4:7, about 1:4:8, about 1:4:9, about 1:4:10, about 1:5:1, about 1:5:2, about 1:5:3, about 1:5:4, about 1:5:5, about 1:5:6, 1:5:7, about 1:5:8, about 1:5:9, or about 1:5:10. In other embodiments, the volume ratio of tetrahydrofuran to ethanol to water is about 1:1:2, based on the total volume of the solution. In further embodiments, the volume ratio of tetrahydrofuran to ethanol to water is about 1:1.6:3.2. In yet other embodiments, the volume ratio of tetrahydrofuran to ethanol to water is about 3:3:1. In still further embodiments, the volume ratio of tetrahydrofuran to ethanol to water is about 1:1:4. In other embodiments, the volume ratio of tetrahydrofuran to ethanol to water is about 4:5:10. In further embodiments, the volume ratio of tetrahydrofuran to ethanol to water is about 7:5:10. In yet other embodiments, the volume ratio of tetrahydrofuran to ethanol to water is about 3:5:10.

The tetrahydrofuran solvate of aticaprant is then crystallized from the tetrahydrofuran, ethanol, and water solution. One of skill in the art would be able to utilize suitable techniques for crystallizing the tetrahydrofuran solvate aticaprant. Examples of techniques include, without limitation, evaporating, cooling, concentrating, and seeding, or combinations thereof. In some embodiments, the tetrahydrofuran, ethanol, and water solution is concentrated. In further embodiments, the tetrahydrofuran, ethanol, and water solution is evaporated. In other embodiments, the tetrahydrofuran, ethanol, and water solution is cooled, e.g., to reduced temperatures. In further embodiments, the tetrahydrofuran, ethanol, and water solution is heated to elevated temperatures, e.g., the reflux temperature of the tetrahydrofuran, ethanol, and water solution, and then cooled to reduced temperatures. The reduced temperatures may be determined by one skilled in the art. In certain aspects, the reduced temperature is less than about room temperature, e.g., about 23° C. In further aspects, the reduced temperatures is less than about 20, about 15, about 10, or about 5° C.

Alternatively, or in addition to the above crystallization techniques, aticaprant tetrahydrofuran seed crystals may be added to the tetrahydrofuran, ethanol, and water solution. The term “seed crystals” as used herein refers to a solid sample of the tetrahydrofuran solvate of aticaprant that is present in a crystalline form. In some embodiments, the seed comprises comprise the tetrahydrofuran solvate of S-aticaprant. The seed crystals may be in the form of a variety of shapes including, without limitation, needles, blocks, or combinations thereof.

Contrary to other intermediates utilized to prepare aticaprant, the tetrahydrofuran solvate of aticaprant desirably is free from impurities or contains low levels of one or more impurities. In some embodiments, the tetrahydrofuran solvent of aticaprant, or compositions containing the, same contain low levels of 3-fluoro-4-(4-formylphenoxy)benzamide:

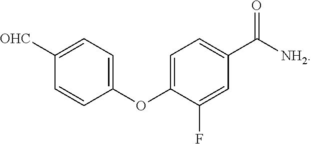 [SMILES: NC(=O)c1ccc(Oc2ccc(C=O)cc2)c(F)c1]

The term “low levels” as used herein to describe the amount of 3-fluoro-4-(4-formylphenoxy)benzamide in a sample of the tetrahydrofuran solvate of aticaprant. In some embodiments, the tetrahydrofuran solvate of aticaprant, or a composition containing the same, comprises about 0.10% by weight, based on the weight of the composition, of 3-fluoro-4-(4-formylphenoxy)benzamide. In other embodiments, the tetrahydrofuran solvate of aticaprant, or composition containing the same, contains about 0.1, about 0.09, about 0.08, about 0.07, about 0.06, about 0.05, about 0.04, about 0.03, about 0.02, about 0.01, about 0.009, about 0.008, about 0.007, about 0.006, about 0.005, about 0.004, about 0.003, about 0.002, or about 0.001% by weight, based on the weight of the composition, or less of 3-fluoro-4-(4-formylphenoxy)benzamide. In other embodiments, the tetrahydrofuran solvate of aticaprant, or compositions containing the tetrahydrofuran solvate of aticaprant, contain about 0.001 to about 0.1, about 0.001 to about 0.05, about 0.001 to about 0.01, about 0.001 to about 0.005, about 0.005 to about 0.1, about 0.005 to about 0.05, about 0.005 to about 0.01, about 0.01 to about 0.1, about 0.01 to about 0.05, or about 0.05 to about 0.1% by weight, based on the weight of the composition, of 3-fluoro-4-(4-formylphenoxy)benzamide.

The tetrahydrofuran solvate of aticaprant may be used to prepare crystalline forms of aticaprant. In some embodiments, the tetrahydrofuran solvate of aticaprant may be used to prepare chemically and/or enantiomerically pure S-aticaprant. In further embodiments, the tetrahydrofuran solvate may be used to prepare pure chemically and/or enantiomerically crystalline Form III of aticaprant. In other embodiments, the tetrahydrofuran solvate of aticaprant may be used to prepare pure chemically and/or enantiomerically crystalline Form III of S-aticaprant.

The chemically and/or enantiomerically pure forms of aticaprant, or compositions containing the same, prepared as described herein contain lower impurities than aticaprant forms in the art. In some embodiments, the chemically and/or enantiomerically form of aticaprant, or composition containing the same, described herein contains less than about 0.05% by weight, based on the weight of the composition, of 3,4-bis(4-((2-(3,5-dimethylphenyl) pyrrolidin-1-yl)methyl) phenoxy)benzamide:

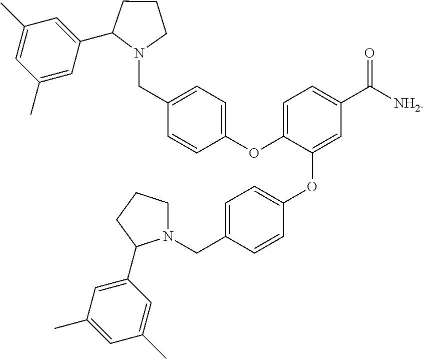 [SMILES: Cc1cc(C)cc(C2CCCN2Cc2ccc(Oc3ccc(C(N)=O)cc3Oc3ccc(CN4CCCC4c4cc(C)cc(C)c4)cc3)cc2)c1]

In other embodiments, the chemically and/or enantiomerically pure form of aticaprant, or composition containing the same, described herein contains less than about 0.05% by weight, based on the weight of the composition, of 3,4-bis(4-((2-(3,5-dimethylphenyl) pyrrolidin-1-yl)methyl) phenoxy)benzamide:

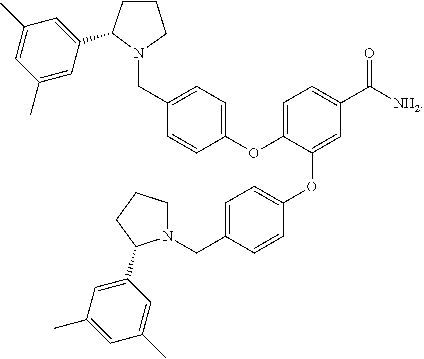 [SMILES: Cc1cc(C)cc([C@@H]2CCCN2Cc2ccc(Oc3ccc(C(N)=O)cc3Oc3ccc(CN4CCC[C@H]4c4cc(C)cc(C)c4)cc3)cc2)c1]

In some embodiments, the chemically and/or enantiomerically pure aticaprant, or compositions containing the same, prepared as described herein contains about 0.05, about 0.04, about 0.03, about 0.02, about 0.01, about 0.009, about 0.008, about 0.007, about 0.006, about 0.005, about 0.004, about 0.003, about 0.002, or about 0.001% by weight, based on the weight of the composition, or less of 3,4-bis(4-((2-(3,5-dimethylphenyl) pyrrolidin-1-yl)methyl) phenoxy)benzamide. In other embodiments, compositions containing the tetrahydrofuran solvate of aticaprant contain about 0.001 to about 0.05, about 0.001 to about 0.01, about 0.001 to about 0.005, about 0.005 to about 0.05, about 0.005 to about 0.01, or about 0.01 to about 0.05% by weight, based on the weight of the composition, of 3,4-bis(4-((2-(3,5-dimethylphenyl) pyrrolidin-1-yl)methyl) phenoxy)benzamide.

The chemically and/or enantiomerically pure aticaprant, or composition containing same, also contains about 0.10% by weight, based on the weight of the composition, or less of 3-fluoro-4-(4-formylphenoxy)benzamide. In other embodiments, the chemically and/or enantiomerically pure aticaprant, or composition containing the same, contains about 0.1, about 0.09, about 0.08, about 0.07, about 0.06, about 0.05, about 0.04, about 0.03, about 0.02, about 0.01, about 0.009, about 0.008, about 0.007, about 0.006, about 0.005, about 0.004, about 0.003, about 0.002, or about 0.001% by weight, based on the weight of the composition, or less of 3-fluoro-4-(4-formylphenoxy)benzamide. In other embodiments, the chemically and/or enantiomerically pure aticaprant, or composition containing the same, contains about 0.001 to about 0.1, about 0.001 to about 0.05, about 0.001 to about 0.01, about 0.001 to about 0.005, about 0.005 to about 0.1, about 0.005 to about 0.05, about 0.005 to about 0.01, about 0.01 to about 0.1, about 0.01 to about 0.05, or about 0.05 to about 0.1% by weight, based on the weight of the composition, of 3-fluoro-4-(4-formylphenoxy)benzamide.

The crystalline form of aticaprant, or compositions containing the same, may further contain about 0.10% by weight or less, based on the weight of the composition, of R-aticaprant:

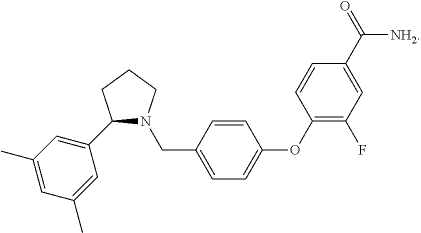 [SMILES: Cc1cc(C)cc([C@H]2CCCN2Cc2ccc(Oc3ccc(C(N)=O)cc3F)cc2)c1]

In some embodiments, compositions containing the crystalline form of aticaprant contain about 0.1, about 0.09, about 0.08, about 0.07, about 0.06, about 0.05, about 0.04, about 0.03, about 0.02, about 0.01, about 0.009, about 0.008, about 0.007, about 0.006, about 0.005, about 0.004, about 0.003, about 0.002, or about 0.001% by weight, based on the weight of the composition, or less of R-aticaprant. In other embodiments, compositions containing the crystalline form of aticaprant contain about 0.001 to about 0.1, about 0.001 to about 0.05, about 0.001 to about 0.01, about 0.001 to about 0.005, about 0.005 to about 0.1, about 0.005 to about 0.05, about 0.005 to about 0.01, about 0.01 to about 0.1, about 0.01 to about 0.05, or about 0.05 to about 0.1% by weight, based on the weight of the composition, of R-aticaprant.

Compositions containing aticaprant prepared as described herein may contain at least about 99.5% by weight, based on the weight of the composition, of chemically and/or enantiomerically pure aticaprant. In some embodiments, compositions containing aticaprant prepared as described herein contain at least about 99.5, about 99.6, about 99.7, about 99.8, about 99.9, about 99.95, or about 99.99% by weight, based on the weight of the composition, of chemically and/or enantiomerically pure aticaprant. In other embodiments, the compositions contain about 97 to about 100% by weight, based on the weight of the composition, of pure aticaprant. In further embodiments, compositions containing aticaprant prepared as described herein may contain at least about 99.5% by weight, based on the weight of the composition, of chemically and/or enantiomerically pure S-aticaprant. In yet other embodiments, compositions containing aticaprant prepared as described herein may contain at least about 99.5% by weight, based on the weight of the composition, of chemically and/or enantiomerically pure crystalline Form III of aticaprant. In still further embodiments, compositions containing aticaprant prepared as described herein may contain at least about 99.5% by weight, based on the weight of the composition, of chemically and/or enantiomerically pure crystalline Form III of S-aticaprant.

Crystalline aticaprant prepared as described herein, or compositions containing the same, also may contain less than 0.05% by weight, based on the total weight of the composition, of one or more of an organic impurity, inorganic impurity, or residual solvent. Examples of organic impurities include, without limitation, starting materials, by-products, intermediates, degradation products, reagents, ligands, catalysts, or combinations thereof. Examples of inorganic impurities include, without limitation, reagents, ligands, catalysts, heavy metals, inorganic salts, or other materials such as filter aids, charcoal, among others.

In general, the crystalline aticaprant prepared using the processes described herein, or compositions containing the same, contain about 0.15% by weight, based on the weight of the composition and administration of 2 g/day of aticaprant, or less of one or more impurities described herein to a patient. Alternatively, the crystalline aticaprant prepared using the processes described herein, or compositions containing the same, contain about 0.05% by weight, based on the weight of the composition and administration of greater than 2 g/day of aticaprant, or less of one or more impurities described herein to a patient. See, the thresholds described in ICH Harmonised Guideline, “Impurities: Guide for Residual Solvents Q3C(R8)”, Apr. 22, 2021, pages 1-44 and “Guidance for Industry—Q3A Impurities in New Drug Substances”, Revision 2, U.S. Department of Health and Human Services, June 2008, pages 1-14, which are incorporated by reference herein.

In some embodiments, the residual solvent is one or more of tetrahydrofuran, 2-methyl-tetrahydrofuran, ethanol, and water. In further embodiments, the residual solvent is tetrahydrofuran. In other embodiments, the residual solvent is ethanol. In yet further embodiments, the residual solvent is water. In still other embodiments, the residual solvent is 2-methyl-tetrahydrofuran. In further embodiments, the residual solvent is one or more of acetic acid, acetone, isobutyl acetate, anisole, isopropyl acetate, 1-butanol, methyl acetate, 2-butanol, 3-methyl-1-butanol, butyl acetate, methylethyl ketone, tert-butylmethyl ether, 2-methyl-1-propanol, dimethyl sulfoxide, pentane, ethyl acetate, 1-pentanol, ethyl ether, 1-propanol, ethyl formate, 2-propanol, formic acid, propyl acetate, or triethylamine. In other embodiments, the tetrahydrofuran solvate of aticaprant, or composition containing same, contains less than about 57 ppm of ethanol. In further embodiments, the tetrahydrofuran solvate of aticaprant, or composition containing same, contains less than about 200 ppm of 2-methyl-tetrahydrofuran.

Crystalline aticaprant as described herein may be prepared in processes comprising crystallizing the tetrahydrofuran solvate of aticaprant from 2-methyltetrahydrofuran and n-heptane. In some embodiments, processes provide crystalline S-aticaprant. In other embodiments, the processes provide crystalline Form III of aticaprant. In further embodiments, the processes provide crystalline Form III of S-aticaprant. Without wishing to be bound by theory, the inventors found that the use of n-heptane protected aticaprant from degradation, thereby resulting in higher chemical purities and increased yields.

The crystallization technique utilized to prepare chemically and/or enantiomerically pure aticaprant utilizes a temperature of less than about 48° C. In some embodiments, the crystallization temperature is less than about 45, about 40, about 35, about 30, about 25, about 20, about 15, about 10, about 5, about 0, about −5, about −10, about −15, about −20, about −25, about −30, about −35, or about −40° C. In other embodiments, the crystallization temperature is about −40 to about 40, about −40 to about 30, about −40 to about 20, about −40 to about 10, about −40 to about 0, about −40 to about −10, about −40 to about −20, about −40 to about −30, about −30 to about 40, about −30 to about 30, about −30 to about 20, about −30 to about 10, about −30 to about 0, about −30 to about −10, about −30 to about −20, about −20 to about 40, about −20 to about 30, about −20 to about 20, about −20 to about 10, about −20 to about 0, about −20 to about −10, about −10 to about 40, about −10 to about 30, about −10 to about 20, about −10 to about 10, about −10 to about 0, about 0 to about 40, about 0 to about 30, about 0 to about 20, about 0 to about 10, about 10 to about 40, about 10 to about 30, about 10 to about 20, about 20 to about 40, about 20 to about 30, or about 30 to about 40° C. Without wishing to be bound by theory, the inventors found that the lower temperature enabled a higher chemical purity for the aticaprant product.

The ratio of 2-methyltetrahydrofuran to n-heptane is about 1:1 to about 1:7. In some embodiments, the molar ratio of 2-methyltetrahydrofuran to n-heptane is about 1:1, about 1:2, about 1:3:about 1:4, about 1:5, about 1:6, or about 1:7. In other embodiments, the ratio of 2-methyltetrahydrofuran to n-heptane is about 1:1 to about 1:6, about 1:1 to about 1:5, about 1:1 to about 1:4, about 1:1 to about 1:3, about 1:1 to about 1:2, about 1:2 to about 1:7, about 1:2 to about 1:6, about 1:2 to about 1:5, about 1:2 to about 1:4, about 1:2 to about 1:3, about 1.3 to about 1:7, about 1:3 to about 1:6, about 1:3 to about 1:5, about 1:3 to about 1:4, about 1:4 to about 1:7, about 1:4 to about 1:6, about 1:4 to about 1:5, about 1:5 to about 1:7, about 1:5 to about 1:6, or about 1:6 to about 1:7. In further embodiments, the ratio of 2-methyltetrahydrofuran to n-heptane is about 1:3. In yet other embodiments, the ratio of 2-methyltetrahydrofuran to n-heptane is about 1:4. In still further embodiments, the ratio of 2-methyltetrahydrofuran to n-heptane is about 1:5. In other embodiments, the ratio of 2-methyltetrahydrofuran to n-heptane is about 1:6. In further embodiments, the ratio of 2-methyltetrahydrofuran to n-heptane is about 1:7.

In some aspects, the crystalline aticaprant, e.g., chemically and/or enantiomerically pure crystalline aticaprant, is crystalline Form III of aticaprant. Crystalline Form III of aticaprant may be characterized by a number of techniques including, without limitation, x-ray diffraction and differential scanning calorimetry. In some embodiments, crystalline Form III of aticaprant is characterized by x-ray diffraction. In other embodiments, crystalline Form III of aticaprant is characterized by four or more x-ray diffraction pattern peaks at 2θ (±0.2) of 4.1°, 9.0°, 17.6°, 18.0°, or 21.4°. In further embodiments, crystalline Form III of aticaprant is characterized by four or more x-ray diffraction pattern peaks at 2θ (±0.2) of 4.1°, 9.0°, 17.6°, 18.0°, or 21.4° and one or more additional peaks at 16.4°, 20.1°, 20.3°, 24.1°, and 25.7°. In yet other embodiments, crystalline Form III of aticaprant is characterized by four or more x-ray diffraction pattern peaks at 2θ (±0.2) of 4.1°, 9.0°, 17.6°, 18.0°, or 21.4° and one or more additional peaks at 15.1°, 16.4°, 20.0°, 20.1°, 20.3°, 24.1°, 25.0°, 25.7°, 26.2°, and 28.8°. In still further embodiments, crystalline Form III of aticaprant is characterized by four or more x-ray diffraction pattern peaks at 2θ (±0.2) of 4.1°, 9.0°, 17.6°, 18.0°, or 21.4° and one or more additional peaks at 8.2°, 9.7°, 12.0°, 13.5°, 15.1°, 16.4°, 19.4°, 28.4°, 20.0°, 20.1°, 20.3°, 24.1°, 25.0°, 25.7°, 26.2°, 28.8°, and 30.0°. In other embodiments, crystalline Form III of aticaprant is characterized by four or more x-ray diffraction pattern peaks at 2θ (±0.2) of 3.1°, 19.0°, 24.0°, 24.3°, or 26.2 and one or more additional peaks of Table 1.

TABLE 1

| Position (2θ) | Position (2θ) | Position (2θ) | Position (2θ) |
|---|---|---|---|
| 4.1 | 18.6 | 26.23 | 33.0 |
| 8.2 | 19.4 | 26.4 | 33.2 |
| 9.0 | 19.7 | 27.1 | 33.6 |
| 9.7 | 20.1 | 28.4 | 33.9 |
| 10.7 | 20.3 | 28.6 | 34.4 |
| 12.0 | 20.6 | 28.8 | 35.4 |
| 12.3 | 21.4 | 20.0 | 36.0 |
| 13.5 | 22.2 | 30.2 | 36.4 |
| 15.1 | 24.1 | 30.5 | 37.0 |
| 16.4 | 24.4 | 31.2 | 38.2 |
| 16.8 | 25.0 | 31.8 | 38.5 |
| 17.6 | 25.2 | 32.2 | 39.5 |
| 18.0 | 25.7 | 32.5 | |

In still other embodiments, crystalline Form III of aticaprant is characterized the x-ray diffraction pattern peaks in Table 2.

TABLE 2
Position (2θ)

4.1
8.2
9.0
9.7
12.0
13.5
15.1
16.4
17.6
18.0
19.4
19.7
20.1
20.3
21.4
24.1
25.0
25.7
26.3
28.4
28.8
30.0

In still other embodiments, crystalline Form III of aticaprant is characterized the x-ray diffraction pattern peaks in Table 3.

TABLE 3
Position (2θ)

4.1
8.2
9.0
9.7
10.7
12.0
12.3
13.5
15.1
16.4
16.8
17.6
18.0
18.6
19.4
19.7
20.1
20.3
20.6
21.4
22.2
24.1
24.4
25.0
25.2
25.7
26.3
26.4
27.1
28.4
28.6
28.8
30.0
30.2
30.5
31.2
31.8
32.2
32.5
33.0
33.2
33.6
33.9
34.4
35.4
36.0
36.4
37.0
38.2
38.5
39.5

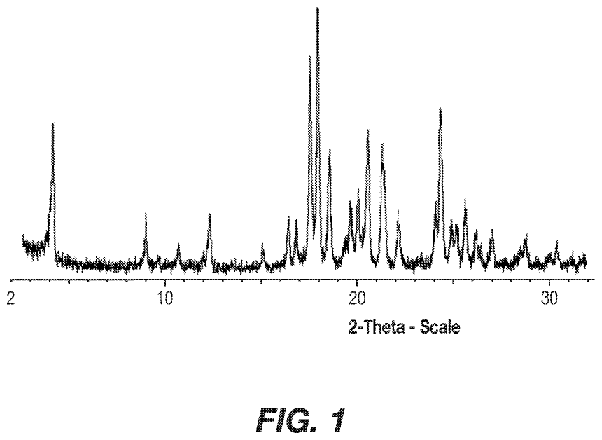

In further embodiments, crystalline Form III of aticaprant is characterized by an x-ray powder diffraction pattern that corresponds to FIG. 1.

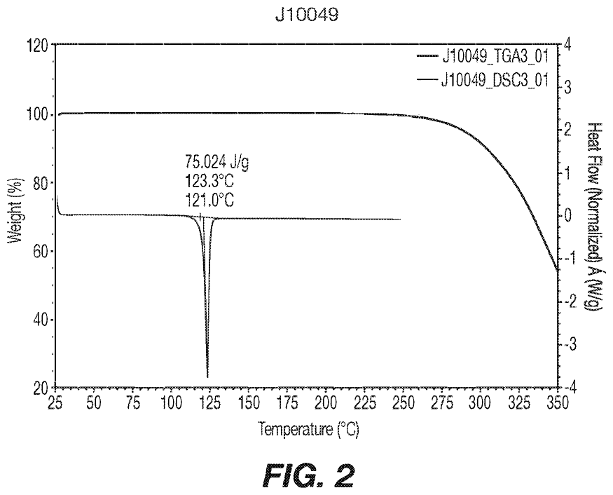

Crystalline Form III of aticaprant may also be characterized by differential scanning calorimetry. In some embodiments, the differential scanning calorimetry thermogram comprises a peak temperature (Tₘ) at about 121° C. In other embodiments, crystalline Form III of aticaprant is characterized by a differential scanning calorimetry thermogram that corresponds to FIG. 2.

### REFERENCE STANDARDS

The disclosure also provides reference standards containing aticaprant prepared as described herein. The term “reference standard” as used herein refers to aticaprant selected by FDA that an applicant seeking approval of an ANDA must use in conducting an in vivo bioequivalence study required for approval of the ANDA. See, e.g., “Referencing Approve Drug Products in ANDA Submissions-Guidance for Industry,” U.S. Department of Health and Human Services, pages 1-16, October 2020, which is incorporated by reference. In some embodiments, the reference standard contains a composition described herein. In further embodiments, the reference standard contains a crystalline form of aticaprant as described herein. Desirably, the reference standard comprises crystalline aticaprant having a chemical and/or enantiomeric purity of about 99.7%, as measured by HPLC.

### Treatment Methods

In one aspect of the present invention, methods are provided for treating patients having a more severe type of depression, i.e., major depressive disorder. In some embodiments, the patient also is experiencing moderate to severe anhedonia. Because MDD alone is difficult to treat, treatment patients having anhedonia are even more problematic since their ability to gauge pleasure is impaired. Thus, such patients often receive inadequate treatment due to ineffective medications, repeated and unnecessary medical appointments, lack of patient compliance, overall patient frustration, among others. Further, antidepressants are known to have a variety of side effects such as weight gain, metabolic side effects, extrapyramidal symptoms, akathisia, cognitive impairment, among others. Thus, patients may choose to refrain from or stop taking antidepressants to avoid or prevent any side-effects.

The methods described herein are effective in managing the patient's depression and anhedonia using the chemically and/or enantiomerically pure aticaprant described herein, e.g., chemically and/or enantiomerically pure crystalline Form III of aticaprant, or compositions containing the same. Desirably, the methods successfully permit the patient to manage their depression while simultaneously reducing anhedonia. In particular embodiments, the patients treated according to the described methods have moderate to severe anhedonia. The term “anhedonia” as used herein refers to the lack of or decreased ability to experience pleasure in daily activities. The term anhedonia includes loss of pleasure in sensory experiences (i.e., touch, taste, smell), as well as social interactions. In some embodiments, anhedonia and depressed mood are diagnostic criteria for a major depressive episode as part of MDD. Anhedonia also describes deficits in one or more components of reward-related behavior, also known as the pleasure cycle, such as wanting, liking, and learning. The pleasure cycle can be divided into three phases: the appetitive phase (dominated by wanting), the consummatory phase (dominated by liking), and the satiety phase (dominated by learning). The appetitive phase is characterized by the initial energy expenditure to attain a reward; the consummatory phase is enjoyment of the reward; and the satiety phase is characterized by learning and feedback integration.

To assess a potential effect on anhedonia, an anhedonia scale may be used. For example, the Snaith-Hamilton Pleasure Scale (SHAPS) analysis is a validated scale for the measurement of anhedonia. The SHAPS is a subject completed scale in which subjects score whether or not they experience pleasure in performing a list of activities or experiences. The SHAPS is a self-reported 14-item instrument, developed for the assessment of hedonic capacity. Subjects score whether they experience pleasure in performing a list of activities or experiences. Subjects can rate the answers as 1-4 where 1 indicates “Definitely agree”, 2 indicates “Agree”, 3 indicates “Disagree” and 4 indicates “Definitely disagree”. The subject's item responses are summed to provide a total score ranging from 14 to 56. A higher total SHAPS score indicates higher levels of current anhedonia. Physician/clinical judgment can be used to assess anhedonia separately or in conjunction with an anhedonia scale.

In some embodiments, the patient has moderate anhedonia. In other embodiments, the patient has severe anhedonia. An assessment of moderate or severe anhedonia is typically determined physician/clinical judgment and/or by one or more tests that provide insight into whether a patient has anhedonia. For example, the severity of the anhedonia may be determined using the SHAPS method. In some embodiments, a patient with moderate or severe anhedonia is considered to have a high level of anhedonia. For example, a patient with a SHAPS score of 38 or greater is considered to have moderate to severe anhedonia that can be considered a high level of anhedonia. In some embodiments, a high level of anhedonia is reflected by a SHAPS score of at least about 40, about 42, about 44, about 46, about 48, about 50, about 52, about 54, about 56, about 58, or higher. A patient with mild or no anhedonia would be considered to have a low level of anhedonia that is assessed by physician/clinical judgment and/or one or more tests. For example, a patient with a SHAPS score of less than 38 is considered to have low anhedonia. In certain embodiments, a patient with mild anhedonia may have a SHAPS score of 20 to less than 38, for example, a SHAPS score of 20 to about 36, about 22 to about 36, about 24 to about 36, about 26 to about 36, about 26 to about 34, about 26 to about 32, about 26 to about 30, about 26 to about 28, about 28 to about 36, about 28 to about 36, about 30, to about 36, about 32 to about 36, about 34 to about 36, about 20 to about 34, about 22 to about 34, about 24 to about 34, about 26 to about 32, about 26 to about 30, about 26 to about 28, about 28 to about 36, about 28 to about 34, about 28 to about 32, about 28 to about 30, about 30 to about 36, about 30 to about 34, about 30 to about 32, about 32 to about 36, about 32 to about 34, or about 34 to about 36. Typically, a SHAPS score of less than 20 can be considered to correspond to normal hedonic functioning, and for purposes of this disclosure, would fall into the low category of anhedonia, e.g., a SHAPS score of less than 38.

In some embodiments, the patient's anhedonia is reduced from a high level of anhedonia to a low level of anhedonia. In yet other embodiments, the patient's anhedonia is reduced by at least about 40%, as measured by the change from baseline in total score in an anhedonia scale following treatment with the chemically and/or enantiomerically pure aticaprant described herein, e.g., pure chemically and/or enantiomerically crystalline Form III of aticaprant, or compositions containing the same. In yet other embodiments, the patient's anhedonia is reduced by at least about 40%, about 50%, about 60%, about 70%, about 80%, about 90%, or about 95%, as measured by the change from baseline in total score in an anhedonia scale following treatment with the chemically and/or enantiomerically chemically and/or enantiomerically pure aticaprant described herein, e.g., pure crystalline Form III of aticaprant, or compositions containing the same. In still further embodiments, In yet other embodiments, the patient's anhedonia is reduced by about 40 to about 90%, about 50 to about 90%, about 60 to about 90%, about 70 to about 90%, about 80 to about 90%, about 40 to a bout 80%, about 50 to about 80%, about 60 to about 80%, about 70 to about 80%, about 40 to about 70%, about 50 to about 70%, about 60 to about 70%, about 40 to about 60%, about 50 to about 60%, or about 50 to about 60%, as measured by the change from baseline in total score in an anhedonia scale following treatment with the chemically and/or enantiomerically pure aticaprant described herein, e.g., chemically and/or enantiomerically pure crystalline Form III of aticaprant, or compositions containing the same. In other embodiments, the patient's anhedonia is ameliorated, i.e., reduced by 100%, as measured by the change from baseline in total score in an anhedonia scale following treatment with the chemically and/or enantiomerically pure aticaprant described herein, e.g., chemically and/or enantiomerically pure crystalline Form III of aticaprant, or compositions containing the same.

Reduction of anhedonia after initiating treatment with the pure aticaprant described herein, e.g., chemically and/or enantiomerically pure crystalline Form III of aticaprant, or compositions containing the same may be measured relative to the anhedonia of the patient as measured before treatment with the chemically and/or enantiomerically pure aticaprant described herein, e.g., chemically and/or enantiomerically pure crystalline Form III of aticaprant, or compositions containing the same, i.e., a baseline anhedonia measurement. In doing so, the treating clinician is able to calculate the change of anhedonia from the baseline to the real time anhedonia measurement at any point after treatment with the chemically and/or enantiomerically pure aticaprant described herein, e.g., chemically and/or enantiomerically pure crystalline Form III of aticaprant, or compositions containing the same. Thus, standard methods for measuring anhedonia may be used, such as an anhedonia scale, e.g., SHAPS.

Desirably, a baseline anhedonia measurement is obtained no more than about 1 week before initiating treatment with the chemically and/or enantiomerically pure aticaprant described herein, e.g., chemically and/or enantiomerically pure crystalline Form III of aticaprant, or compositions containing the same. In some embodiments, a baseline anhedonia measurement is obtained about 7 days, about 6 days, about 5 days, about 4 days, about 3 days, about 2 days, or about 1 day before treatment with the chemically and/or enantiomerically pure aticaprant described herein, e.g., chemically and/or enantiomerically pure crystalline Form III of aticaprant, or compositions containing the same. In further embodiments, a baseline anhedonia measurement is obtained about 24 hours, about 18 hours, about 12 hours, about 8 hours, about 4 hours, about 2 hours, about 1 hours, about 30 minutes, or about 15 minutes before initiating treatment with the pure aticaprant described herein, e.g., chemically and/or enantiomerically pure crystalline Form III of aticaprant, or compositions containing the same.

The patient's change of anhedonia will depend on several factors including, without limitation, anhedonia severity, patient's sensitivity to the chemically and/or enantiomerically pure aticaprant described herein, e.g., chemically and/or enantiomerically pure crystalline Form III of aticaprant, or compositions containing the same, other pharmaceutical agents being administered, among others. In some embodiments, the patient's anhedonia is reduced after about 3 weeks of treatment with the chemically and/or enantiomerically pure aticaprant described herein, e.g., chemically and/or enantiomerically pure crystalline Form III of aticaprant, or compositions containing the same. In other embodiments, the patient's anhedonia is reduced after about 3 weeks of treatment with the chemically and/or enantiomerically pure aticaprant described herein, e.g., chemically and/or enantiomerically pure crystalline Form III of aticaprant, or compositions containing the same. In further embodiments, the patient's anhedonia is reduced after about 3 weeks to about 6 weeks, and, in certain embodiments, through week 6, of treatment with the chemically and/or enantiomerically pure aticaprant described herein, e.g., chemically and/or enantiomerically pure crystalline Form III of aticaprant, or compositions containing the same. In certain embodiments, the patient's anhedonia is reduced by at least about 40%, as measured by the change from baseline in total score in an anhedonia scale following about 6 weeks of the treatment with v. In further embodiments, the anhedonia of the patient is reduced within about 3 weeks, and in some embodiments within about 3 weeks to about 6 weeks, as measured by the change from baseline in total score in an anhedonia scale and/or by physician/clinical judgement.

The methods described herein were found to not only improve the patient's depression and anhedonia symptoms, but resulted in fewer antidepressant side effects. Doing so resulted in less absenteeism (i.e., more visits or interactions with physicians), greater cognitive functioning, improvements in health-related quality of life, more interest and engagement in everyday activities, improvement in family and inter-personal relationships, ability to function in the workplace, fewer hospitalizations, among others.

As used herein, unless otherwise noted, the terms “subject” and “patient” refer to a human, who has been the object of treatment, observation or experiment. Preferably, the patient has experienced and/or exhibited at least one symptom of the disease or disorder to be treated and/or prevented. In some embodiments, the patient is an adult. As used herein, the term “adult” as used herein refers to a human that is about 18 years of age or older. In certain aspects, the patient is an elderly adult, i.e., greater than or equal to 65 years of age.

As used herein, unless otherwise noted, the terms “treating”, “treatment” and the like, shall include the management and care of a subject or patient (preferably mammal, more preferably human) for the purpose of combating a disease, condition, or disorder and includes the administration of a compound described herein to prevent the onset of the symptoms or complications, alleviate one or more of the symptoms or complications, or eliminate the disease, condition, or disorder.

As used herein, the term “depression” (also referred to as depressive disorder) includes major depressive disorder, persistent depressive disorder, seasonal affective disorder, postpartum depression, premenstrual dysphoric disorder, situational depression, anhedonia, melancholic, mid-life depression, late-life depression, bipolar depression, depression due to identifiable stressors, treatment resistant depression, or combinations thereof. In certain embodiments, the depression is major depressive disorder. In other embodiments, the major depressive disorder is with melancholic features or anxious distress. In further embodiments, the depression is treatment-resistant depression. In other embodiments, the depression is major depressive disorder with suicidal ideation.

As known in the art, a patient is considered to have major depressive disorder if exhibiting five or more symptoms during the same two week period that are a change from previous functioning; depressed mood and/or loss of interest/pleasure must be present; excluding symptoms clearly attributable to another medical condition. See, e.g., Table 4.

TABLE 4

| | |
|---|---|
| 1. | Depressed mood: Most of the day, nearly every day; may be subjective |
| | (e.g., feels sad, empty, hopeless) or observed by others (e.g., appears |
| | tearful); in children and adolescents, can be irritable mood |
| 2. | Loss of interest/pleasure: Markedly diminished interest/pleasure in |
| | all (or almost all) activities most of the day, nearly every day; may |
| | be subjective or observed by others |
| 3. | Weight loss or gain: Significant weight loss (without dieting) or |
| | gain (change of >5% body weight in a month), or decrease or increase in |
| | appetite nearly every day; in children, may be failure to gain weight as |
| | expected |
| 4. | Insomnia or hypersomnia: Nearly every day |
| 5. | Psychomotor agitation or retardation: Nearly every day and observable |
| | by others (not merely subjectively restless or slow) |
| 6. | Fatigue: Or loss of energy, nearly every day |
| 7. | Feeling worthless or excessive/inappropriate guilt: Nearly every day; |
| | guilt may be delusional; not merely self-reproach or guilt about being |
| | sick |
| 8. | Decreased concentration: Nearly every day; may be indecisiveness; may |
| | be subjective or observed by others |
| 9. | Thoughts of death/suicide” Recurrent thoughts of death (not just |
| | fear of dying), recurrent suicidal ideation without specific plan, or |
| | suicide attempt, or a specific plan for suicide |

In some embodiments, to be diagnosed with MDD, the following criteria also are met:

| | |
|---|---|
| 1. | Symptoms cause clinically significant distress or impairment |
| | in social, occupational, or other important areas of functioning |
| 2. | Episode not attributable to physiological effects of a substance or |
| | another medical condition |
| 3. | Episode not better explained by schizoaffective disorder, |
| | schizophrenia, schizophreniform disorder, delusional disorder, |
| | or other specified and unspecified schizophrenia spectrum |
| | and other psychotic disorders |
| 4. | No history of manic or hypomanic episode |

Major depressive disorder may be categorized as mild, moderate, or severe. In some embodiments, the MDD is mild. In other embodiments, the MDD is moderate. In further embodiments, the MDD is severe. As used herein, “mild MDD” applies to a patient having few, if any, symptoms in excess of those required to make the diagnosis, the intensity of the symptoms is distressing but manageable, and the symptoms result in minor impairment in social or occupational functioning. The mild MDD may be a single episode (ICD-10 F32.0) or a recurrent episode (ICD-10 F33.0). “Moderate MDD” applies to a patient having a number of symptoms, intensity of symptoms, and/or functional impairment are between those specified for “mild” and “severe.” The moderate MDD may be a single episode (ICD-10 F32.1) or a recurrent episode (ICD-10 F33.1). “Severe MDD” applies to a patient where the number of symptoms is substantially in excess of that required to make the diagnosis, the intensity of symptoms is seriously distressing and unmanageable, and the symptoms markedly interfere with social and occupational functioning, and urgent symptom control is necessary. In some embodiments, the severe MDD may be a single episode (ICD-10 F32.2) or a recurrent episode (ICD-10 F33.2). In other embodiments, MDD is classified according to the DSM-5 definition of Table 5.

TABLE 5
DSM-5 Criteria for MDD

| | |
|---|---|
| 1. Depressed Mood | At least 1 |
| 2. Loss of interest/pleasure (anhedonia) | |
| 1. Weight loss or gain | At least 5 |
| 2. Sleep problems | |
| 3. Psychomotor agitation or retardation | |
| 4. Guilt or worthlessness | |
| 5. Decreased concentration | |
| 6. Suicidality | |
| 7. Fatigue | |
| 1. Symptoms cause significant distress or impairment | Must have |
| 2. Not attributable to medical condition | all 4 |
| 3. Exclude schizophrenia disorders | |
| 4. No hx of mania or hypomania | |

Several scales are known in the art that may be utilized to diagnose or monitor patients with MDD. Examples of these scales include, without limitation, the Montgomery-Åsberg Depression Rating Scale (MADRS), Clinical Global Impression—Severity (CGI-S) scale, Symptoms of Major Depressive Disorder Scale (SMDDS), Self-Assessment of Treatment Experience (SATE) scale, and Massachusetts General Hospital (MGH) Antidepressant Treatment Response Questionnaire (ATRQ), i.e., MGH-ATRQ.

In some embodiments, MADRS is utilized to diagnose and/or monitor the patient. MADRS is a 10-item rating scale that is used in antidepressant studies. It is clinician-administered and designed to be used in subjects with MDD to measure the overall severity of depressive symptoms. The MADRS scale is validated, reliable, and acceptable to regulatory health authorities as a primary scale to determine efficacy in major depression. In some embodiments, MADRS is administered using the Structured Interview Guide for the MADRS (SIGMA). The scale consists of 10 items, each of which is scored from 0 (item not present or normal) to 6 (severe or continuous presence of the symptoms), summed for a total possible score of 60. Higher scores represent a more severe condition. The MADRS evaluates apparent sadness, reported sadness, inner tension, sleep appetite, concentration, lassitude, inability to feel (interest level), pessimistic thoughts, and suicidal thoughts.

In other embodiments, CGI-S is utilized to diagnose and/or monitor the patient's depression. CGI-S is a scale that rates the severity of the subject's illness at the time of assessment, relative to the clinician's past experience with subjects who have the same diagnosis and improvement with treatment. CGI-S provides an overall clinician-determined summary measure of severity of subject's illness that considers all available information, including knowledge of subject's history, psychosocial circumstances, symptoms, behavior, and impact of symptoms on subject's ability to function. CGI-S evaluates severity of psychopathology on scale of 0 to 7. Subject is assessed on severity of mental illness at time of rating according to: 0=not assessed; 1=normal (not at all ill); 2=borderline mentally ill; 3=mildly ill; 4=moderately ill; 5=markedly ill; 6=severely ill; 7=among most extremely ill patients.

In further embodiments, SMDDS is utilized to diagnose and/or monitor the patient's depression. SMDDS is a subjective rating of the patient. The SMDDS is a 16-item PRO measure. Each item is rated by the subject according to a 5-point Likert scale. Subjects respond to each question using a rating scale between 0 (“Not at all” or “Never”) to 4 (“Extremely” or “Always”). The total score ranges from 0 to 60. The SMDDS uses a 7-day recall period and verbal rating scales. Higher score indicates more severe depressive symptomatology.

In yet other embodiments, SATE is utilized to diagnose and/or monitor the patient's depression. SATE is a one to three questionnaire administered when the subject is unable to complete other evaluations, i.e., away from the clinical setting such as at home. SATE is useful to evaluate improvement or deterioration of depressive symptoms of the subjects over a short period of time. For rating overall depression, subject selected one option out of Improved, not changed or got worse; for depression improvement, subject selected one option out of slightly improved, much improved, very much improved and for depression worsen subject selected slightly worse, much worse, very much worse. See, Table 6.

TABLE 6
SATE Questionnaire

Question 1: Since starting this study medication, overall would you say
your depression is:
Improved
Got worse
Not changed
If the subject selects answer 1 (Improved), following question is asked:
Question 2: How much did your depression improve?
Slightly improved
Much improved
Very much improved
If the subject selects answer 3 (Got worse), following question is asked:
Question 3:How much did your depression worsen?
Slightly worse
Much worse
Very much worse

The MGH-ATRQ is a self-rated scale used to determine treatment resistance in patient's having MDD. This questionnaire examines the antidepressant treatment history, using specific anchor points to define the adequacy of both dose and duration of each antidepressant trial, and the degree of symptomatic improvement. The MGH-ATRQ permits determining treatment resistance in depression and is known to those skilled in the art.

In certain embodiments, the patient had an inadequate response to other antidepressant therapy. “Inadequate response” as used herein refers to a patient experiencing a less than about 50% reduction in depressive symptom severity from the start of initiating treatment. Typically, the inadequate response is during a current/active episode of the depression. In some embodiments, an inadequate response refers to a patient experiencing about 26 to less than about 50% reduction in depressive symptom severity from the start of initiating treatment. In other embodiments, an inadequate response refers to a patient experiencing about 26 to about 49, about 26 to about 45, about 26 to about 40, about 26 to about 35, about 26 to about 30, about 30 to about 49, about 30 to about 45, about 30 to about 40, about 30 to about 35, about 35 to about 49, about 35 to about 45, about 35 to about 40, about 40 to about 49, or about 40 to about 45% reduction in depressive symptom severity from the start of initiating treatment. A patient's response may be measured by one or more scales described herein and/or by physician/clinical judgment. In some embodiments, an inadequate response is measured by MGH-ATRQ, MADRS, or SHAPS. In further embodiments, an inadequate response is measured by MGH-ATRQ.

To the extent a patient is said to have a partial response to treatment, this refers to some minor to moderate symptomatic improvement since the initiation of treatment, but some of the initial symptoms are still present and troubling to the patient and these persistent symptoms still affect behavior and function. For instance, the patient's motivation, productivity, and interest in his or her usual activities may still be impaired.

The term “other antidepressant therapy” as used herein refers to an antidepressant medication or non-pharmacological treatment that is used to treat patients having depression. In some aspects, the other antidepressant therapy is an antidepressant medication. In other aspects, the other antidepressant therapy is a non-pharmacological treatment. In further aspects, the other antidepressant therapy is an antidepressant medication other than aticaprant.

The antidepressant medication is any pharmaceutical agent which can be used to treat depression. Suitable examples include, without limitation, mono-amine oxidase inhibitors, tricyclics, tetracyclics, non-cyclics, triazolopyridines, selective serotonin reuptake inhibitors (SSRI), serotonin receptor antagonists, serotonin noradrenergic reuptake inhibitors (SNRI), noradrenergic and specific serotonergic agents, noradrenaline reuptake inhibitors, or antipsychotics (typical or atypical antipsychotics). Examples of mono-amine oxidase inhibitors include phenelzine, tranylcypromine, moclobemide, and the like. Examples of tricyclics include imipramine, amitriptyline, desipramine, nortriptyline, doxepin, protriptyline, trimipramine, clomipramine, amoxapine, and the like. Examples of tetracyclics includes maprotiline, and the like. Examples of non-cyclics include nomifensine, and the like. Examples of triazolopyridines include trazodone, and the like. Examples of SSRIs include fluoxetine, sertraline, paroxetine, citalopram, citalopram, escitalopram, fluvoxamine, and the like. Examples of serotonin receptor antagonists include nefazadone, and the like. Examples of SNRIs include venlafaxine, milnacipran, desvenlafaxine, duloxetine, levomilnacipran and the like. Examples of noradrenergic and specific serotonergic agents include mirtazapine, and the like. Examples of noradrenaline reuptake inhibitors include reboxetine, edivoxetine and the like. Examples of typical antipsychotics include phenothiazines (e.g., chlorpromazine, thioridazine, fluphenazine, perphenazine, trifluoperazine, levomepromazine), thioxanthenes (e.g., thiothixene, flupentixol), butyrophenones (e.g., haloperidol), dibenzoxazepines (e.g., loxapine), dihydroindolones (e.g., molindone), substituted benzamides (e.g., sulpride, amisulpride), and the like. Examples of atypical antipsychotics include paliperidone, clozapine, risperidone, olanzapine, quetiapine, zotepine, ziprasidone, iloperidone, perospirone, blonanserin, sertindole, ORG-5222, sonepiprazole, aripiprazole, nemonapride, SR-31742, CX-516, SC-111, NE-100, divalproate (mood stabilizer) and the like. In further embodiments, the antidepressant medication includes natural products such as Kava-Kava, St. John's Wort, and the like or dietary supplements such as s-adenosylmethionine, and the like. In yet other embodiments, the antidepressant medication includes neuropeptides such as thyrotropin-releasing hormone and the like or compounds targeting neuropeptide receptors such as neurokinin receptor antagonists and the like. In still further embodiments, the antidepressant medication is a hormone such as triiodothyronine, and the like. In other embodiments, the antidepressant medication is SSRI, SNRI, or a combination thereof. Preferably, the antidepressant is a SSRI that is escitalopram, sertraline, paroxetine, fluoxetine or citalopram. In other embodiments, the antidepressant medication is a SNRI that is venlafaxine, duloxetine, vortioxetine or desvenlafaxine.

The non-pharmacologic treatment for use herein may be selected by one skilled in the art. In some embodiments, the non-pharmacologic treatment is psychotherapy, transcranial magnetic stimulation, or the like.

Therapeutically effective amounts/dosage levels and dosage regimens for the other antidepressant therapy may be readily determined by one of ordinary skill in the art. For example, therapeutic dosage amounts and regimens for pharmaceutical agents approved for sale are publicly available, for example as listed on packaging labels, in standard dosage guidelines, in standard dosage references such as the Physician's Desk Reference (Medical Economics Company or online at http:///www.pdrel.com) or other sources.

In some embodiments, other antidepressant therapy may include one antidepressant medication. In other embodiments, other antidepressant therapy includes two or more antidepressant medications. In further embodiments, other antidepressant therapy includes two antidepressant medications. In yet other embodiments, other antidepressant therapy includes three antidepressant medications. The attending physician would be able to select suitable antidepressant therapies for use as described herein.

In certain embodiments, the patient was receiving treatment with other antidepressant therapy prior to receiving the pure aticaprant described herein, e.g., pure crystalline Form III of aticaprant, or compositions containing the same. In some embodiments, the patient was receiving treatment with other antidepressant therapy that comprised a SSRI, SNRI, or a combination thereof. In other embodiments, the patient stopped treatment with other antidepressant therapy before initiating treatment with the pure aticaprant described herein, e.g., pure crystalline Form III of aticaprant, or compositions containing the same.

Also encompassed by the methods described herein include adjunctive treatment with an effective amount of one or more antidepressants. As used herein, the term “adjunctive treatment” and “adjunctive therapy” shall mean treatment of a patient in need thereof by administering the pure aticaprant described herein, e.g., pure crystalline Form III of aticaprant, or compositions containing the same in combination with one or more antidepressant(s), wherein the pure aticaprant described herein, e.g., pure crystalline Form III of aticaprant, or compositions containing the same and the antidepressant(s) are administered by any suitable means, simultaneously, sequentially, separately, or in a single pharmaceutical formulation.

In some aspects, the chemically and/or enantiomerically pure aticaprant described herein, e.g., chemically and/or enantiomerically pure crystalline Form III of aticaprant, or compositions containing the same is administered adjunctively with other antidepressant(s) currently being administered to the patient, including current antidepressant(s) to which the patient had an inadequate response, i.e., the antidepressant failed to treat the patient's depression. In other embodiments, the chemically and/or enantiomerically pure aticaprant described herein, e.g., chemically and/or enantiomerically pure crystalline Form III of aticaprant, or compositions containing the same is administered adjunctively with an antidepressant(s) not previously administered to the patient, i.e., a new antidepressant. In still other embodiments, the chemically and/or enantiomerically pure aticaprant described herein, e.g., chemically and/or enantiomerically pure crystalline Form III of aticaprant, or compositions containing the same is administered in a regimen with an antidepressant(s) previously administered to the patient.

Where the chemically and/or enantiomerically pure aticaprant described herein, e.g., chemically and/or enantiomerically pure crystalline Form III of aticaprant, or compositions containing the same and other antidepressant(s) are administered in separate dosage forms, the number of dosages administered per day for each active compound may be the same or different and more typically different. The antidepressant may be dosed as prescribed by the attending physician and/or by its label and the chemically and/or enantiomerically pure aticaprant described herein, e.g., chemically and/or enantiomerically pure crystalline Form III of aticaprant, or compositions containing the same is dosed as described herein. Typically, a patient is under concurrent treatment with both an antidepressant and the chemically and/or enantiomerically pure aticaprant described herein, e.g., chemically and/or enantiomerically pure crystalline Form III of aticaprant, or compositions containing the same, where both are administered by their prescribed dosing regimens. The chemically and/or enantiomerically pure aticaprant described herein, e.g., chemically and/or enantiomerically pure crystalline Form III of aticaprant, or compositions containing the same and antidepressant(s) may be administered according to simultaneous or alternating regimens, at the same or different times during the course of the therapy, concurrently in divided or single forms.

The chemically and/or enantiomerically pure aticaprant described herein, e.g., chemically and/or enantiomerically pure crystalline Form III of aticaprant, or compositions containing the same and the antidepressant(s) may be administered via the same or different routes of administration. Examples of suitable methods of administration include, but are not limited to, oral, intravenous (iv), intranasal (in) intramuscular (im), subcutaneous (sc), transdermal, buccal, or rectal. In some embodiments, the chemically and/or enantiomerically pure aticaprant, e.g., chemically and/or enantiomerically pure crystalline Form III of aticaprant, or compositions containing the same as described herein is administered orally.

Treatment with the chemically and/or enantiomerically pure aticaprant described herein, e.g., chemically and/or enantiomerically pure crystalline Form III of aticaprant, or compositions containing the same as described herein has several advantages over the treatments in the art. In some embodiments, the patient does not experience many of the side effects that are associated with other antidepressants, i.e., antidepressants other than the chemically and/or enantiomerically pure aticaprant described herein, e.g., chemically and/or enantiomerically pure crystalline Form III of aticaprant, or compositions containing the same. In certain aspects, the patient does not experience weight gain during the treatment with the chemically and/or enantiomerically pure aticaprant described herein, e.g., chemically and/or enantiomerically pure crystalline Form III of aticaprant, or compositions containing the same. As used herein, the term “weight gain” refers to an increase in the weight of patient, relative to the weight of the patient before taking the chemically and/or enantiomerically pure aticaprant described herein, e.g., chemically and/or enantiomerically pure crystalline Form III of aticaprant, or compositions containing the same or the weight of the patient that is assessed at the time of the initial administration of the chemically and/or enantiomerically pure aticaprant described herein, e.g., chemically and/or enantiomerically pure crystalline Form III of aticaprant, or compositions containing the same. In certain embodiments, the patient may actually see a decrease in overall weight, relative to the weight of the patient before taking the chemically and/or enantiomerically pure aticaprant described herein, e.g., chemically and/or enantiomerically pure crystalline Form III of aticaprant, or compositions containing the same. In further embodiments, the patient's weight is stable, i.e., does not increase or decrease. In certain embodiments, the patient does not experience a clinically relevant weight gain which is characterized as a weight increase of ≥7%.

This is contrary to many other antidepressants where weight gain, including clinically relevant weight gain, is a common, but unfortunate, side-effect.

In further aspects, the patient does not experience a decrease in sexual functioning during the treatment with the chemically and/or enantiomerically pure aticaprant described herein, e.g., chemically and/or enantiomerically pure crystalline Form III of aticaprant, or compositions containing the same. As used herein, the term “decrease in sexual functioning” refers to reducing or lessening of one or more components of the human sex drive, i.e., sexual functioning. In some embodiments, the sexual functioning comprises one or more of sexual drive, sexual arousal, vaginal lubrication, erection, orgasm achievement, or orgasm satisfaction. In other embodiments, the sexual functioning comprises sexual drive. In further embodiments, the sexual functioning comprises vaginal lubrication satisfaction. In further embodiments, the sexual functioning comprises orgasm achievement. In yet other embodiments, the sexual functioning comprises orgasm satisfaction. Desirably, the patient's sexual functioning is assessed at the time of initial administration of the crystalline Form III of aticaprant. Thus, the patient's sexual functioning while taking crystalline Form III of aticaprant can be compared to the patient's sexual functioning before administration of crystalline Form III of aticaprant. Sexual functioning may be assessed by using standard scales and techniques such as the Arizona Sexual Experience Scale (ASEX). The ASEX is used to investigate whether crystalline Form III of aticaprant has a further positive or negative effect on sexual function. The ASEX is 5 item rating scale administered to patients that quantifies sexual drive, sexual arousal, vaginal lubrication or penile erection, ability to reach orgasm and satisfaction. Scores range from 5 to 30, and two different versions of the scale are available (males and females).

Other scales may be utilized to determine the effectiveness of the methods used herein to treat the patient. Examples include the Cognitive and Physical Functioning Questionnaire (CPFQ), Karolinska Sleepiness Scale (KSS), and Temporal Experience of Pleasure Scale (TEPS). The CPFQ is a brief self-report scale that provides additional information regarding the impact of adjunctive treatment on aspects of cognitive and executive function including attention, memory and mental acuity. Subjects with MDD are often reported to have difficulties with functioning in this area. The KSS is a subject-reported assessment used to rate sleepiness on a scale of 1 to 9, ranging from “extremely alert” (1) to “very sleepy, great effort to keep awake, fighting sleep” (9). The TEPS includes 18 items, 2 subscales designed to distinguish between anticipatory and consummatory pleasure.

As used herein, unless otherwise noted, the term “aticaprant” refers to 3-fluoro-4-4-2-(3,5-dimethylphenyl) pyrrolidin-1-yl-methylphenoxybenzamide, i.e., the following compound:

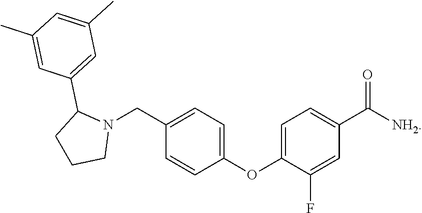 [SMILES: Cc1cc(C)cc(C2CCCN2Cc2ccc(Oc3ccc(C(N)=O)cc3F)cc2)c1]

and is also known as JNJ-67953964, CERC-501, and LY-2456302. In some embodiments, “aticaprant” refers to the(S)-enantiomer of aticaprant, i.e., the following compound:

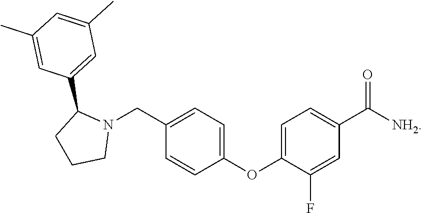 [SMILES: Cc1cc(C)cc([C@@H]2CCCN2Cc2ccc(Oc3ccc(C(N)=O)cc3F)cc2)c1]

also known as(S)-aticaprant or(S)-3-fluoro-4-4-2-(3,5-dimethylphenyl) pyrrolidin-1-yl-methylphenoxybenzamide. In other embodiments, the aticaprant used in the methods described herein is substantially free of the (R)-enantiomer, i.e., (R)-aticaprant or (R)-3-fluoro-4-4-2-(3,5-dimethylphenyl) pyrrolidin-1-yl-methylphenoxybenzamide having the following structure:

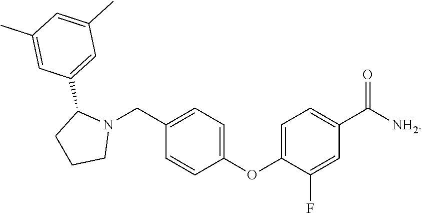 [SMILES: Cc1cc(C)cc([C@H]2CCCN2Cc2ccc(Oc3ccc(C(N)=O)cc3F)cc2)c1]

Pharmaceutically acceptable salts of aticaprant are also contemplated by the present invention, which may be readily selected by those skilled in the art. A “pharmaceutically acceptable salt” refers a salt of aticaprant that is non-toxic, biologically tolerable, or otherwise biologically suitable for administration to the subject. See, generally, G. S. Paulekuhn, “Trends in Active Pharmaceutical Ingredient Salt Selection based on Analysis of the Orange Book Database”, J. Med. Chem., 2007, 50:6665-72, S. M. Berge, “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002. Examples of pharmaceutically acceptable salts are those that are pharmacologically effective and suitable for administration to patients without undue toxicity, irritation, or allergic response.

Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, bromides (such as hydrobromides), iodides (such as hydroiodides), acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates.

In other embodiments, the chemically and/or enantiomerically pure aticaprant described herein, e.g., chemically and/or enantiomerically pure crystalline Form III of aticaprant, or compositions containing the same contains less than about 10% by weight, based on the weight of the chemically and/or enantiomerically pure aticaprant described herein, e.g., chemically and/or enantiomerically pure crystalline Form III of aticaprant, or compositions containing the same, of the (R)-enantiomer of aticaprant. In further embodiments, the chemically and/or enantiomerically pure aticaprant described herein, e.g., chemically and/or enantiomerically pure crystalline Form III of aticaprant, or compositions containing the same contains less than about 10, about 9, about 8, about 7, about 6, about 5, about 4, about 3, about 2, about 1, about 0.5, about 0.1, about 0.005, or about 0.001% by weight, based on the weight of the chemically and/or enantiomerically pure aticaprant described herein, e.g., chemically and/or enantiomerically pure crystalline Form III of aticaprant, or compositions containing the same, of the (R)-enantiomer of aticaprant. In yet other embodiments, the chemically and/or enantiomerically pure aticaprant described herein, e.g., chemically and/or enantiomerically pure crystalline Form III of aticaprant, or compositions containing the same contains about 0.001 to about 10% by weight, based on the weight of the chemically and/or enantiomerically pure aticaprant described herein, e.g., chemically and/or enantiomerically pure crystalline Form III of aticaprant, or compositions containing the same, of the (R)-enantiomer of aticaprant. In still further embodiments, the chemically and/or enantiomerically pure aticaprant described herein, e.g., chemically and/or enantiomerically pure crystalline Form III of aticaprant, or compositions containing the same contains about 0.001 to about 10%, about 0.001 to about 5%, about 0.001 to about 1%, about 0.001 to about 0.5%, about 0.001 to about 0.1%, about 0.1 to about 5%, about 0.1 to about 1%, about 0.1 to about 5%, or about 0.5 to about 5% by weight, based on the weight of the chemically and/or enantiomerically pure aticaprant described herein, e.g., chemically and/or enantiomerically pure crystalline Form III of aticaprant, or compositions containing the same.

The methods described herein include administering an effective amount of the chemically and/or enantiomerically pure aticaprant described herein, e.g., chemically and/or enantiomerically pure crystalline Form III of aticaprant. The term “effective amount” as used herein, means that amount of active compound or pharmaceutical agent that elicits the biological or medicinal response in a human that is being sought by a researcher, medical doctor or other clinician, which includes alleviation of one or more of the symptoms of the disease or disorder being treated. In some embodiments, the chemically and/or enantiomerically pure aticaprant described herein, e.g., chemically and/or enantiomerically pure crystalline Form III of aticaprant, is utilized in an effective amount as determined by the attending physician. In other embodiments, other antidepressant(s) is utilized in an effective amount either separately or in combination with the chemically and/or enantiomerically pure aticaprant described herein, e.g., chemically and/or enantiomerically pure crystalline Form III of aticaprant, or compositions containing the same.

The amount of the chemically and/or enantiomerically pure aticaprant described herein, e.g., chemically and/or enantiomerically pure crystalline Form III of aticaprant, for administration according to the methods described herein may be determined by one skill in the art and, unless otherwise noted, are set forth on a the chemically and/or enantiomerically pure aticaprant described herein, e.g., chemically and/or enantiomerically pure crystalline Form III of aticaprant, free base basis. That is, the amounts indicate that amount of the chemically and/or enantiomerically pure aticaprant described herein, e.g., chemically and/or enantiomerically pure crystalline Form III of aticaprant, molecule administered, exclusive of, for example, solvent (such as in solvates) or counterions (such as in pharmaceutically acceptable salts). In some embodiments, the effective amount of crystalline Form III of aticaprant is less than about 60 mg. In other embodiments, the effective amount of the chemically and/or enantiomerically pure aticaprant described herein, e.g., chemically and/or enantiomerically pure crystalline Form III of aticaprant, or compositions containing the same is about 0.5 mg, about 1 mg, about 2 mg, about 4 mg, about 5 mg, about 10 mg, about 15 mg, about 20 mg, 25 mg, about 30 mg, about 35 mg, about 40 mg, about 45 mg, about 50 mg, about 55 mg, or about 60 mg. In further embodiments, the effective amount of the chemically and/or enantiomerically pure aticaprant described herein, e.g., chemically and/or enantiomerically pure crystalline Form III of aticaprant, or compositions containing the same is about 1 to about 50 mg, about 5 to about 50 mg, about 10 to about 50 mg, about 20 to about 50 mg, about 30 to about 50 mg, about 40 to about 50 mg, about 1 to about 45 mg, about 2 to about 45 mg, about 5 to about 45 mg, about 10 to about 45 mg, about 20 to about 45 mg, about 30 to about 45 mg, about 30 to about 40 mg, about 30 to about 35 mg, about 1 to about 40 mg, about 5 to about 40 mg, about 10 to about 40 mg, about 20 to about 40 mg, about 30 to about 40 mg, about 1 to about 35 mg, about 2 to about 35 mg, about 5 to about 35 mg, about 10 to about 35 mg, about 20 to about 35 mg, about 25 to about 35 mg, about 30 to about 35 mg, about 1 to about 30, about 2 to about 30 mg, about 5 to about 30 mg, about 10 to about 30 mg, about 20 to about 30 mg, about 25 to about 30 mg, about 1 to about 20 mg, about 2 to about 20 mg, about 5 to about 20 mg, about 10 to about 20 mg, about 15 to about 20 mg, about 1 to about 15 mg, about 2 to about 15 mg, about 5 to about 15 mg, about 10 to about 15 mg, about 1 to about 10 mg, about 2 to about 10 mg, or about 5 to about 10 mg. In yet other embodiments, the effective amount of the chemically and/or enantiomerically pure aticaprant described herein, e.g., chemically and/or enantiomerically pure crystalline Form III of aticaprant, is about 5 to about 15 mg. In still further embodiments, the effective amount of the chemically and/or enantiomerically pure aticaprant described herein, e.g., chemically and/or enantiomerically pure crystalline Form III of aticaprant. The amount of aticaprant for administration according to the methods described herein may be determined by one skill in the art and, unless otherwise noted, are set forth on an aticaprant free base basis. That is, the amounts indicate that amount of the aticaprant molecule administered, exclusive of, for example, solvent (such as in solvates) or counterions (such as in pharmaceutically acceptable salts).

### Pharmaceutical Compositions

As used herein, the term “composition” is intended to encompass a product comprising the specified ingredients in the specified amounts, as well as any product which results, directly or indirectly, from combinations of the specified ingredients in the specified amounts. The preferred pharmaceutical composition contains the chemically and/or enantiomerically pure aticaprant described herein, e.g., crystalline Form III of aticaprant, as the active ingredient intimately admixed with a pharmaceutical carrier or excipient according to conventional pharmaceutical compounding techniques, which carrier may take a wide variety of forms depending on the form of preparation desired for administration. Suitable pharmaceutically acceptable carriers or excipients are well known in the art. Descriptions of some of these pharmaceutically acceptable carriers or excipients may be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

In certain embodiments, pharmaceutical composition for use herein, the composition further comprises one or more buffers, preservatives, penetration agents, wetting agents, surfactants, solubilizing agents, thickening agents, colorant agents, antioxidants, emulsifying agents, isotonizing agents, suspending agents, and/or viscosity increasing agents.

In some embodiments, the pharmaceutical compositions comprises one or more buffers and/or buffer systems (i.e. conjugate acid-base-pairs). As used herein, the term “buffer” shall mean any solid or liquid composition (preferably an aqueous, liquid composition) which when added to an aqueous formulation adjusts the pH of said formulation. One skilled in the art will recognize that a buffer may adjust the pH of the aqueous formulation in any direction (toward more acidic, more basic or more neutral pH). Preferably, the buffer is pharmaceutically acceptable. Suitable examples of buffers which may be used in the aqueous formulations described herein include, but are not limited to citric acid, sodium dihydrogen phosphate, disodium hydrogen phosphate, acetic acid, boric acid, sodium borate, succinic acid, tartaric acid, malic acid, lactic acid, fumaric acid, and the like.

Optionally, the pharmaceutical compositions herein may contain a preservative. As used herein, unless otherwise noted, the terms “antimicrobial preservative” and “preservative” refer to any substance that is added to pharmaceutical compositions in order to preserve them against microbial degradation or microbial growth. In this regard, microbial growth typically plays an essential role, i.e., the preservative serves the main purpose of avoiding microbial contamination. It may also be desirable to avoid any effect of the microbes on the active ingredients and excipients, respectively, i.e., to avoid microbial degradation. Representative examples of preservatives include, but are not limited to, benzalkonium chloride, benzethonium chloride, benzoic acid, sodium benzoate, benzyl alcohol, bronopol, cetrimide, cetylpyridinium chloride, chlorhexidine, chlorbutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, sodium propionate, thimerosal, methyl paraben, ethyl paraben, propyl paraben, butyl paraben, isobutyl paraben, benzyl paraben, sorbic acid, and potassium sorbate.

As used herein, the terms “penetration agent”, “penetration enhancer”, and “penetrant” refer to any substance that increases or facilitates absorption and/or bioavailability of the chemically and/or enantiomerically pure aticaprant described herein, e.g., chemically and/or enantiomerically pure crystalline Form III of aticaprant. Preferably, the penetration agent increases or facilitates absorption and/or bioavailability of the chemically and/or enantiomerically pure aticaprant described herein, e.g., chemically and/or enantiomerically pure crystalline Form III of aticaprant, following administration. Suitable examples include, but are not limited to tetradecyl maltoside, sodium glycocholate, tauroursodeoxycholic acid, lecithins, and the like; and chitosan (and salts), and surface active ingredients such as benzalkonium chloride, sodium dodecyl sulfate, sodium docusate, polysorbates, laureth-9, octoxynol, sodium deoxycholate, polyarginine, and the like. Preferably, the penetration agent is selected to meet one or more of the following general requirements:

| | |
|---|---|
| (a) | It is effective at increasing absorption of the chemically and/or |
| | enantiomerically pure aticaprant described herein, e.g., chemically |
| | and/or enantiomerically pure crystalline Form III of aticaprant, |
| | preferably in a temporary and/or reversible manner; |
| (b) | It is pharmacologically inert; |
| (c) | It is non-allergic, non-toxic and / or non-irritating; |
| (d) | It is highly potent (effective in small amounts); |
| (e) | It is compatible with the other components of the pharmaceutical |
| | composition; |
| (f) | It is odorless, colorless and / or tasteless; |
| (g) | It is accepted by regulatory agencies; and |
| (h) | It is inexpensive and available in high purity. |

The pharmaceutical compositions for use herein may further contain one or more additional excipients for example, wetting agents, surfactant components, solubilizing agents, thickening agents, colorant agents, antioxidant components, and the like.

Examples of a suitable antioxidant component, if used, include, but are not limited to one or more of the following: sulfites; ascorbic acid; ascorbates, such as sodium ascorbate, calcium ascorbate, or potassium ascorbate; ascorbyl palmitate; fumaric acid; ethylene diamine tetraacetic acid or its sodium or calcium salts; tocopherol; gallates, such as propyl gallate, octyl gallate, or dodecyl gallate; vitamin E; and mixtures thereof. The antioxidant component provides long term stability to the liquid compositions.

Solubilizing and emulsifying agents can be included to facilitate more uniform dispersion of the active ingredient or other excipient that is not generally soluble in the liquid carrier. Examples of a suitable emulsifying agent, if used, include, but are not limited to, for example, gelatin, cholesterol, acacia, tragacanth, pectin, methyl cellulose, carbomer, and mixtures thereof. Examples of suitable solubilizing agents include polyethylene glycol, glycerin, D-mannitol, trehalose, benzyl benzoate, ethanol, trisaminomethane, cholesterol, triethanolamine, sodium carbonate, sodium citrate, sodium salicylate, sodium acetate, and mixtures thereof. The solubilizing or emulsifying agent may be present in an amount sufficient to dissolve or disperse the active ingredient, i.e., the chemically and/or enantiomerically pure aticaprant described herein, e.g., chemically and/or enantiomerically pure crystalline Form III of aticaprant, in the carrier.

A suitable isotonizing agent, if used, may include sodium chloride, glycerin, D-mannitol, D-sorbitol, glucose, and mixtures thereof.

Suspending agents or viscosity increasing agents may also be added to the pharmaceutical compositions. Suitable examples include, but are not limited to, hydroxypropyl methylcellulose, sodium carmellose, microcrystalline cellulose, carbomer, pectin, sodium alginate, chitosan salts, gellan gum, poloxamer, polyvinyl pyrrolidone, xanthan gum, and the like.

Advantageously, the chemically and/or enantiomerically pure aticaprant described herein, e.g., chemically and/or enantiomerically pure crystalline Form III of aticaprant, or compositions containing the same may be administered once daily, or the total daily dosage may be administered in divided doses of two, three or four times daily.

As described herein, in particular, the patient had an inadequate response to other antidepressant therapy prior to treatment with the chemically and/or enantiomerically pure aticaprant described herein, e.g., chemically and/or enantiomerically pure crystalline Form III of aticaprant, or compositions containing the same. Thus, in a particular embodiment, the disclosure relates to the chemically and/or enantiomerically pure aticaprant described herein, e.g., chemically and/or enantiomerically pure crystalline Form III of aticaprant, or compositions containing the same, for use as described herein, wherein the patient had an inadequate response to other antidepressant therapy prior to treatment with the chemically and/or enantiomerically pure aticaprant described herein, e.g., chemically and/or enantiomerically pure crystalline Form III of aticaprant, or compositions containing the same. In a further particular embodiment, the disclosure also relates to the use of the chemically and/or enantiomerically pure aticaprant described herein, e.g., chemically and/or enantiomerically pure crystalline Form III of aticaprant, or compositions containing the same in the manufacture of a medicament, as described herein, wherein the patient had an inadequate response to other antidepressant therapy prior to treatment with the chemically and/or enantiomerically pure aticaprant described herein, e.g., chemically and/or enantiomerically pure crystalline Form III of aticaprant, or compositions containing the same. In a further particular embodiment, the disclosure further relates to a package or pharmaceutical product as described herein, wherein the patient had an inadequate response to other antidepressant therapy prior to treatment with the chemically and/or enantiomerically pure aticaprant described herein, e.g., chemically and/or enantiomerically pure crystalline Form III of aticaprant, or compositions containing the same. Such antidepressant therapy can be in particular selected from a selective serotonin reuptake inhibitor (SSRI), serotonin-norepinephrine reuptake inhibitor (SNRI), or a combination thereof.

As described herein, the chemically and/or enantiomerically pure aticaprant described herein, e.g., chemically and/or enantiomerically pure crystalline Form III of aticaprant, or compositions containing the same may be used as adjunctive treatment, or in other words, in conjunction, as an add-on, or in combination with one or more antidepressants, for example, the patient may be already, or also, administered one or more antidepressants. Thus, in a further particular embodiment, the disclosure relates to the chemically and/or enantiomerically pure aticaprant described herein, e.g., chemically and/or enantiomerically pure crystalline Form III of aticaprant, or compositions containing the same, for use as described herein, comprising administration of the chemically and/or enantiomerically pure aticaprant described herein, e.g., chemically and/or enantiomerically pure crystalline Form III of aticaprant, or compositions containing the same, as adjunctive treatment with an effective amount of one or more antidepressants. In a further particular embodiment, the disclosure relates to aticaprant, for use as described herein, comprising administration of aticaprant, in conjunction with an effective amount of one or more antidepressants. In a further particular embodiment, the disclosure relates to chemically and/or enantiomerically pure aticaprant described herein, e.g., chemically and/or enantiomerically pure crystalline Form III of aticaprant, or compositions containing the same, for use as described herein, comprising administration of the chemically and/or enantiomerically pure aticaprant described herein, e.g., chemically and/or enantiomerically pure crystalline Form III of aticaprant, or compositions containing the same, in combination with an effective amount of one or more antidepressants. In a further particular embodiment, the disclosure also relates to the use of the chemically and/or enantiomerically pure aticaprant described herein, e.g., chemically and/or enantiomerically pure crystalline Form III of aticaprant, or compositions containing the same, in the manufacture of a medicament, as described herein, wherein the treatment comprises administration of an effective amount of the chemically and/or enantiomerically pure aticaprant described herein, e.g., chemically and/or enantiomerically pure crystalline Form III of aticaprant, or compositions containing the same, as adjunctive treatment with an effective amount of one or more antidepressants. In a further particular embodiment, the disclosure also relates to the use of chemically and/or enantiomerically pure aticaprant described herein, e.g., chemically and/or enantiomerically pure crystalline Form III of aticaprant, or compositions containing the same, as described herein, wherein the treatment comprises administration of an effective amount of the chemically and/or enantiomerically pure aticaprant described herein, e.g., chemically and/or enantiomerically pure crystalline Form III of aticaprant, or compositions containing the same, in conjunction with an effective amount of one or more antidepressants. In a further particular embodiment, the disclosure also relates to the use of the chemically and/or enantiomerically pure aticaprant described herein, e.g., chemically and/or enantiomerically pure crystalline Form III of aticaprant, or compositions containing the same, as described herein, wherein the treatment comprises administration of an effective amount of the chemically and/or enantiomerically pure aticaprant described herein, e.g., chemically and/or enantiomerically pure crystalline Form III of aticaprant, in combination with an effective amount of one or more antidepressants. In a further particular embodiment, the disclosure further relates to a package or pharmaceutical product as described herein, wherein the instructions for treatment direct the administration of an effective amount of the chemically and/or enantiomerically pure aticaprant described herein, e.g., chemically and/or enantiomerically pure crystalline Form III of aticaprant, as adjunctive treatment with an effective amount of one or more antidepressants. In a further particular embodiment, the disclosure further relates to a package or pharmaceutical product as described herein, wherein the instructions for treatment direct the administration of an effective amount of the chemically and/or enantiomerically pure aticaprant described herein, e.g., chemically and/or enantiomerically pure crystalline Form III of aticaprant, in conjunction with an effective amount of one or more antidepressants. In a further particular embodiment, the disclosure further relates to a package or pharmaceutical as described herein, wherein the instructions for treatment direct administration of an effective amount of the chemically and/or enantiomerically pure aticaprant described herein, e.g., chemically and/or enantiomerically pure crystalline Form III of aticaprant, in combination with an effective amount of one or more antidepressants. Such one or more antidepressants can be selected from a selective serotonin reuptake inhibitor (SSRI), serotonin-norepinephrine reuptake inhibitor (SNRI), or a combination thereof.

As already described, the disclosure relates to the chemically and/or enantiomerically pure aticaprant described herein, e.g., chemically and/or enantiomerically pure crystalline Form III of aticaprant, or compositions containing the same, for use as described herein. In a particular embodiment, the chemically and/or enantiomerically pure aticaprant described herein, e.g., chemically and/or enantiomerically pure crystalline Form III of aticaprant, is S-aticaprant. In a further embodiment of the disclosure, the chemically and/or enantiomerically pure aticaprant described herein, e.g., chemically and/or enantiomerically pure crystalline Form III of aticaprant, in particular S-aticaprant, for use as described herein, is to be administered in an amount of about 2 to about 35 mg, more in particular, of about 10 mg. In a yet further embodiment, crystalline Form III of aticaprant, in particular S-aticaprant, for use as described herein, is administered orally. Furthermore, in a further particular embodiment, the disclosure relates to crystalline Form III of aticaprant, in particular S-aticaprant, for use as described herein, administered once daily. The disclosure also relates to the use of aticaprant, in the manufacture of a medicament, as described herein. In a particular embodiment, the chemically and/or enantiomerically pure aticaprant described herein, e.g., chemically and/or enantiomerically pure crystalline Form III of aticaprant is chemically and/or enantiomerically pure S-aticaprant. In another embodiment, the chemically and/or enantiomerically pure aticaprant described herein is chemically and/or enantiomerically pure crystalline Form III of S-aticaprant. In a further embodiment of the use as described herein, about 2 to about 35 mg the chemically and/or enantiomerically pure aticaprant described herein, e.g., chemically and/or enantiomerically pure crystalline Form III of aticaprant, is to be administered, more in particular, about 10 mg. In a yet further embodiment of the use, the chemically and/or enantiomerically pure aticaprant described herein, e.g., chemically and/or enantiomerically pure crystalline Form III of aticaprant, is to be administered orally. Furthermore, in a further particular embodiment of the use chemically and/or enantiomerically pure aticaprant described herein, e.g., chemically and/or enantiomerically pure crystalline Form III of aticaprant, or compositions containing the same in particular S-aticaprant, is to be administered once daily. In a further particular embodiment, the disclosure further relates to a package or pharmaceutical product as described herein, wherein the chemically and/or enantiomerically pure aticaprant described herein, e.g., chemically and/or enantiomerically pure crystalline Form III of aticaprant, or compositions containing the same is in particular chemically and/or enantiomerically pure S-aticaprant. In a further embodiment of the package or pharmaceutical product as described herein, the instructions for treatment direct administration of about 2 to about 35 mg the chemically and/or enantiomerically pure aticaprant described herein, e.g., chemically and/or enantiomerically pure crystalline Form III of aticaprant, more in particular, about 10 mg. In a yet further embodiment of the package or pharmaceutical product as described herein, the instructions for treatment direct the chemically and/or enantiomerically pure aticaprant described herein, e.g., chemically and/or enantiomerically pure crystalline Form III of aticaprant, in particular S-aticaprant, is for oral administration. Furthermore, in a further particular embodiment of the package or pharmaceutical product, as described herein, the instructions for treatment direct the chemically and/or enantiomerically pure aticaprant described herein, e.g., chemically and/or enantiomerically pure crystalline Form III of aticaprant, in particular S-aticaprant, is for once daily administration.

Advantageously, administration of the chemically and/or enantiomerically pure aticaprant described herein, e.g., chemically and/or enantiomerically pure crystalline Form III of aticaprant, or compositions containing the same, does not result in weight gain during treatment, including clinically relevant weight gain. Thus, in a further particular embodiment, the disclosure relates to the chemically and/or enantiomerically pure aticaprant described herein, e.g., chemically and/or enantiomerically pure crystalline Form III of aticaprant, or compositions containing the same, for use as described herein, wherein the patient does not experience weight gain during the treatment with the chemically and/or enantiomerically pure aticaprant described herein, e.g., chemically and/or enantiomerically pure crystalline Form III of aticaprant, or compositions containing the same. In a further particular embodiment, the disclosure relates to a use as defined herein, wherein the patient does not experience weight gain during the treatment with the chemically and/or enantiomerically pure aticaprant described herein, e.g., chemically and/or enantiomerically pure crystalline Form III of aticaprant, or compositions containing the same. In a further particular embodiment, the disclosure further relates to a package or pharmaceutical product as described herein, wherein the patient does not experience weight gain during the treatment with the chemically and/or enantiomerically pure aticaprant described herein, e.g., chemically and/or enantiomerically pure crystalline Form III of aticaprant, or compositions containing the same. The body weight of the patient can in particular be assessed at the time of the initial administration of aticaprant.

It was also unexpectedly observed that, based on assessment at the time of initial administration, the patient does not experience a decrease in sexual functioning during the treatment with the chemically and/or enantiomerically pure aticaprant described herein, e.g., chemically and/or enantiomerically pure crystalline Form III of aticaprant, or compositions containing the same. Thus, in further particular embodiment, the disclosure relates to the chemically and/or enantiomerically pure aticaprant described herein, e.g., chemically and/or enantiomerically pure crystalline Form III of aticaprant, or compositions containing the same, for use as described herein, wherein the patient does not experience a decrease in sexual functioning during the treatment with the chemically and/or enantiomerically pure aticaprant described herein, e.g., chemically and/or enantiomerically pure crystalline Form III of aticaprant, or compositions containing the same. In a further particular embodiment, the disclosure relates to a use as described herein, wherein the patient does not experience a decrease in sexual functioning during the treatment with the chemically and/or enantiomerically pure aticaprant described herein, e.g., chemically and/or enantiomerically pure crystalline Form III of aticaprant, or compositions containing the same. In a further particular embodiment, the disclosure relates to a package or pharmaceutical product as described herein, wherein the patient does not experience a decrease in sexual functioning during the treatment with the chemically and/or enantiomerically pure aticaprant described herein, e.g., chemically and/or enantiomerically pure crystalline Form III of aticaprant, or compositions containing the same. Such term “sexual functioning” comprises sexual drive, sexual arousal, vaginal lubrication, erection, orgasm achievement, or orgasm satisfaction. Sexual satisfaction can be assessed by methods known to the skilled person, for example, by applying the Arizona Sexual Experience Scale (ASEX).

As already described, the patient has anhedonia. In certain aspects, the anhedonia is moderate. In other aspects, the anhedonia is severe. Anhedonia can be measured, through an anhedonia scale, for example, the Snaith Hamilton Pleasure Scale (SHAPS). Thus, in a particular embodiment, the disclosure relates to the chemically and/or enantiomerically pure aticaprant described herein, e.g., chemically and/or enantiomerically pure crystalline Form III of aticaprant, or compositions containing the same, for use as described herein, wherein the anhedonia of the patient is reduced by at least 40%, as measured by the change from baseline in total score in an anhedonia scale following 6 weeks of the treatment with the chemically and/or enantiomerically pure aticaprant described herein, e.g., chemically and/or enantiomerically pure crystalline Form III of aticaprant, or compositions containing the same, more in particular, the anhedonia of the patient is reduced within about 3 weeks to about 6 weeks as measured by the change from baseline in total score in an anhedonia scale. In a further particular embodiment, the anhedonia scale is the Snaith Hamilton Pleasure Scale (SHAPS). Thus, in a particular embodiment, the disclosure relates to the use as described herein, wherein the anhedonia of the patient is reduced by at least 40%, as measured by the change from baseline in total score in an anhedonia scale following 6 weeks of the treatment with the chemically and/or enantiomerically pure aticaprant described herein, e.g., chemically and/or enantiomerically pure crystalline Form III of aticaprant, or compositions containing the same, more in particular, the anhedonia of the patient is reduced within about 3 weeks to about 6 weeks as measured by the change from baseline in total score in an anhedonia scale. In a further particular embodiment, the anhedonia scale is the Snaith Hamilton Pleasure Scale (SHAPS). In a further particular embodiment, the disclosure relates to the package or pharmaceutical product as described herein, wherein the anhedonia of the patient is reduced by at least 40%, as measured by the change from baseline in total score in an anhedonia scale following 6 weeks of the treatment with the chemically and/or enantiomerically pure aticaprant described herein, e.g., chemically and/or enantiomerically pure crystalline Form III of aticaprant, or compositions containing the same, more in particular, the anhedonia of the patient is reduced within about 3 weeks to about 6 weeks as measured by the change from baseline in total score in an anhedonia scale. In a further particular embodiment, the anhedonia scale is the Snaith Hamilton Pleasure Scale (SHAPS).

### EMBODIMENTS

The invention provides also the following non-limiting embodiments:
Embodiment 1 is a Tetrahydrofuran Solvate of Aticaprant:

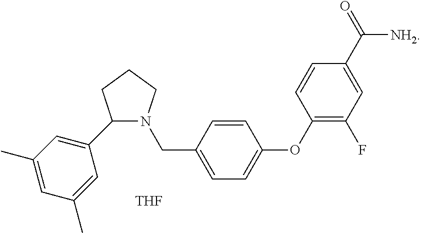 [SMILES: "THF" ; Cc1cc(C)cc(C2CCCN2Cc2ccc(Oc3ccc(C(N)=O)cc3F)cc2)c1]

Embodiment 2 is the tetrahydrofuran solvate of Embodiment 1 that is S-aticaprant:

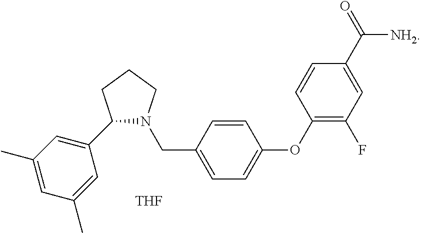 [SMILES: "THF" ; Cc1cc(C)cc([C@@H]2CCCN2Cc2ccc(Oc3ccc(C(N)=O)cc3F)cc2)c1]

Embodiment 3 is a composition comprising the tetrahydrofuran solvate of aticaprant of Embodiment 1 or 2.
Embodiment 4 is the composition of Embodiment 2, comprising about 0.10% by weight or less, based on the weight of the composition, of 3-fluoro-4-(4-formylphenoxy)benzamide:

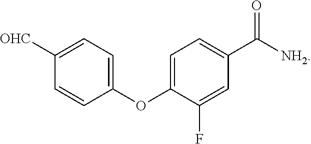 [SMILES: NC(=O)c1ccc(Oc2ccc(C=O)cc2)c(F)c1]

Embodiment 5 is the composition of Embodiment 3 or 4, comprising at least about 99.5% by weight, based on the weight of the composition, of the tetrahydrofuran solvate of aticaprant of Embodiment 1 or 2.
Embodiment 6 is the composition of any one of Embodiments 3-5, comprising less than 0.05% by weight, based on the total weight of the composition, of one or more of an organic impurity, inorganic impurity, or residual solvent.
Embodiment 7 is the composition of any one of Embodiments 3-6, comprising about 0.10% by weight or less, based on the weight of the composition, of the tetrahydrofuran solvate R-aticaprant:

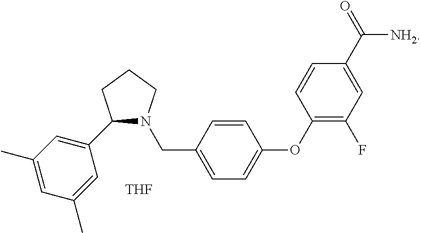 [SMILES: Cc1cc(C)cc([C@H]2CCCN2Cc2ccc(Oc3ccc(C(N)=O)cc3F)cc2)c1 ; "THF"]

Embodiment 8 is the composition of any one of Embodiments 3-7, comprising about 0.10% by weight or less, based on the weight of the composition, of R-aticaprant:

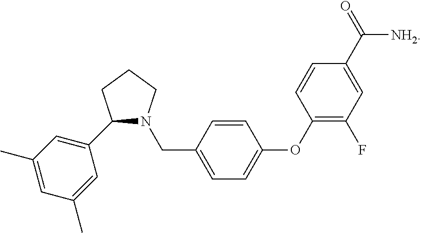 [SMILES: Cc1cc(C)cc([C@H]2CCCN2Cc2ccc(Oc3ccc(C(N)=O)cc3F)cc2)c1]

Embodiment 9 is a process for preparing the tetrahydrofuran solvate of aticaprant of Embodiment 1 or 2, comprising crystallizing aticaprant using tetrahydrofuran, ethanol, and water.
Embodiment 10 is a process for preparing the tetrahydrofuran solvate of aticaprant of Embodiment 1 or 2, comprising reacting (2S)-2-(3,5-dimethylphenyl) pyrrolidine D-tartrate

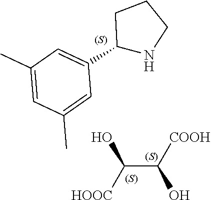

with 3-fluoro-4-(4-formylphenoxy)benzamide

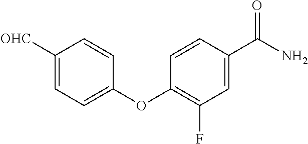 [SMILES: NC(=O)c1ccc(Oc2ccc(C=O)cc2)c(F)c1]

in the presence of tetrahydrofuran.
Embodiment 11 is a process for preparing the tetrahydrofuran solvate of aticaprant of Embodiment 1 or 2, comprising reacting (2S)-2-(3,5-dimethylphenyl) pyrrolidine D-tartrate

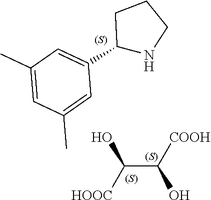

with 3-fluoro-4-(4-formylphenoxy)benzamide

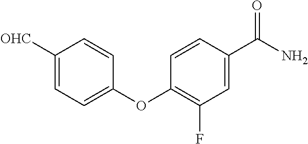 [SMILES: NC(=O)c1ccc(Oc2ccc(C=O)cc2)c(F)c1]

in the presence of a base, reducing agent, and solvent.
Embodiment 12 is the process of Embodiment 11, wherein the base is sodium hydroxide.
Embodiment 13 is the process of Embodiment 11 or 12, wherein the reducing agent is sodium triacetoxyborohydride.
Embodiment 14 is the process of any one of Embodiments 11-13, wherein the solvent is ethyl acetate, tetrahydrofuran, and 2-methyltetrahydrofuran, or a mixture thereof.
Embodiment 15 is the process of any one of Embodiments 11-14, further comprising removing the solvent and adding tetrahydrofuran.
Embodiment 16 is the process of Embodiment 15, further comprising adding ethanol and water to form a tetrahydrofuran/ethanol/water solution.
Embodiment 17 is the process of Embodiment 15 or 16, further comprising adding aticaprant seed crystals.
Embodiment 18 is the process of Embodiment 16 or 17, wherein the volume ratio of tetrahydrofuran to ethanol to water is about 1:1:2, based on the total volume of the solution.
Embodiment 19 is the process of any one of Embodiments 9-18, wherein a composition comprising the tetrahydrofuran solvate of aticaprant comprises about 0.10% by weight or less, based on the weight of the composition, of 3-fluoro-4-(4-formylphenoxy)benzamide:

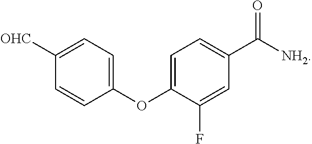 [SMILES: NC(=O)c1ccc(Oc2ccc(C=O)cc2)c(F)c1]

Embodiment 20 is a tetrahydrofuran solvate of aticaprant prepared according to the process of any one of Embodiments 9-19.
Embodiment 21 is a composition comprising a crystalline form of aticaprant and less than about 0.05% by weight, based on the weight of the composition, of 3,4-bis(4-((2-(3,5-dimethylphenyl) pyrrolidin-1-yl)methyl) phenoxy)benzamide:

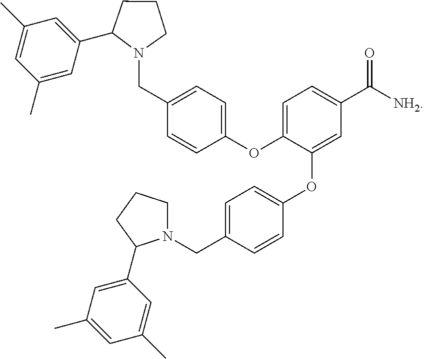 [SMILES: Cc1cc(C)cc(C2CCCN2Cc2ccc(Oc3ccc(C(N)=O)cc3Oc3ccc(CN4CCCC4c4cc(C)cc(C)c4)cc3)cc2)c1]

Embodiment 22 is the composition of Embodiment 21, wherein the crystalline form of aticaprant is characterized by four or more x-ray diffraction pattern peaks at 2θ (±0.2) of 4.1°, 9.0°, 17.6°, 18.0°, or 21.4°.
Embodiment 23 is the composition of Embodiment 21 or 22, comprising about 0.10% by weight or less, based on the weight of the composition, of 3-fluoro-4-(4-formylphenoxy)benzamide:

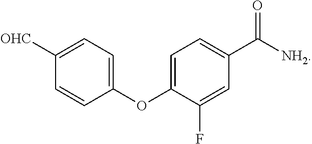 [SMILES: NC(=O)c1ccc(Oc2ccc(C=O)cc2)c(F)c1]

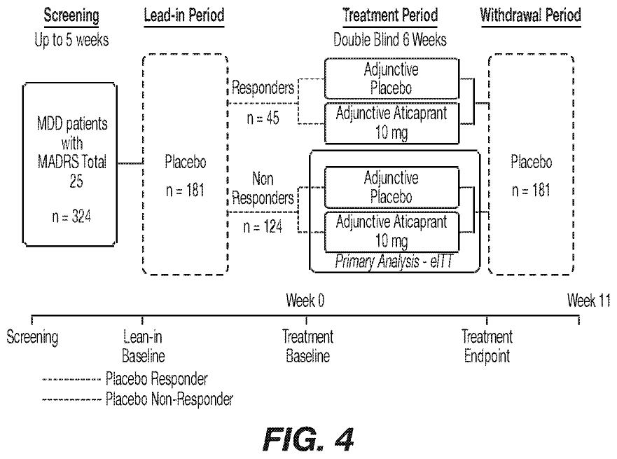

Embodiment 24 is the composition of any one of Embodiments 21-23, comprising at least about 99.5% by weight, based on the weight of the composition, of the crystalline form of aticaprant.
Embodiment 25 is the composition of any one of Embodiments 21-24, comprising about 0.10% by weight or less, based on the weight of the composition, of R-aticaprant.
Embodiment 26 is the composition of any one of Embodiments 21-25, wherein the crystalline form of aticaprant is characterized by an x-ray powder diffraction pattern that corresponds to FIG. 1.
Embodiment 27 is the composition of any one of Embodiments 21-26, wherein the crystalline form of aticaprant is characterized by a differential scanning calorimetry peak temperature (Tₘ) at about 121° C.
Embodiment 28 is the composition of any one of Embodiments 21-27, wherein the crystalline form of aticaprant is characterized by a differential scanning calorimetry thermogram that corresponds to FIG. 4.
Embodiment 29 is the composition of any one of Embodiments 21-28, wherein the crystalline form of aticaprant is anhydrous.
Embodiment 30 is a process for preparing crystalline aticaprant:

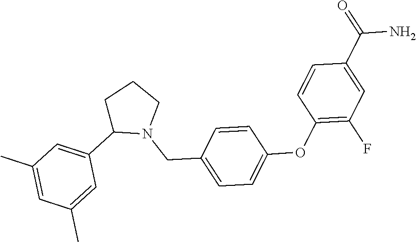 [SMILES: Cc1cc(C)cc(C2CCCN2Cc2ccc(Oc3ccc(C(N)=O)cc3F)cc2)c1]

comprising crystallizing the tetrahydrofuran solvate of aticaprant of Embodiment 1, 2, or 20 from 2-methyltetrahydrofuran and n-heptane.
Embodiment 31 is the process of Embodiment 30, wherein the crystalline aticaprant is S-aticaprant:

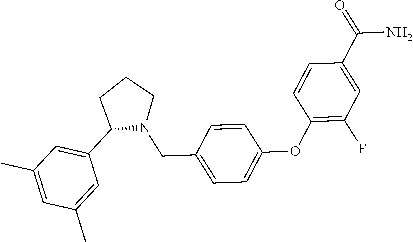 [SMILES: Cc1cc(C)cc([C@@H]2CCCN2Cc2ccc(Oc3ccc(C(N)=O)cc3F)cc2)c1]

Embodiment 32 is the process of Embodiment 30 or 31 that is performed at a temperature of less than about 48° C.
Embodiment 33 is the process of any one of Embodiments 30-32, wherein the ratio of 2-methyltetrahydrofuran to n-heptane is about 1:1 to about 1:4.
Embodiment 34 is the process of any one of Embodiments 30-33, wherein the crystalline form of aticaprant is present in a composition comprising less than about 0.05% by weight, based on the weight of the composition, of 3,4-bis(4-((2-(3,5-dimethylphenyl) pyrrolidin-1-yl)methyl) phenoxy)benzamide:

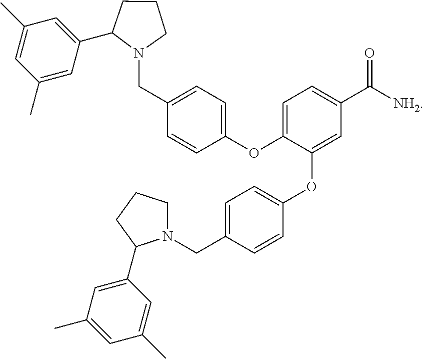 [SMILES: Cc1cc(C)cc(C2CCCN2Cc2ccc(Oc3ccc(C(N)=O)cc3Oc3ccc(CN4CCCC4c4cc(C)cc(C)c4)cc3)cc2)c1]

Embodiment 35 is the process of any one of Embodiments 30-34, wherein the crystalline form of aticaprant is characterized by four or more x-ray diffraction pattern peaks at 2θ (±0.2) of 4.1°, 9.0°, 17.6°, 18.0°, or 21.4°.
Embodiment 36 is the process of any one of Embodiments 30-35, wherein the crystalline form of aticaprant is present in a composition comprising about 0.10% by weight or less, based on the weight of the composition, of 3-fluoro-4-(4-formylphenoxy)benzamide:

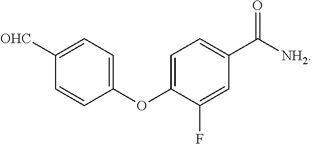 [SMILES: NC(=O)c1ccc(Oc2ccc(C=O)cc2)c(F)c1]

Embodiment 37 is the process of any one of Embodiments 30-36, comprising at least about 99.5% by weight, based on the weight of the composition, of the crystalline form of aticaprant.
Embodiment 38 is the process of any one of Embodiments 30-37, comprising about 0.10% by weight or less, based on the weight of the composition, of R-aticaprant.
Embodiment 39 is the process of any one of Embodiments 30-38, wherein the crystalline form of aticaprant is characterized by an x-ray powder diffraction pattern that corresponds to FIG. 1.
Embodiment 40 is the process of any one of Embodiments 30-39, wherein the crystalline form of aticaprant is characterized by a differential scanning calorimetry peak temperature (Tₘ) at about 121° C.
Embodiment 41 is the process of any one of Embodiments 30-40, wherein the crystalline form of aticaprant is characterized by a differential scanning calorimetry thermogram that corresponds to FIG. 4.
Embodiment 42 is the process of any one of Embodiments 30-41, wherein the crystalline form of aticaprant is anhydrous.
Embodiment 43 is a crystalline form of aticaprant prepared according to any one of Embodiments 30-42.
Embodiment 44 is a reference standard comprising the composition of any one of Embodiments 21-29 or the crystalline form of aticaprant of Embodiment 43.
Embodiment 45 is a reference standard comprising aticaprant having a purity of about 99.7%.
Embodiment 46 is the composition of any one of Embodiments 21-29, further comprising a pharmaceutically acceptable excipient.
Embodiment 47 is a method of treating major depressive disorder in human patient with the composition of any one of Embodiments 1-29 or 43 or the crystalline form of aticaprant of Embodiment 43.
Embodiment 48 is a method of treating major depressive disorder in human patient, optionally in a patient having anhedonia, comprising administering the composition of any one of Embodiments 1-29 or 43 or the crystalline form of aticaprant of Embodiment 43 to the human patient, wherein the patient had a previous inadequate response to other antidepressant therapy.
Embodiment 49 is a method of treating major depressive disorder in a human patient, comprising administering to the human patient in need thereof an effective amount of the composition of any one of Embodiments 1-29 or 43 or the crystalline form of aticaprant of Embodiment 43.
Embodiment 50 is the method of Embodiment 49, wherein the patient had an inadequate response to other antidepressant therapy prior to treatment with the composition or crystalline form of aticaprant.
Embodiment 51 is the method of any one of Embodiments 48 or 49, wherein the other antidepressant therapy is a selective serotonin reuptake inhibitor, serotonin-norepinephrine reuptake inhibitor, or a combination thereof.
Embodiment 52 is the method of any one of Embodiments 47-50, further comprising adjunctive treatment with an effective amount of one or more antidepressants.
Embodiment 53 is the method of any one of Embodiments 47-52, wherein the one or more antidepressants is a selective serotonin reuptake inhibitor, serotonin-norepinephrine reuptake inhibitor, or a combination thereof.
Embodiment 54 is the method of any one of Embodiments 47-53, wherein the effective amount of the crystalline form of aticaprant is about 2 to about 35 mg.
Embodiment 55 is the method of Embodiment 54, wherein the effective amount of the crystalline form of aticaprant is about 10 mg.
Embodiment 56 is the method of any one of Embodiments 47-55, wherein the composition or crystalline form of aticaprant is administered orally.
Embodiment 57 is the method of any one of Embodiments 47-56, wherein the composition or crystalline form of aticaprant is administered once daily.
Embodiment 58 is the method of any one of Embodiments 47-57, wherein the patient has anhedonia.
Embodiment 59 is the method of any one of Embodiments 47-58, wherein the patient has moderate anhedonia.
Embodiment 60 is the method of any one of Embodiments 47-58, wherein the patient has severe anhedonia.
Embodiment 61 is the method of any one of Embodiments 47-60, wherein the patient does not experience weight gain during the treatment with the composition or crystalline form of aticaprant.
Embodiment 62 is the method of Embodiment 61, wherein patient's body weight is assessed at the time of the initial administration of the composition or crystalline form of aticaprant.
Embodiment 63 is the method of any one of Embodiments 47-62, wherein the patient does not experience a decrease in sexual functioning during the treatment with the crystalline form of aticaprant.
Embodiment 64 is the method of Embodiment 63, wherein the sexual functioning of the patient is assessed at the time of initial administration of the composition or crystalline form of aticaprant.
Embodiment 65 is the method of Embodiment 63 or 64, wherein the sexual functioning comprises sexual drive, sexual arousal, vaginal lubrication, erection, orgasm achievement, or orgasm satisfaction.
Embodiment 66 is the method of any one of Embodiments 63-65, wherein sexual functioning is assessed by the Arizona Sexual Experience Scale.
Embodiment 67 is the method of any one of Embodiments 59-61, wherein the anhedonia of the patient is reduced by at least 40%, as measured by the change from baseline in total score in an anhedonia scale following 6 weeks of the treatment with the composition or crystalline form of aticaprant.
Embodiment 68 is the method of any one of Embodiments 59-61 and 67, wherein the anhedonia of the patient is reduced within about 3 weeks to about 6 weeks as measured by the change from baseline in total score in an anhedonia scale.
Embodiment 69 is the method of Embodiment 67 or 68, wherein the anhedonia scale is the Snaith Hamilton Pleasure Scale.
Embodiment 70 is the composition of any one of Embodiments 1-29 or 43 or the crystalline form of aticaprant of Embodiment 43 for use in treating major depressive disorder in a human patient, optionally in a patient having anhedonia.
Embodiment 71 is the composition of any one of Embodiments 1-29 or 43 or the crystalline form of aticaprant of Embodiment 43 for use treating major depressive disorder in human patient, wherein the patient had a previous inadequate response to other antidepressant therapy.
Embodiment 72 is the composition of any one of Embodiments 1-29 or 43 or the crystalline form of aticaprant of Embodiment 43 for use in treating major depressive disorder in a human patient.
Embodiment 73 is the composition or crystalline form of aticaprant of Embodiment 72, wherein the patient had an inadequate response to other antidepressant therapy prior to treatment with the composition or crystalline form of aticaprant.
Embodiment 74 is the composition or crystalline form of aticaprant of any one of Embodiments 71 or 72, wherein the other antidepressant therapy is a selective serotonin reuptake inhibitor, serotonin-norepinephrine reuptake inhibitor, or a combination thereof.
Embodiment 75 is the composition or crystalline form of aticaprant of any one of Embodiments 70-74, further comprising adjunctive treatment with an effective amount of one or more antidepressants.
Embodiment 76 is the composition or crystalline form of aticaprant of any one of Embodiments 70-75, wherein the one or more antidepressants is a selective serotonin reuptake inhibitor, serotonin-norepinephrine reuptake inhibitor, or a combination thereof.
Embodiment 77 is the composition or crystalline form of aticaprant of any one of Embodiments 70-76, wherein the effective amount of the crystalline form of aticaprant is about 2 to about 35 mg.
Embodiment 78 is the composition or crystalline form of aticaprant of Embodiment 77, wherein the effective amount of the crystalline form of aticaprant is about 10 mg.
Embodiment 79 is the composition or crystalline form of aticaprant of any one of Embodiments 70-78, wherein the composition or crystalline form of aticaprant is administered orally.
Embodiment 80 is the composition or crystalline form of aticaprant of any one of Embodiments 70-79, wherein the composition or crystalline form of aticaprant is administered once daily.
Embodiment 81 is the composition or crystalline form of aticaprant of any one of Embodiments 70-80, wherein the patient has anhedonia.
Embodiment 82 is the composition or crystalline form of aticaprant of any one of Embodiments 70-81, wherein the patient has moderate anhedonia.
Embodiment 83 is the composition or crystalline form of aticaprant of any one of Embodiments 70-81, wherein the patient has severe anhedonia.
Embodiment 84 is the composition or crystalline form of aticaprant of any one of Embodiments 70-83, wherein the patient does not experience weight gain during the treatment with the composition or crystalline form of aticaprant.
Embodiment 85 is the composition or crystalline form of aticaprant of Embodiment 84, wherein patient's body weight is assessed at the time of the initial administration of the composition or crystalline form of aticaprant.
Embodiment 86 is the composition or crystalline form of aticaprant of any one of Embodiments 70-85, wherein the patient does not experience a decrease in sexual functioning during the treatment with the crystalline form of aticaprant.
Embodiment 87 is the composition or crystalline form of aticaprant of Embodiment 86, wherein the sexual functioning of the patient is assessed at the time of initial administration of the composition or crystalline form of aticaprant.
Embodiment 88 is the composition or crystalline form of aticaprant of Embodiment 86 or 87, wherein the sexual functioning comprises sexual drive, sexual arousal, vaginal lubrication, erection, orgasm achievement, or orgasm satisfaction.
Embodiment 89 is the composition or crystalline form of aticaprant of any one of Embodiments 86-88, wherein sexual functioning is assessed by the Arizona Sexual Experience Scale.
Embodiment 90 is the composition or crystalline form of aticaprant of any one of Embodiments 81-83, wherein the anhedonia of the patient is reduced by at least 40%, as measured by the change from baseline in total score in an anhedonia scale following 6 weeks of the treatment with the composition or crystalline form of aticaprant.
Embodiment 91 is the composition or crystalline form of aticaprant of any one of Embodiments 81-83 and 90, wherein the anhedonia of the patient is reduced within about 3 weeks to about 6 weeks as measured by the change from baseline in total score in an anhedonia scale.
Embodiment 92 is the composition or crystalline form of aticaprant of Embodiment 90 or 91, wherein the anhedonia scale is the Snaith Hamilton Pleasure Scale.
Embodiment 93 is the use of the composition of any one of Embodiments 1-29 or 43 or the crystalline form of aticaprant of Embodiment 43 for treating major depressive disorder in a human patient having anhedonia.
Embodiment 94 is the use of any one of Embodiments 1-29 or 43 or the crystalline form of aticaprant of Embodiment 43 for treating major depressive disorder in human patient, wherein the patient had a previous inadequate response to other antidepressant therapy.
Embodiment 95 is the use of the composition of any one of Embodiments 1-29 or 43 or the crystalline form of aticaprant of Embodiment 43 for treating major depressive disorder in a human patient.
Embodiment 96 is the use of Embodiment 95, wherein the patient had an inadequate response to other antidepressant therapy prior to treatment with the composition or crystalline form of aticaprant.
Embodiment 97 is the use of any one of Embodiments 94 or 95, wherein the other antidepressant therapy is a selective serotonin reuptake inhibitor, serotonin-norepinephrine reuptake inhibitor, or a combination thereof.
Embodiment 98 is the use of any one of Embodiments 93-97, further comprising adjunctive treatment with an effective amount of one or more antidepressants.
Embodiment 99 is the use any one of Embodiments 93-98, wherein the one or more antidepressants is a selective serotonin reuptake inhibitor, serotonin-norepinephrine reuptake inhibitor, or a combination thereof.
Embodiment 100 is the use of any one of Embodiments 93-98, wherein the effective amount of the crystalline form of aticaprant is about 2 to about 35 mg.
Embodiment 101 is the use of Embodiment 100, wherein the effective amount of the crystalline form of aticaprant is about 10 mg.
Embodiment 102 is the use of any one of Embodiments 93-101, wherein the composition or crystalline form of aticaprant is administered orally.
Embodiment 103 is the use of any one of Embodiments 93-102, wherein the composition or crystalline form of aticaprant is administered once daily.
Embodiment 104 is the use of any one of Embodiments 93-103, wherein the patient has anhedonia.
Embodiment 105 is the use of any one of Embodiments 93-104, wherein the patient has moderate anhedonia.
Embodiment 106 is the use of any one of Embodiments 93-105, wherein the patient has severe anhedonia.
Embodiment 107 is the use of any one of Embodiments 93-106, wherein the patient does not experience weight gain during the treatment with the composition or crystalline form of aticaprant.
Embodiment 108 is the use of Embodiment 107, wherein patient's body weight is assessed at the time of the initial administration of the composition or crystalline form of aticaprant.
Embodiment 109 is the use of any one of Embodiments 93-108, wherein the patient does not experience a decrease in sexual functioning during the treatment with the crystalline form of aticaprant.
Embodiment 110 is the use of Embodiment 109, wherein the sexual functioning of the patient is assessed at the time of initial administration of the composition or crystalline form of aticaprant.
Embodiment 111 is the use of Embodiment 109 or 110, wherein the sexual functioning comprises sexual drive, sexual arousal, vaginal lubrication, erection, orgasm achievement, or orgasm satisfaction.
Embodiment 112 is the use of any one of Embodiments 100-111, wherein sexual functioning is assessed by the Arizona Sexual Experience Scale.
Embodiment 113 is the use of any one of Embodiments 104-106, wherein the anhedonia of the patient is reduced by at least 40%, as measured by the change from baseline in total score in an anhedonia scale following 6 weeks of the treatment with the composition or crystalline form of aticaprant.
Embodiment 114 is the use of any one of Embodiments 104-106 and 113, wherein the anhedonia of the patient is reduced within about 3 weeks to about 6 weeks as measured by the change from baseline in total score in an anhedonia scale.
Embodiment 115 is the use of Embodiment 113 or 114, wherein the anhedonia scale is the Snaith Hamilton Pleasure Scale.
Embodiment 116 is a package or pharmaceutical product comprising (i) composition of any one of Embodiments 1-29 or 43 or the crystalline form of aticaprant of Embodiment 43, and (ii) instructions for treating major depressive disorder in a human patient having anhedonia.

The following Examples are set forth to aid in the understanding of the invention, and are not intended and should not be construed to limit in any way the invention set forth in the claims which follow thereafter.

Abbreviations

| Acronym | Meaning | Acronym | Meaning |
|---|---|---|---|
| RH | relative humidity | RT | room temperature |
| DSC | differential scanning | mDSC | modulated differential |
| | calorimetry | | scanning calorimetry |
| THF | tetrahydrofuran | XRPD | x-ray powder diffraction |

### Example 1: Instrument and Methodology Details

### A. X-Ray Powder Diffraction (XRPD)

### Bruker AXS D8 Advance

XRPD diffractograms were collected on a Bruker D8 diffractometer using Cu Kα radiation (40 kV, 40 mA) and a 0-20 goniometer fitted with a Ge monochromator. The incident beam passes through a 2.0 mm divergence slit followed by a 0.2 mm anti-scatter slit and knife edge. The diffracted beam passes through an 8.0 mm receiving slit with 2.5° Soller slits followed by the Lynxeye Detector. The software used for data collection and analysis was Diffrac Plus XRD Commander and Diffrac Plus EVA respectively.

Samples were run under ambient conditions as flat plate specimens using powder as received. The sample was prepared on a polished, zero-background (510) silicon wafer by gently pressing onto the flat surface or packed into a cut cavity. The sample was rotated in its own plane.

The details of the standard Pharmorphix data collection method are:
Angular range: 2 to 42° 2θ
Step size: 0.05° 2θ
Collection time: 0.5 s/step (total collection time: 6.40 min)

When required other methods for data collection are used with details as shown in Table 7.

TABLE 7
Additional D8 XRPD methods
4 Minute Method

| | |
|---|---|
| Angular Range | 2 to 31° 2θ |
| Step Size | 0.06° 2θ |
| Time per Step | 0.5 s/step |

### PANalytical Empyrean

XRPD diffractograms were collected on a PANalytical Empyrean diffractometer using Cu Kα radiation (45 kV, 40 mA) in transmission geometry. A 0.5° slit, 4 mm mask and 0.04 rad Soller slits with a focusing mirror were used on the incident beam. A PIXcel^{3D} detector, placed on the diffracted beam, was fitted with a receiving slit and 0.04 rad Soller slits. The software used for data collection was X'Pert Data Collector using X'Pert Operator Interface. The data were analyzed and presented using Diffrac Plus EVA or HighScore Plus.

Samples were prepared and analyzed in either a metal or Millipore 96 well-plate in transmission mode. X-ray transparent film was used between the metal sheets on the metal well-plate and powders (approximately 1-2 mg) were used as received. The Millipore plate was used to isolate and analyze solids from suspensions by adding a small amount of suspension directly to the plate before filtration under a light vacuum.

The scan mode for the metal plate used the gonio scan axis, whereas a 20 scan was utilized for the Millipore plate.

The details of the standard screening data collection method are:
Angular range: 2.5 to 32.0° 2θ
Step size: 0.0130° 2θ
Collection time: 12.75 s/step (total collection time of 2.07 min)

The software used for data collection was X'Pert Data Collector and the data analyzed and presented using Diffrac Plus EVA.

### B. Differential Scanning Calorimetry (DSC)

### Ta Instruments Q2000

DSC data were collected on a TA Instruments Q2000 equipped with a 50 position auto-sampler. Typically, 0.5-3 mg of each sample, in a pin-holed aluminum pan, was heated at 10° C./min from 25° C. to 275° C. A purge of dry nitrogen at 50 mL/min was maintained over the sample.

Modulated temperature DSC was carried out using an underlying heating rate of 2° C./min and temperature modulation parameters of +0.636° C. (amplitude) every 60 seconds.

The instrument control software was Advantage for Q Series and Thermal Advantage and the data were analyzed using Universal Analysis or TRIOS.

### TA Instruments Discovery DSC

DSC data were collected on a TA Instruments Discovery DSC equipped with a 50 position auto-sampler. Typically, 0.5-3 mg of each sample, in a pin-holed aluminum pan, was heated at 10° C./min from 25° C. to 275° C. A purge of dry nitrogen at 50 mL/min was maintained over the sample.

The instrument control software was TRIOS and the data were analyzed using TRIOS or Universal Analysis.

### C. Chemical Purity Determination by HPLC

Purity analysis was performed on an Agilent HP1100/Infinity II 1260 series system equipped with a diode array detector and using OpenLAB software. The full method details are provided in Table 8.

TABLE 8
HPLC method for chemical purity determinations

| Parameter | Value | | |
|---|---|---|---|
| Type of method | Reverse phase with gradient elution | | |
| Sample Preparation | 0.2 mg/ml in acetonitrile:water 1:1 | | |
| Column | Supelco Ascentis Express C18 2.7 μm | | |
| | 100 × 4.6 mm | | |
| Column Temperature (° C.) | 25 | | |
| Injection (μL) | 5 | | |
| Detection: Wavelength, | 255, 90 | | |
| Bandwidth (nm) | | | |
| Flow Rate (ml/min) | 2 | | |
| Phase A | 0.1% TFA in water | | |
| Phase B | 0.085% TFA in acetonitrile | | |
| | Time (min) | % Phase A | % Phase B |
| Timetable | 0 | 95 | 5 |
| | 6 | 5 | 95 |
| | 6.2 | 95 | 5 |
| | 8 | 95 | 5 |

### Example 2—Analytical Procedures

A. Identification and Quantitative Determination of the Aticaprant and Purities by HPLC Methods. See, Table 10.

TABLE 10

| | |
|---|---|
| Method A | Method B |

| | | |
|---|---|---|
| Column | Zorbax SB-C18, | Chiralpak AD-H, |
| | 150 × 4.6 mm, 5 μm | 150 × 4.6 mm, 5 μm mm |
| | or equivalent | or equivalent |

| | | | | |
|---|---|---|---|---|
| Column | 30° | C. | 30° | C. |
| Temperature | | | | |
| Flow Rate | 1.0 | mL/min | 1.0 | mL/min |
| Injection | 20 | μL | 10 | μL |
| volume | | | | |

| | | |
|---|---|---|
| Detection | UV at 250 nm | UV at 250 nm |
| Mobile | A: 0.1% TFA in Water | n-Hexane:Isopropyl |
| Phase | B: 0.1% TFA in | Alcohol:Diethylamine |
| | Acetonitrile | |
| | | Isocratic |
| Elution | Gradient | |
| Mode | | |

### B. Determination of Residual Solvents by Gas Chromatography (GC). See, Table 11 for the Operating Conditions and Table 12 for the Validation Summary.

TABLE 11
GC Operating Conditions

| | n-heptane, ethyl | |
|---|---|---|
| | acetate, and ethanol | 2-methyl-tetrahydrofuran |
| Column | DB-624, 75 m × | 6% cyanopropylphenyl/94% |
| | 3.0.53 mm × 0 μm | methylpolysiloxane 20 m × |
| | or equivalent | 0.18 mm, 1.0 μm film |
| | | thickness or equivalent |
| Carrier Gas | Helium | N₂ or equivalent |
| Flow Rate | 5 mL/min | 1.0 mL/min constant flow |
| Injector | Split | Headspace |
| Detector | FID | FID |
| Autosampler | Liquid injection | N/A |

TABLE 12
Method Validation Summary

| | n-heptane, ethyl acetate, | |
|---|---|---|
| | and ethanol | 2-methyl-tetrahydrofuran |
| Specificity | Separation between the | Separation between the residual |
| | Residual Solvents. | solvents is demonstrated. The results |
| | | prove the specificity of the method. |
| Linearity | 500-6000 ppm: r² ≥ 0.99 | 54-10864 ppm range: correlation |
| | | coefficient r ≥ 0.99 RSD response |
| | | factors ≤15.0%. |
| Precision | System Repeatability RSD ≤10% | System repeatability: ≤10.0% |
| | (n = 6) Analysis Repeatability | Analysis repeatability at 10xRT- |
| | RSD ≤10% (n = 6) | level: ≤15.0% |
| Accuracy | Recovery between 70.0-130% at | Recovery at RT-level between 50.0- |
| | Reporting Threshold (RT), | 150.0% Recovery at 10xRT-level |
| | Reference, and 1.2 x Reference | between 70.0-130.0% Recovery at |
| | level | 100xRT-level between 80.0-120.0% |
| | | Recovery at 200xRT-level between |
| | | 80.0-120.0%. |
| Range | 500-6000 ppm | 54-10864 ppm. |
| LOQ | 500 ppm (RT) | QL = Reporting Threshold |
| | | Mean recovery between 50.0-150.0% |
| | | % RSD: ≤25.0% |
| LOD | ≤⅓ LOQ | DL = 27 ppm |
| Stability | Reporting solutions were shown | Solution stability is demonstrated for |
| | to be stable for 7 days at ambient | the sample solution during a period of |
| | conditions Standard solutions | 1 day in the autosampler. |
| | were shown to be stable for 3 | Solution stability is demonstrated |
| | days at ambient conditions | for the reference solution during a |
| | Sample solutions were shown to | period of 2 months, if stored at |
| | be stable for 5 days at ambient | ambient temperature and protected |
| | conditions. | from daylight. |

### Example 3—Preparation of the Aticaprant THF Solvate

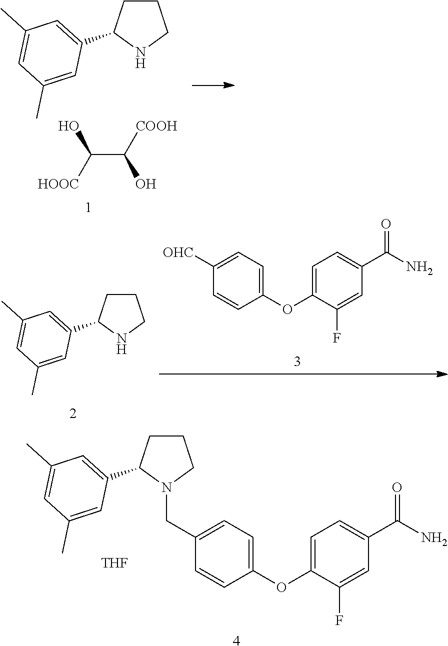

Aqueous sodium hydroxide was added to compound 1 in 2-methyltetrahydrofuran. After phase separation, compound 2, i.e., the free base of compound 1, in 2-MeTHF was solvent switched to tetrahydrofuran (THF). Reductive amination of compound 3 using compound 2 was carried out by adding sodium triacetoxyborohydride and THF. Upon reaction completion, the reaction mixture was washed with saturated sodium bicarbonate and sodium chloride. The organic phase containing crude compound 4 was concentrated and ethanol and water were added. The product was crystallized using THF, ethanol, and water to produce compound 4 as a solid.

To a reactor vessel at room temperature with stirring was added water (7.3 L/kg), by 2-methyltetrahydrofuran (6.6 L/kg), and (2S)-2-(3,5-dimethylphenyl) pyrrolidine D-tartrate (1.15 mole/mole), followed by NaOH (2.3 mole/mole 50% aq.) over a minimum of 20 minutes at 22° C. The reactor was rinse with water (1.0 L/kg) and the mixture was stirred for a minimum of 30 minutes at 22° C., then it was allowed to settle for a minimum of 30 minutes. The layers were separated and the lower aqueous layer was discarded. The organic layer was concentrated under vacuum to minimal volume at a maximum of 45° C. THF was charged portion-wise and distilled back to minimal volume to complete the solvent switch. The temperature was then adjusted to 20° C. and THF (5.0 L/kg) was added, followed by 3-fluoro-4-(4-formylphenoxy)benzamide (1.00 mol) and additional THF (10.0 L/kg). The temperature was adjusted to 32° C. and the mixture was stirred for a minimum of 1 h. The temperature was then adjusted to 15° C., then NaBH(OAc)₃ (1.50 mole/mole) was added portion-wise at 15° C. The mixture was further stirred for a minimum of 1 h at 15° C. Then water (5.0 L/kg) was added at 15° C., followed by NaOH (3.05 mole/mole 50% aq.) at 15° C. and the resulting mixture was further stirred for a minimum of 2 h. The reactor was rinsed with water (0.5 L/kg) and the mixture was allowed to settle for a minimum of 30 min. The lawyers were separated and the lower aqueous layer was discarded. NaCl (6.2 L/kg 20% aq.) was then added to the organic layer at 15° C. and stirred for minimum 30 min, then the mixture was allowed to settle for minimum 30 minutes, the layers were separated and the lower aqueous layer was discarded. The organic layer was concentrated under vacuum to 8.0 L/kg at maximum 45° C. The temperature was adjusted to 25° C., then EtOH (2% MeOH) (8.0 L/kg) and water (8.0 L/kg) were added at 25° C. Additional water (2.0 L/kg) was added at 25° C. over minimum 2.5 h. Crude aticaprant seeds (0.03 kg/kg) were added at 25° C. and the mixture was stirred for minimum 2 h at 25° C. Additional water (6.0 L/kg) was added at 25° C. over minimum 7.5 h. Then the mixture was cooled to 2.5° C. over minimum 7 h and stirred for a further minimum 6 h at 2.5° C. The product was isolated, washed with THF/EtOH/water 1:1:2 (volume ratio) and dried at 25° C.

### Example 4—Preparation of Pure Aticaprant

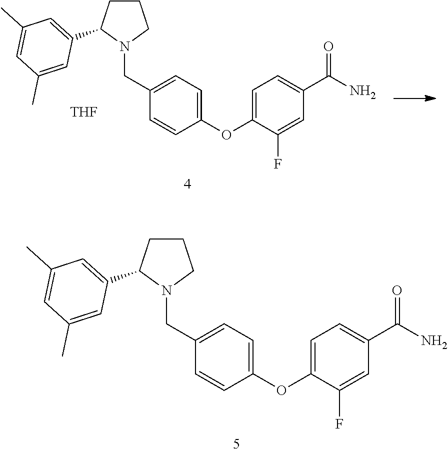 [SMILES: Cc1cc(C)cc([C@@H]2CCCN2Cc2ccc(Oc3ccc(C(N)=O)cc3F)cc2)c1 ; "THF" ; "4" ; Cc1cc(C)cc([C@@H]2CCCN2Cc2ccc(Oc3ccc(C(N)=O)cc3F)cc2)c1 ; "5"]

To a stirred solution of 2-methyltetrahydrofuran and n-heptane in a reactor vessel was added crude aticaprant (1.0 mol) using the 2-methyl-tetrahydrofuran and n-heptane ratios noted in Table 13.

TABLE 13

| Temperature | Solvent ratio | Crystallization | Theoretical |
|---|---|---|---|
| (° C.) | (w/w) | Volume | yield (w/w %) |
| 40 | 5/95 | 113.5 | 96 |
| 30 | 1/3 | 22 | 84 |
| | 1/4 | 27.4 | 90 |
| | 1/5 | 33.2 | 93 |
| | 1/7 | 44.7 | 95 |
| 25 | 1/3 | 22 | 86 |
| | 1/4 | 27.4 | 92 |
| 20 | 1/3 | 22 | 89 |
| 10 | 35/65 | 15.2 | 82 |
| | 30/70 | 17.9 | 87 |

Stirring was continued, the temperature was adjusted to 42° C. and the mixture was stirred for minimum 10 minutes until full dissolution. The solution was filtered over a polish filter into a crystallization reactor to remove insoluble matter that might be present and the polish filter was rinsed. The solution was stirred for minimum 10 min at 40° C. then cooled as noted in Table 13 over a minimum 1 h, then stirred for minimum 10 minutes at the same temperature. n-Heptane (was added over minimum 30 minutes. The mixture was stirred for minimum 10 minutes, then seeded with aticaprant (0.02 mole/mole) and further stirred for minimum 8 h. Additional n-heptane was added over a minimum of 12 h and the mixture was stirred for minimum 2 h, then cooled to 10° C. over minimum 3 h and stirred for minimum 4 hours at 10° C. The product was then isolated, the resulting wet cake was washed with 2-methyltetrahydrofuran/n-heptane (25/75 w/w %), and dried at 50° C. Characterization of the product showed MS m/z 419/2 and elemental analysis as shown in Table 14.

TABLE 14
Elemental Analysis

| | C (%) | H (%) | N (%) |
|---|---|---|---|
| Theoretical | 74.62 | 6.50 | 6.69 |
| Measured | 74.45 | 6.68 | 6.63 |
| Absolute Difference | 0.17 | 0.18 | 0.06 |

HPLC of the samples from Examples 3 and 4 was performed as shown in Table 15.

TABLE 15

| Instrument | Agilent 1260 | | |
|---|---|---|---|
| Software | Agilent OpenLAB v2.0 | | |
| Column | Zorbax SB-C18, 150 × 4.6 mm, | | |
| | 5 μm or equivalent | | |
| Wavelength | 250 nm | | |
| Flow rate | 1.0 mL/min | | |
| Column Temperature | 30° C. | | |
| Injection volume | 20 μL | | |
| Analysis time | 35 min | | |
| Mobile phase | A: 0.01 V % TFA aqueous | | |
| | B: 0.01 V % TFA CH₃CN | | |
| Gradient | Time | A | B |
| | Initial | 90% | 10% |
| | 25 min | 30% | 70% |
| | 28 min | 30% | 70% |
| | 28.1 min | 90% | 10% |
| | 35 min | 90% | 10% |

The results in Table 16 show that the claimed process provide highly chemical pure samples of aticaprant.

TABLE 16
HPLC purity results

| | |
|---|---|
| Description | 15.2 |
| Example 3 aticaprant | 99.31 |
| Example 4 aticaprant | 99.82 |

Characterization was performed on compound 5 and the results are shown in Table 17.

TABLE 17
Characterization data for Form III

| | |
|---|---|
| XRPD | Crystalline |
| HPLC Purity | 99.3% |
| DSC | Endo. 121.0° C., 75 J/g. |
| | Degradation not observed |
| TGA | No weight loss observed before degradation. |
| | Degradation onset ~250° C. |
| GVS | 0.4% max uptake at 90% RH |
| | No hysteresis observed |
| | XRPD unchanged after GVS |

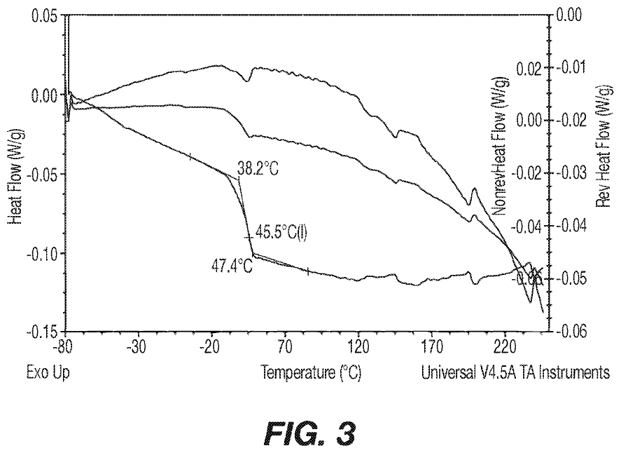

Form III of aticaprant was found to be crystalline by XRPD. 1H NMR showed that the material was consistent with the proposed structure, with the presence of residual ethyl acetate. Ion chromatography showed that there were no cations/anions present, and HPLC showed 99.8% purity. The DSC (heating from 20 to 131° C. at 10° C./min) showed a peak temperature at 121° C. See, FIG. 3.

The levels of R-aticaprant, impurities, residual solvents, residues, and water were determined. See, Table 18.

TABLE 18
Batch Analyses

| Test | Acceptance | Batches | | | | |
|---|---|---|---|---|---|---|
| Parameter | Criteria | 1 | 2 | 3 | 4 | 5 |
| Assay (% w/w) | 97.0-103.0% on | 98.4 | 100.1 | 100.1 | 100.1 | 100.0 |
| | dry basis | | | | | |
| (R)-Enantiomer | NMT 1.5% | 0.2 | <0.05 | <0.05 | <0.05 | <0.05 |
| Chiral Impurity | | | | | | |
| (% area) | | | | | | |

Chromatographic Purity (% w/w)^{b}

| | | | | | | |
|---|---|---|---|---|---|---|
| Impurity 1 | NMT 0.20% | 0.20 | 0.05 | <0.05 | <0.05 | <0.05 |
| Unspecified | NMT 0.10% | RRT 1.11- | 0.05 | RRT | RRT | RRT |
| Impurities | | 1.13: 0.16 | | 0.92: 0.05 | 1.41: 0.09 | 1.41: 0.09 |
| | | RRT 1.25- | | | | |
| | | 1.31: 0.08 | | | | |
| Total Impurities | NMT 2.0% | 0.44 | 0.19 | 0.05 | 0.09 | 0.09 |

Residual Solvents (%)

| | | | | | | |
|---|---|---|---|---|---|---|
| n-Heptane | NMT 5000 ppm | NT | <503 | <503 | 824 | 740 |
| Ethyl acetate | NMT 5000 ppm | NT | 1072 | 1167 | <505 | <505 |
| Ethanol | NMT 5000 ppm | 0.03^{c} | <501 | ND | <501 | <501 |
| 2-Methyl- | NMT 5000 ppm | NT | <54 | <54 | 1145 | 1063 |
| tetrahydrofuran | | | | | | |
| Residue on | NMT 0.3% | NT | 0.0 | 0.0 | 0.0 | 0.0 |
| Ignition | | | | | | |
| Water | NMT 1.0% w/w | NT | <0.1 | <0.1 | <0.1 | <0.1 |

TABLE 18
Batch Analyses

| Test | Acceptance | Batches | | | | | |
|---|---|---|---|---|---|---|---|
| Parameter | Criteria | 6 | 7 | 8 | 9 | 10 | 11 |
| Assay (% w/w) | 97.0-103.0% on | 100.1 | 100.1 | 100.2 | 100.0 | 100.2 | 100.0 |
| | dry basis | | | | | | |
| (R)-Enantiomer | NMT 1.5% | <0.05 | <0.05 | <0.05 | <0.05 | <0.05 | <0.05 |
| Chiral Impurity | | | | | | | |
| (% area) | | | | | | | |

Chromatographic Purity (% w/w)^{b}

| | | | | | | | |
|---|---|---|---|---|---|---|---|
| Impurity 1 | NMT 0.20% | <0.05 | <0.05 | <0.05 | <0.05 | <0.05 | <0.05 |
| Unspecified | NMT 0.10% | RRT | RRT | RRT | <0.05 | <0.05 | RRT |
| Impurities | | 1.41: 0.09 | 1.41: 0.09 | 1.20: 0.06 | | | 1.41: 0.10 |
| Total Impurities | NMT 2.0% | 0.09 | 0.09 | 0.06 | <0.05 | <0.05 | 0.10 |

Residual Solvents (%)

| | | | | | | | |
|---|---|---|---|---|---|---|---|
| n-Heptane | NMT 5000 ppm | 700 | 825 | <503 | <503 | <503 | 538 |
| Ethyl acetate | NMT 5000 ppm | <505 | <505 | <505 | <505 | <505 | <505 |
| Ethanol | NMT 5000 ppm | <501 | <501 | <501 | <501 | <501 | <501 |
| 2-Methyl- | NMT 5000 ppm | 1069 | 1147 | 674 | 643 | 642 | 940 |
| tetrahydrofuran | | | | | | | |
| Residue on 19 | NMT 0.3% | 0.0 | 0.0 | 0.0 | 0.0 | 0.0 | 0.0 |
| Ignition | | | | | | | |
| Water | NMT 1.0% w/w | <0.1 | <0.1 | 0.0 | <0.1 | <0.1 | <0.1 |

The impurities of the drug substance were identified. There are 5 impurities (Impurity 1, RRT 1.12, RRT 1.20, RRT 1.41, and RRT 0.92), apart from the toxicological batches, at or above the reporting threshold of 0.05%. Impurity 1 was the only impurity observed above the identification threshold of 0.10%, which was manufactured according to SM 1.1.

The structure for impurity 1 is Table 19 and was determined by high resolution mass spectrometry (HRMS) and NMR.

TABLE 19

| Impurity | | | |
|---|---|---|---|
| RRTª | Impurity | Impurity Structure | Class^{b} |
| 1.15-1.18 | Impurity 1 | 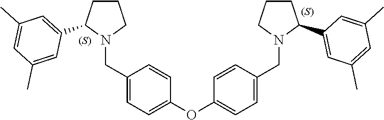 | Class 5 |

^{a}RRT = Relative Retention Time;
^{b}Refer to ICH M7 Guideline: Assessment and Control of DNA Reactive (Mutagenic) Impurities in Pharmaceuticals to Limit Potential Carcinogenic Risk., Adopted: 25 Sep. 2014; Published in the Federal Register, 28 May 2015, Vol. 80, No 102, p. 30465.

In summary, the levels of inorganic impurities, i.e., residue on ignition/sulphated ash, are consistently below or equal to 0.3%. Further, the levels of the residual solvents are consistently below the ICH Q3C limits and the levels of water are consistently below the specification limit of NMT 1.0%.

### Example 5

This was a multi-center, placebo-controlled, randomized, double-blind study in subjects with MDD who have had an inadequate response to SSRI/SNRI treatment. Aticaprant was evaluated as an adjunctive therapy; therefore, eligible subjects were maintained on their SSRI/SNRI treatment without change throughout the study. At least 50% of recruited subjects had to be anhedonic (as measured by SHAPS total score ≥20).

### A. Objectives

The primary objective was to evaluate the efficacy of aticaprant compared to placebo when administered as adjunctive treatment in subjects with MDD partially responsive to SSRI/SNRI treatment in terms of reduction of symptoms of depression, as assessed by the change from baseline on the MADRS in non-responders during the placebo lead-in period.

### The Secondary Objectives are:

i. To evaluate the efficacy of aticaprant compared to placebo when administered as adjunctive treatment in subjects with MDD partially responsive to SSRI/SNRI treatment in terms of reduction of symptoms of depression, as assessed by the change from baseline on the MADRS in both responders and non-responders during the placebo lead-in period.
ii. To investigate the overall safety and tolerability of treatment with adjunctive aticaprant in subjects with MDD when used in combination with a SSRI or SNRI.
iii. To investigate the effect of aticaprant versus placebo on depression related anhedonia as assessed by the SHAPS.
iv. To investigate the effect of aticaprant on symptoms of depression using the Clinical Global Impression-Severity (CGI-S), the patient reported Symptoms of Major Depressive Disorder Scale (SMDDS) and the self-assessment of treatment experience (SATE).
v. To investigate the effect of aticaprant on symptoms of anxiety using the HAM-A and on core symptoms of anxiety using the HAM-A6 subscale.
vi. To assess the plasma PK of aticaprant in subjects with MDD and explore its relationship with efficacy and safety parameters.

### Secondary Exploratory Objectives Include:

i. To explore the effect of aticaprant on aspects of cognitive and executive function using the CPFQ.
ii. To explore mood-related biomarkers (including but not limited to growth factors, HPA axis markers, immune system activation, metabolic markers) and genetic/epigenetic variation that may be related to clinical response, nonresponse, or safety and tolerability parameters of aticaprant.

### B. Study Design

For each subject, the study consisted of two phases: a screening phase of up to 5 weeks and a double-blind treatment phase lasting 11 weeks. See, FIG. 4.

Subjects with MDD who have had treatment initiated with a permitted SSRI/SNRI and have had an inadequate or only partial response to this treatment were screened. Assessments include the MINI, Antidepressant Treatment History Questionnaire (TRQ), and MADRS.

The treatment phase consisted of 3 periods. A placebo lead-in period of concealed duration, after which subjects entered the double-blind treatment period when they were randomly assigned to 10 mg aticaprant (two 5 mg capsules) or continue placebo for 6 weeks. Each capsule contained aticaprant (5 mg), microcrystalline cellulose (94.95 mg), and magnesium stearate (0.05 mg) in a hard gelatin capsule. Subjects who completed the treatment period, entered the withdrawal period and were treated with placebo for the remaining time of the treatment phase. The total duration for each subject was approximately 16 weeks. There were 11 scheduled visits, including screening. An overall flow diagram is shown in FIG. 4.

Subjects were screened within 35 to 2 days prior to Day 1 to ascertain their eligibility per the inclusion and exclusion criteria. The symptoms of depression were assessed using the structured interview guide for the MADRS.

### Double-Blind Treatment Phase

The duration of the double-blind treatment phase was 11 weeks divided into 3 periods. The subject received medication after completion of the visit on Day 1. The first dose was taken at home on Day 2. All medication was taken in fasting condition. At Visits 3, 4 and 5, the subjects were re-randomized to blind subjects the duration of the placebo lead-in period. During the double-blind phase, the subjects visited the center for out-patient visits every 1 to 2 weeks. See, Table 20.

TABLE 20
Time and Events Schedule (TES)

| | Phase | | | | | | | | | | |
|---|---|---|---|---|---|---|---|---|---|---|---|
| | Screening | ^{a}Double-blind treatment phase | | | | | | | | | |
| | Visit number | | | | | | | | | | |
| | | | | | | | | | | | ^{b}11 |
| | 1 | 2 | 3 | 4 | 5 | 6 | 7 | 8 | 9 | 10 | or EW |
| | Week (end of) | | | | | | | | | | |
| | −5 to 0 | 0 | 1 | 2 | 3 | 4 | 6 | 7 | 8 | 9 | 11 |
| | Day | | | | | | | | | | |
| | −3 5 to −2 | 1 | 8 | 15 | 22 | 29 | 43 | 50 | 57 | 64 | 78 |
| Safety assessments | | | | | | | | | | | |
| Physical and | X | X | | | | X | | | | | X |
| neurological | | | | | | | | | | | |
| examination | | | | | | | | | | | |
| ASEX | | X | | | X | X | X | | | X | X |
| KSS | | X | | | X | X | X | | | X | X |
| Suicidality by C-SSRS | X | X | X | X | X | X | X | X | X | X | X |
| Dosing | | | | | | | | | | | |
| Randomization | | X | X | X | X | | | | | | |
| Supply new medication | | X | X | X | X | X | X | X | X | X | |
| Oral dose medication^{d} | | Day 2 until and including Day 78^{e} | | | | | | | | | |
| Meal after dosing | | X^{l} | X^{l} | X^{l} | X^{l} | X^{l} | X^{l} | X^{l} | X^{l} | X^{l} | X^{l} |
| Clinical Assessments | | | | | | | | | | | |
| Structured Interview | X^{j} | X | X | X | X | X | X | X | X | X | X |
| Guide MADRS | | | | | | | | | | | |
| Structured Interview | | X | X | X | X | X | X | X | X | X | X |
| Guide SIGH-A | | | | | | | | | | | |
| CGI-S | | X | X | X | X | X | X | X | X | X | X |
| SMDDS | | X | | | X | X | X | | | X | |
| CPFQ | | X | | | X | X | X | | | X | |
| SHAPS | X | X | X | X | X | X | X | X | X | X | X |
| SATE^{k} | | once weekly while at home | | | | | | | | | |
| Ongoing subject review | | | | | | | | | | | |
| Assessment of subject | X | up to 3 occasion when at home | | | | | | | | | |
| engagement^{k} | | | | | | | | | | | |

| | |
|---|---|
| Adverse events | Continuous |
| Concomitant medication | Continuous |

EW = early withdrawal;
^{a}Visits should be conducted ±3 days of the scheduled day (based on Visit 2, not based on previous visit).
^{b}If a subject discontinues treatment before the end of the double-blind treatment phase, EW visit should be completed.
^{d}At home: In fasting condition. At clinic visit days: Use blisters dispensed at the previous visit. In fasting condition after completion of predose assessments.
^{e}When Visit 11 is planned up to 3 days later, continue medication.
^{j}During the first screening visit and by telephone up to 4 days before Visit 2, if 2 weeks or more elapse between the MADRS rating at screening and Visit 2.
^{k}Using Q1.6-app on subjects' smartphone.
^{l}Breakfast, lunch or dinner after drug intake at site.

Lead-in period: Subjects who successfully complete the baseline examination visit at the clinical site/unit, were treated with placebo for the entire duration of the lead-in period.

Treatment period: At the end of the lead-in period both placebo lead-in responders and placebo lead-in non-responders were randomized to receive either placebo or 10 mg aticaprant in a 1:1 ratio for 6 weeks. Subjects remained blinded to exact timing of the randomization, response criterion and drug treatment assignment for each subject.

Withdrawal period: Subjects who completed the double-blind treatment period prior to the end of Week 11 entered the withdrawal period where they were treated with placebo for the remaining time of the treatment phase.

### C. Dosage and Administration

Aticaprant was supplied as 5-mg capsules. Placebo was supplied as matching capsules. All subjects took 2 capsules QD. The capsules were taken daily from Day 2 to Day 78 in fasting condition with some water (fasting for at least 4 hours before dosing). Medication was taken before breakfast. If the subject has forgotten to take the medication before breakfast, this was done before the next following meal, at the latest at dinner of the same day. If the subject remembered later than dinner, the dose of that day was omitted, and the subject took the dose before breakfast on the next day.

When Visit 11 was planned up to 3 days later, the subject continued medication until Visit 11.

The capsules were swallowed whole and not chewed, divided, dissolved or crushed. After having taken the medication, subjects did not to eat or drink for at least 30 minutes.

The first dose was taken in fasting condition on Day 2 of the double-blind phase. The dose of the medication was:
10 mg aticaprant: 2 capsules of 5 mg aticaprant
Placebo: 2 placebo capsules.

Medication dose was adjusted as needed to 5 mg QD based on the results of a blinded review of the safety data. When a dose reduction has been decided on, this only applied to new subjects and the dose of medication was:
5 mg aticaprant: 1 capsule of 5 mg aticaprant
Placebo: 1 placebo capsule.

As used herein, the Enriched ITT Analysis Set (eITT) is defined as all enrolled lead-in placebo non-responders who were randomized into a treatment period, received at least one dose of study medication in the treatment period and have at least one post-baseline MADRS assessment during the treatment period. Similarly, the Full ITT Analysis Set (fITT) is defined as all enrolled subjects who were randomized into a treatment period, received at least one dose of study medication in the treatment period and have at least one post-treatment baseline assessment of MADRS during the treatment period.

### D. Clinical Assessments

(i) Depression: Montgomery-Åsberg Depression Rating Scale (MADRS), Clinical Global Impression-Severity (CGI-S), Symptoms of Major Depressive Disorder Scale (SMDDS), and Self-assessment of treatment experience (SATE)
(ii) Anhedonia: Snaith-Hamilton Pleasure Scale (SHAPS)
(iii) Anxiety: Structured Interview Guide for the Hamilton Anxiety scale (SIGH-A) and HAM-A6
(iv) Effects on Cognition: The Cognitive and Physical Functioning Questionnaire (CPFQ)
(v) Safety assessments

Standard safety assessments including physical and neurological examination, vital signs, 12-lead ECG, clinical chemistry, hematology, and urinalysis was performed. Based on observations of GI complaints in previous studies, a panel including PGI, PGII, G17 and Hp IgG was added to the clinical laboratory test panel to test for stomach mucosa status.
(vi) Suicidal ideation: C-SSRS
(vii) Exploratory: CPFQ
(viii) Central sedating effects: Karolinska Sleepiness Scale
(ix) Sexual dysfunction: ASEX

### E. Patient Population

Of 184 subjects, 169 were randomized into the treatment period and included in the safety population, while 166 subjects were considered for the full ITT population. Out of the 166 subjects in the full ITT population, 121 (73%) were lead-in placebo non-responders (enriched ITT population) and the remaining 45 (27%) were lead-in placebo responders. Of the 121 subjects in the enriched population, 112 (92.6%) were white and 84 (69.4%) were female. The mean age was 41.6 years, ranging from 19 to 64 years. All subjects had anhedonia (defined as SHAPS total score ≥20) at treatment baseline. A high anhedonia level (defined as SHAPS total score ≥38) was observed in 43.8% of the subjects. In general, the treatment groups were similar with respect to the baseline characteristics. Subject demographics for the eITT and safety analysis are provided in Tables 21 and 22.

TABLE 21
Summary of Demographics and Baseline
Characteristics; Full Safety Analysis

| | aticaprant | |
|---|---|---|
| Placebo | 10 mg | Total |
| (N = 84) | (N = 85) | (N = 169) |

Age (Years)

| N | 84 | | 85 | | 169 | |
|---|---|---|---|---|---|---|
| Mean (SD) | 42.1 | (12.54) | 43.0 | (12.81) | 42.6 | (12.65) |

| Median | 43.5 | 43.0 | 43.0 |
|---|---|---|---|
| Range | (19; 64) | (21; 64) | (19; 64) |

Gender

| N | 84 | | 85 | | 169 | |
|---|---|---|---|---|---|---|
| Female | 62 | (73.8%) | 60 | (70.6%) | 122 | (72.2%) |
| Male | 22 | (26.2%) | 25 | (29.4%) | 47 | (27.8%) |

Race

| N | 84 | | 85 | | 169 | |
|---|---|---|---|---|---|---|
| American Indian | 1 | (1.2%) | 0 | | 1 | (0.6%) |
| or Alaska Native | | | | | | |
| Asian | 2 | (2.4%) | 2 | (2.4%) | 4 | (2.4%) |
| Black or African | 2 | (2.4%) | 5 | (5.9%) | 7 | (4.1%) |
| American | | | | | | |
| White | 79 | (94.0%) | 78 | (91.8%) | 157 | (92.9%) |

Ethnicity

| N | 84 | | 85 | | 169 | |
|---|---|---|---|---|---|---|
| Hispanic or | 10 | (11.9%) | 13 | (15.3%) | 23 | (13.6%) |
| Latino | | | | | | |
| Not Hispanic | 74 | (88.1%) | 72 | (84.7%) | 146 | (86.4%) |
| or Latino | | | | | | |

Country

| N | 84 | | 85 | | 169 | |
|---|---|---|---|---|---|---|
| Germany | 4 | (4.8%) | 5 | (5.9%) | 9 | (5.3%) |
| Moldova | 15 | (17.9%) | 14 | (16.5%) | 29 | (17.2%) |
| Russia | 25 | (29.8%) | 21 | (24.7%) | 46 | (27.2%) |
| Ukraine | 9 | (10.7%) | 7 | (8.2%) | 16 | (9.5%) |
| United Kingdom | 10 | (11.9%) | 15 | (17.6%) | 25 | (14.8%) |
| United States | 21 | (25.0%) | 23 | (27.1%) | 44 | (26.0%) |

Baseline Height (cm)

| N | 84 | | 85 | | 169 | |
|---|---|---|---|---|---|---|
| Mean (SD) | 167.4 | (7.91) | 168.2 | (8.64) | 167.8 | (8.27) |

| Median | 167.5 | 167.6 | 167.6 |
|---|---|---|---|
| Range | (150; 183) | (152; 195) | (150; 195) |

Baseline Weight (kg)

| N | 84 | | 85 | | 169 | |
|---|---|---|---|---|---|---|
| Mean (SD) | 76.2 | (14.73) | 78.7 | (15.23) | 77.4 | (14.99) |

| Median | 75.3 | 78.9 | 77.1 |
|---|---|---|---|
| Range | (47; 116) | (42; 119) | (42; 119) |

Baseline BMI (kg/m²)

| N | 84 | | 85 | | 169 | |
|---|---|---|---|---|---|---|
| Mean (SD) | 27.2 | (4.92) | 27.7 | (4.56) | 27.5 | (4.73) |

| Median | 26.6 | 28.1 | 27.6 |
|---|---|---|---|
| Range | (19; 35) | (18; 35) | (18; 35) |

Presence of Anhedonia at Baseline

| N | 84 | | 85 | | 169 | |
|---|---|---|---|---|---|---|
| No | 0 | | 1 | (1.2%) | 1 | (0.6%) |
| Yes | 84 | (100.0%) | 84 | (98.8%) | 168 | (99.4%) |

Lead-in response status

| N | 84 | | 85 | | 169 | |
|---|---|---|---|---|---|---|
| No | 62 | (73.8%) | 62 | (72.9%) | 124 | (73.4%) |
| Yes | 22 | (26.2%) | 23 | (27.1%) | 45 | (26.6%) |

TABLE 22
Summary of Demographics and Baseline Characteristics; eITT

| | aticaprant | |
|---|---|---|
| Placebo | 10 mg | Total |
| (N = 61) | (N = 60) | (N = 121) |

Age (Years)

| N | 61 | | 60 | | 121 | |
|---|---|---|---|---|---|---|
| Mean (SD) | 41.6 | (12.34) | 41.6 | (12.78) | 41.6 | (12.51) |

| Median | 43.0 | 40.5 | 42.0 |
|---|---|---|---|
| Range | (19; 64) | (21; 64) | (19; 64) |

Gender

| N | 61 | | 60 | | 121 | |
|---|---|---|---|---|---|---|
| Female | 42 | (68.9%) | 42 | (70.0%) | 84 | (69.4%) |
| Male | 19 | (31.1%) | 18 | (30.0%) | 37 | (30.6%) |

Race

| N | 61 | | 60 | | 121 | |
|---|---|---|---|---|---|---|
| American | 1 | (1.6%) | 0 | | 1 | (0.8%) |
| Indian or | | | | | | |
| Alaska Native | | | | | | |
| Asian | 2 | (3.3%) | 1 | (1.7%) | 3 | (2.5%) |
| Black or | 2 | (3.3%) | 3 | (5.0%) | 5 | (4.1%) |
| African | | | | | | |
| American | | | | | | |
| White | 56 | (91.8%) | 56 | (93.3%) | 112 | (92.6%) |

Ethnicity

| N | 61 | | 60 | | 121 | |
|---|---|---|---|---|---|---|
| Hispanic or | 3 | (4.9%) | 7 | (11.7%) | 10 | (8.3%) |
| Latino | | | | | | |
| Not Hispanic | 58 | (95.1%) | 53 | (88.3%) | 111 | (91.7%) |
| or Latino | | | | | | |

Country

| N | 61 | | 60 | | 121 | |
|---|---|---|---|---|---|---|
| Germany | 4 | (6.6%) | 4 | (6.7%) | 8 | (6.6%) |
| Moldova | 15 | (24.6%) | 14 | (23.3%) | 29 | (24.0%) |
| Russia | 19 | (31.1%) | 18 | (30.0%) | 37 | (30.6%) |
| Ukraine | 7 | (11.5%) | 5 | (8.3%) | 12 | (9.9%) |
| United | 6 | (9.8%) | 10 | (16.7%) | 16 | (13.2%) |
| Kingdom | | | | | | |
| United States | 10 | (16.4%) | 9 | (15.0%) | 19 | (15.7%) |

Baseline Height (cm)

| N | 61 | | 60 | | 121 | |
|---|---|---|---|---|---|---|
| Mean (SD) | 168.1 | (8.19) | 167.3 | (8.10) | 167.7 | (8.13) |

| Median | 168.0 | 166.3 | 167.0 |
|---|---|---|---|
| Range | (151; 183) | (152; 186) | (151; 186) |

Baseline Weight (kg)

| N | 61 | | 60 | | 121 | |
|---|---|---|---|---|---|---|
| Mean (SD) | 74.7 | (14.19) | 76.8 | (15.12) | 75.7 | (14.63) |

| Median | 74.2 | 77.1 | 75.6 |
|---|---|---|---|
| Range | (47; 116) | (42; 119) | (42; 119) |

Baseline BMI (kg/m²)

| N | 61 | | 60 | | 121 | |
|---|---|---|---|---|---|---|
| Mean (SD) | 26.4 | (4.67) | 27.3 | (4.36) | 26.9 | (4.52) |

| Median | 25.7 | 27.8 | 26.7 |
|---|---|---|---|
| Range | (19; 35) | (18; 35) | (18; 35) |

Presence of Anhedonia at Baseline

| N | 61 | | 60 | | 121 | |
|---|---|---|---|---|---|---|
| No | 0 | | 0 | | 0 | |
| Yes | 61 | (100.0%) | 60 | (100.0%) | 121 | (100.0%) |

Lead-in response status

| N | 61 | | 60 | | 121 | |
|---|---|---|---|---|---|---|
| No | 61 | (100.0%) | 60 | (100.0%) | 121 | (100.0%) |

| | | | |
|---|---|---|---|
| Yes | 0 | 0 | 0 |

### E. Evaluations of Efficacy

At the end of the lead-in period, response status of the subjects was assessed according to the double-blind response criteria based on reduction in MADRS relative to lead-in baseline. Both lead-in placebo responders and lead-in placebo non-responders were randomly assigned in a 1:1 ratio to either aticaprant or placebo in the treatment period. The randomization was stratified by lead-in response status (non-responders: <30% reduction from baseline in MADRS total score at the end of the lead-in period vs responders: ≥30% reduction from baseline at the end of the lead-in period) and presence/absence of anhedonia (presence defined as SHAPS total score ≥20).

Treatment duration: The study consisted of two periods: a screening phase of up to 5 weeks and a double-blind treatment phase of 11 weeks. The double-blind treatment phase of the trial consisted of 3 periods. The first period was a placebo lead-in of 3 weeks, after which subjects entered the treatment period when they were randomly assigned to aticaprant or continuation on placebo for 6 weeks. Subjects who successfully completed the treatment period were treated with placebo during a 2-week withdrawal period, i.e., Period 3. The total duration for each subject was approximately 16 weeks.

Primary analysis set for efficacy: The efficacy analysis is based on the eITT set defined as all enrolled lead-in placebo non-responders who were randomized into the treatment period, received at least one dose of medication, and have at least one post-baseline MADRS assessment during the treatment period. The primary analysis set is used for all efficacy endpoints.

Secondary analysis set for efficacy: A secondary analysis set is the fITT set defined as all enrolled subjects who were randomized into the treatment period, received at least one dose of medication, and have at least one post-baseline MADRS assessment during the treatment period. The secondary analysis set is used for all efficacy endpoints to examine the effect in the general population, which may be useful for designing subsequent studies in the development program.

Analysis set for safety: The safety analysis is based on the full safety analysis set, defined as all enrolled subjects who received at least one dose of medication in the treatment period.

The efficacy endpoints were presented for both the eITT and the fITT.

Level of significance: The analysis of primary efficacy endpoint was performed at a significance level of 0.20 (one-sided). The analysis of secondary efficacy endpoints was performed at a significance level of 0.20 (two-sided). No adjustment for multiple comparisons was performed.

### F. Results

(i) Primary Endpoint: Change from Treatment Baseline in MADRS Total Score at Treatment Week 6 in Non-Responders during Placebo Lead-in Period

### Enriched ITT Analysis Set

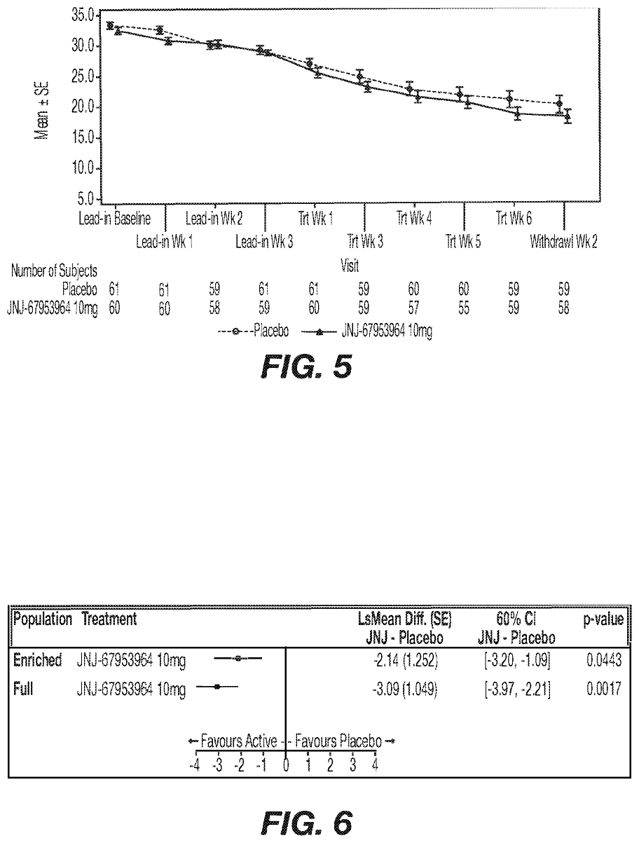

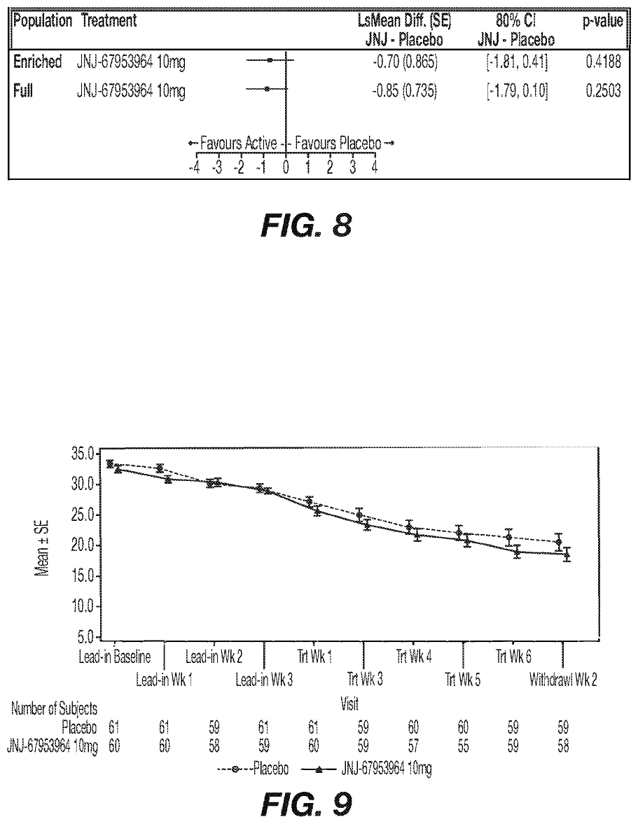

The mean (SD) MADRS total score at treatment baseline was 29.0 (4.61), ranging from 19 to 41. See, FIG. 5. The mean change from treatment baseline (SD) in MADRS total score at treatment week 6 was −10.2 (8.44) for aticaprant and −8.2 (8.53) for placebo. The observed effect size was 0.23. See, Tables 23-25 and FIG. 8.

TABLE 23
Summary of Baseline Psychiatry Rating Scales at the Start
of the Lead-in and Treatment Periods; eITT Analysis Set

| MADRS | | | SHAPS | | |
|---|---|---|---|---|---|
| Total Score | | | Total Score | | |
| | Mean | Median | | Mean | Median |
| N | (SD) | (Range) | N | (SD) | (Range) |

Lead-in Baseline

| | | | | | | |
|---|---|---|---|---|---|---|
| Placebo | 61 | 33.4 (4.25) | 34.0 (26; 42) | 61 | 38.0 (6.28) | 38.0 (22; 55) |
| Aticaprant | 60 | 32.5 (4.18) | 32.0 (25; 45) | 60 | 38.3 (5.66) | 38.0 (21; 53) |
| Total | 121 | 32.9 (4.22) | 33.0 (25; 45) | 121 | 38.1 (5.96) | 38.0 (21; 55) |

Treatment Baseline

| | | | | | | |
|---|---|---|---|---|---|---|
| Placebo | 61 | 29.2 (5.47) | 29.0 (19; 41) | 61 | 36.8 (5.75) | 37.0 (23; 50) |
| Aticaprant | 60 | 28.7 (3.58) | 28.5 (21; 36) | 60 | 36.4 (5.16) | 36.5 (20; 49) |
| Total | 121 | 29.0 (4.61) | 29.0 (19; 41) | 121 | 36.6 (5.45) | 37.0 (20; 50) |

TABLE 24
MADRS Total Score: Mean Changes to Placebo During
the Treatment Period; eITT Analysis Set

| | | | | | |
|---|---|---|---|---|---|
| | | Mean | Mean | 90% CI | |
| Analysis | | Change from | Change to | for Mean | |
| Visit | | Baseline | Placebo | Change to | Effect |
| Treatment | N | (SD) | (SD pooled) | Placebo | Size |

Treatment Week 1

| | | | | | |
|---|---|---|---|---|---|
| Placebo | 61 | −2.2 (3.73) | | | |
| Aticaprant | 60 | −3.3 (5.21) | −1.1 (4.52) | [−2.4, 0.3] | −0.24 |

Treatment Week 3

| | | | | | |
|---|---|---|---|---|---|
| Placebo | 59 | −4.3 (5.99) | | | |
| Aticaprant | 59 | −5.7 (6.38) | −1.4 (6.18) | [−3.3, 0.5] | −0.22 |

Treatment Week 4

| | | | | | |
|---|---|---|---|---|---|
| Placebo | 60 | −6.4 (6.66) | | | |
| Aticaprant | 57 | −7.3 (7.35) | −0.9 (7.00) | [−3.1, 1.2] | −0.14 |

Treatment Week 5

| | | | | | |
|---|---|---|---|---|---|
| Placebo | 60 | −7.4 (7.15) | | | |
| Aticaprant | 55 | −8.4 (7.36) | −1.1 (7.25) | [−3.3, 1.2] | −0.14 |

Treatment Week 6

| | | | | | |
|---|---|---|---|---|---|
| Placebo | 59 | −8.2 (8.53) | | | |
| Aticaprant | 59 | −10.2 (8.44) | −2.0 (8.49) | [−4.6, 0.6] | −0.23 |

Negative change from baseline indicates improvement. Negative change to Placebo indicates favorable aticaprant effect. Negative effect size favors aticaprant; positive effect size favors Placebo.

TABLE 25
MADRS Total Score: MMRM Results - Estimated LS Means
and Comparison versus Placebo; eITT Analysis Set

| | | | | | | | |
|---|---|---|---|---|---|---|---|
| | | | | | LSMean | 60% | |
| Analysis | | | | | Difference | Confidence | |
| Visit | | Mean | Mean | LSMean | (SE)\Treatment | Interval on | p- |
| Treatment | N | (SD) | (SD) | (SE) | Placebo | Difference | value^{a} |

Treatment Week 1

| | | | | | | | |
|---|---|---|---|---|---|---|---|
| Placebo | 61 | 26.9 | −2.2 | −2.0 | | | |
| | | (6.77) | (3.73) | (0.92) | | | |
| aticaprant | 60 | 25.4 | −3.3 | −3.2 | −1.2 | [−2.28, | 0.1604 |
| | | (5.93) | (5.21) | (0.93) | (1.24) | −0.19] | |

Treatment Week 3

| | | | | | | | |
|---|---|---|---|---|---|---|---|
| Placebo | 59 | 24.8 | −4.3 | −4.2 | | | |
| | | (8.25) | (5.99) | (0.92) | | | |
| aticaprant | 59 | 23.1 | −5.7 | −5.6 | −1.5 | [−2.55, | 0.1159 |
| | | (6.58) | (6.38) | (0.93) | (1.25) | −0.44] | |

Treatment Week 4

| | | | | | | | |
|---|---|---|---|---|---|---|---|
| Placebo | 60 | 22.7 | −6.4 | −6.2 | | | |
| | | (9.10) | (6.66) | (0.92) | | | |
| aticaprant | 57 | 21.5 | −7.3 | −7.3 | −1.1 | [−2.19, | 0.1811 |
| | | (7.49) | (7.35) | (0.93) | (1.25) | −0.09] | |

Treatment Week 5

| | | | | | | | |
|---|---|---|---|---|---|---|---|
| Placebo | 60 | 21.7 | −7.4 | −7.2 | | | |
| | | (9.54) | (7.15) | (0.92) | | | |
| aticaprant | 55 | 20.5 | −8.4 | −8.7 | −1.5 | [−2.60, | 0.1103 |
| | | (7.44) | (7.36) | (0.94) | (1.25) | −0.48] | |

Treatment Week 6

| | | | | | | | |
|---|---|---|---|---|---|---|---|
| Placebo | 59 | 20.9 | −8.2 | −8.0 | | | |
| | | (10.54) | (8.53) | (0.92) | | | |
| aticaprant | 59 | 18.6 | −10.2 | −10.1 | −2.1 | [−3.20, | 0.0443 |
| | | (8.14) | (8.44) | (0.93) | (1.25) | −1.09] | |

^{a}One-sided test for no difference between treatments from a MMRM model with subject as random effect; country, treatment, time and time-by-treatment interaction as factors; and baseline MADRS total score as continuous covariate. An AR(1) variance-covariance matrix was employed

Based on the results of a MMRM model with subject as random effect; country, treatment, time and time-by-treatment interaction as factors; and baseline MADRS total score as continuous covariate a significant positive efficacy signal was detected for aticaprant versus placebo at the one-sided 0.20 significance level. The estimated LS mean difference at treatment week 6 between aticaprant and placebo was −2.1 with 80% 1-sided CI upper limit of −1.09. The corresponding p-value was 0.044. The treatment effect was larger in the fITT than in the eITT population: −3.1 with 80% 1-sided CI upper limit of −2.2 (p=0.002). The effect size was 0.36 and 0.23, respectively. See, FIGS. 2 and 3.

### Full ITT Analysis Set

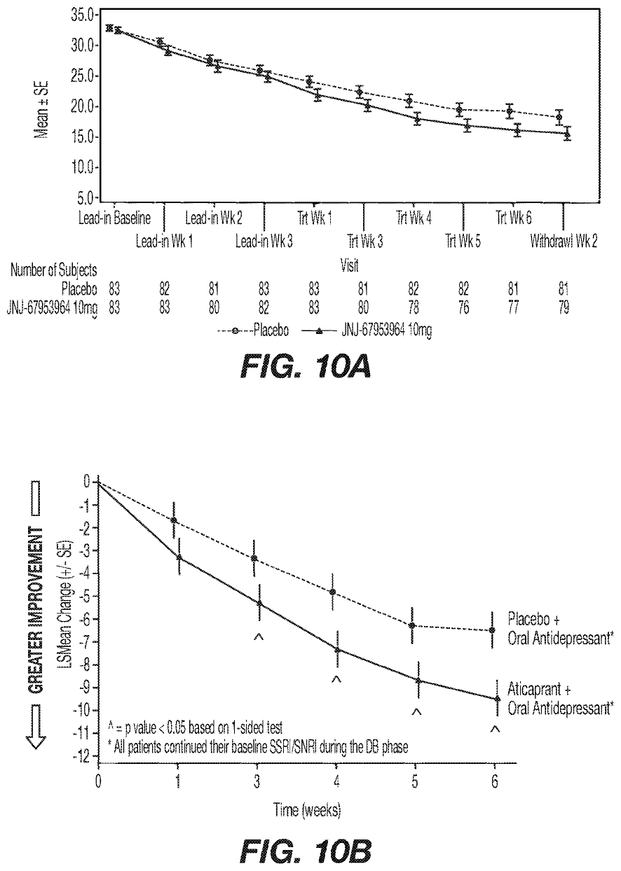

The mean (SD) baseline MADRS total score at treatment baseline was 25.3 (7.86), ranging from 0 to 41. See, FIGS. 10A and 10B. The mean changes from treatment baseline in MADRS total score at Treatment Week 6 for fITT were smaller than for eITT: −9.7 (8.02) for aticaprant and −6.6 (8.57) for placebo. The observed effect size was 0.36. These results illustrate a statistical superiority over placebo with a durability of effect with the greatest difference seen at week 6. See, Table 26.

TABLE 26
Summary of Baseline Psychiatry Rating Scales at the Start
of the Lead-in and Treatment Periods; fITT Analysis Set

| MADRS | | | SHAPS | | |
|---|---|---|---|---|---|
| Total Score | | | Total Score | | |
| | Mean | Median | | Mean | Median |
| N | (SD) | (Range) | N | (SD) | (Range) |

Lead-in Baseline

| | | | | | | |
|---|---|---|---|---|---|---|
| Placebo | 83 | 32.8 (4.25) | 33.0 (26; 42) | 83 | 37.8 (6.01) | 38.0 (22; 55) |
| Aticaprant | 83 | 32.4 (4.27) | 32.0 (21; 45) | 83 | 37.3 (6.23) | 38.0 (14; 53) |
| Total | 166 | 32.6 (4.25) | 32.0 (21; 45) | 166 | 37.6 (6.11) | 38.0 (14; 55) |

Treatment Baseline

| | | | | | | |
|---|---|---|---|---|---|---|
| Placebo | 83 | 25.7 (7.73) | 26.0 (10; 41) | 83 | 36.3 (5.44) | 36.0 (23; 50) |
| Aticaprant | 83 | 24.8 (8.02) | 27.0 (0; 36) | 83 | 35.0 (5.85) | 36.0 (14; 49) |
| Total | 166 | 25.3 (7.86) | 26.5 (0; 41) | 166 | 35.6 (5.67) | 36.0 (14; 50) |

Significant effect for aticaprant versus placebo in fITT population was also detected. The estimated LS mean difference at treatment week 6 between aticaprant and placebo was −3.1 with 80% 1-sided CI upper limit of −2.21. The corresponding p-value was 0.002. See, Tables 27-28 and FIG. 6.

TABLE 27
MADRS Total Score: MMRM Results - Estimated LS Means
and Comparison versus Placebo; fITT Analysis Set

| | | | Change from Baseline | | | | |
|---|---|---|---|---|---|---|---|
| | | | | | LSMean | 60% | |
| Analysis | | | | | Difference | Confidence | |
| Visit | | Mean | Mean | LSMean | (SE)\Treatment | Interval on | p- |
| Treatment | N | (SD) | (SD) | (SE) | Placebo | Difference | value^{a} |

Treatment Week 1

| | | | | | | | |
|---|---|---|---|---|---|---|---|
| Placebo | 83 | 24.0 | −1.8 | −1.7 | | | |
| | | (8.12) | (4.00) | (0.78) | | | |
| aticaprant | 83 | 21.7 | −3.1 | −3.2 | −1.6 | [−2.44, | 0.0653 |
| | | (8.78) | (4.81) | (0.77) | (1.03) | −0.70] | |

Treatment Week 3

| | | | | | | | |
|---|---|---|---|---|---|---|---|
| Placebo | 81 | 22.2 | −3.4 | −3.4 | | | |
| | | (9.28) | (6.50) | (0.78) | | | |
| aticaprant | 80 | 20.0 | −5.1 | −5.2 | −1.9 | [−2.74, | 0.0368 |
| | | (8.53) | (6.74) | (0.78) | (1.04) | −0.99] | |

Treatment Week 4

| | | | | | | | |
|---|---|---|---|---|---|---|---|
| Placebo | 82 | 20.8 | −4.9 | −4.8 | | | |
| | | (9.24) | (7.02) | (0.78) | | | |
| aticaprant | 78 | 17.9 | −7.2 | −7.3 | −2.5 | [−3.34, | 0.0093 |
| | | (9.32) | (7.02) | (0.78) | (1.04) | −1.59] | |

Treatment Week 5

| | | | | | | | |
|---|---|---|---|---|---|---|---|
| Placebo | 82 | 19.2 | −6.4 | −6.3 | | | |
| | | (9.89) | (7.16) | (0.78) | | | |
| aticaprant | 76 | 16.7 | −8.3 | −8.7 | −2.4 | [−3.24, | 0.0125 |
| | | (9.47) | (7.48) | (0.78) | (1.05) | −1.47] | |

Treatment Week 6

| | | | | | | | |
|---|---|---|---|---|---|---|---|
| Placebo | 81 | 19.0 | −6.6 | −6.5 | | | |
| | | (10.35) | (8.57) | (0.78) | | | |
| aticaprant | 77 | 15.9 | −9.7 | −9.6 | −3.1 | [−3.97, | 0.0017 |
| | | (9.09) | (8.02) | (0.79) | (1.05) | −2.21] | |

^{a}One-sided test for no difference between treatments from a MMRM model with subject as random effect; country, treatment, time and time-by-treatment interaction as factors; and baseline MADRS total score as continuous covariate. An AR(1) variance-covariance matrix was employed

TABLE 28
MADRS (Montgomery-Åsberg Depression Rating
Scale) Total Score: Mean Changes to Placebo
During the Treatment Period; fITT Analysis Set

| | | | | |
|---|---|---|---|---|
| | | Mean | 90% CI | |
| | | Change to | for Mean | |
| Analysis Visit | | Placebo | Change to | Effect |
| Treatment | N | (SD pooled) | Placebo | Size |

Treatment Week 1

| | | | | |
|---|---|---|---|---|
| Placebo | 83 | | | |
| Aticaprant | 83 | −1.3 (4.43) | [−2.4, −0.2] | −0.29 |

Treatment Week 3

| | | | | |
|---|---|---|---|---|
| Placebo | 81 | | | |
| Aticaprant | 80 | −1.7 (6.62) | [−3.4, 0.0] | −0.26 |

Treatment Week 4

| | | | | |
|---|---|---|---|---|
| Placebo | 82 | | | |
| Aticaprant | 78 | −2.3 (7.02) | [−4.1, −0.4] | −0.32 |

Treatment Week 5

| | | | | |
|---|---|---|---|---|
| Placebo | 82 | | | |
| Aticaprant | 76 | −1.9 (7.31) | [−3.9, −0.0] | −0.26 |

Treatment Week 6

| | | | | |
|---|---|---|---|---|
| Placebo | 81 | | | |
| Aticaprant | 77 | −3.0 (8.31) | [−5.2, −0.8] | −0.36 |

Negative change from baseline indicates improvement. Negative change to Placebo indicates favorable aticaprant effect. Negative effect size favors aticaprant; positive effect size favors Placebo.

### Covid-19 Impact on Primary Efficacy Assessment

Supplementary analysis was conducted using the same MMRM model as described for the primary analysis on all the data collected prior to 15 Mar. 2020 (estimated date of the COVID-19 lockdowns in most of the countries participating in the trial). Seventeen percent of the subjects in fITT and 19% in eITT population had at least one of the MADRS assessment excluded from the model due to COVID-19 impact. Results of the analysis corroborated the findings of the primary efficacy analysis in both: eITT and fITT populations. LSMeans difference estimate was −3.0 (80% 1-sided CI upper limit of −1.88) for eITT and −3.4 (80% 1-sided CI upper limit of −2.51) for fITT.

### (ii) Secondary Endpoints

### MADRS Remission Rates Over Treatment Period

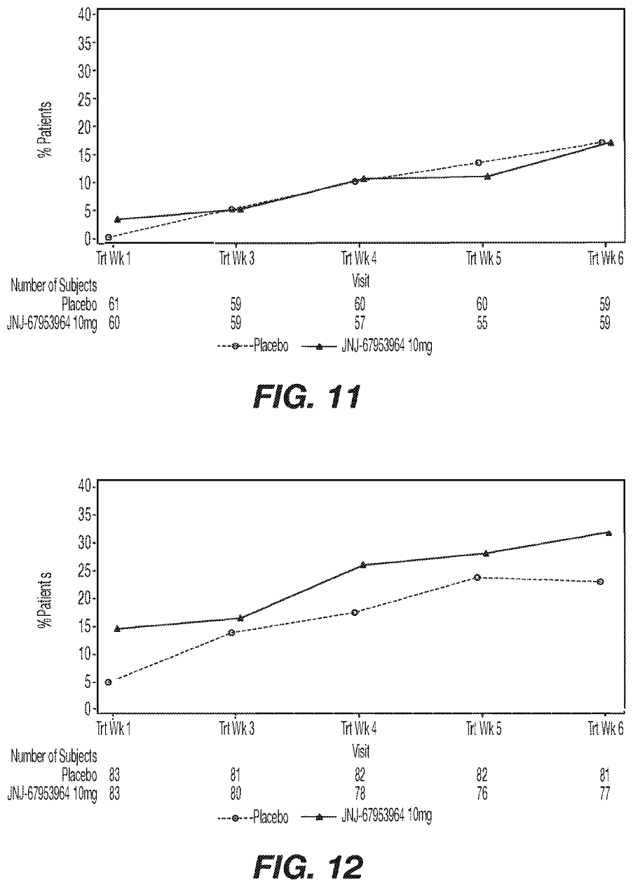

At Treatment Week 6 the percentage of subjects with MADRS remission (MADRS total score ≤10) in the eITT population was 16.9% for aticaprant and 16.9% for placebo. Treatment week 6 remission rates in fITT population were 31.2% for aticaprant and 22.2% for placebo. For both populations (eITT and fITT), no significant treatment differences were detected at treatment week 6 using Chi-square test (2-sided p=0.999 and p=0.203, respectively). See, FIGS. 11 and 12.

### MADRS Response Rates (at Least 30% Improvement) Over Treatment Period

The percentage of subjects with ≥30% improvement in MADRS total score at treatment week 6 in the eITT population was 57.6% for aticaprant and 45.8% for placebo. Treatment week 6 response rates in fITT population were 61.8% for aticaprant and for 44.4% placebo. For both populations, treatment differences at Treatment Week 6 were significant at 20% 2-sided significance level (Chi-square test: p=0.197 for eITT and p=0.029 for fITT).

### MADRS Response Rates (at Least 50% Improvement) Over Treatment Period

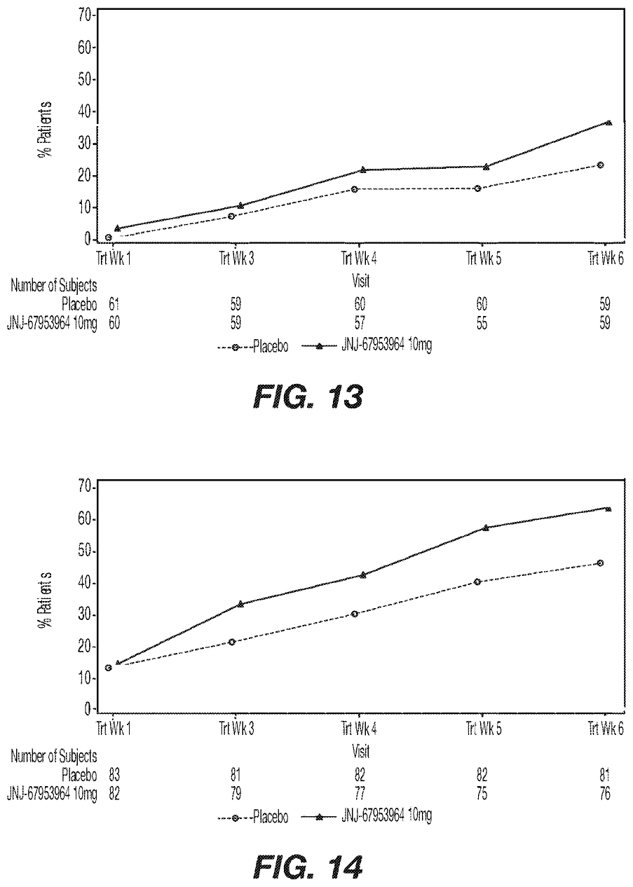

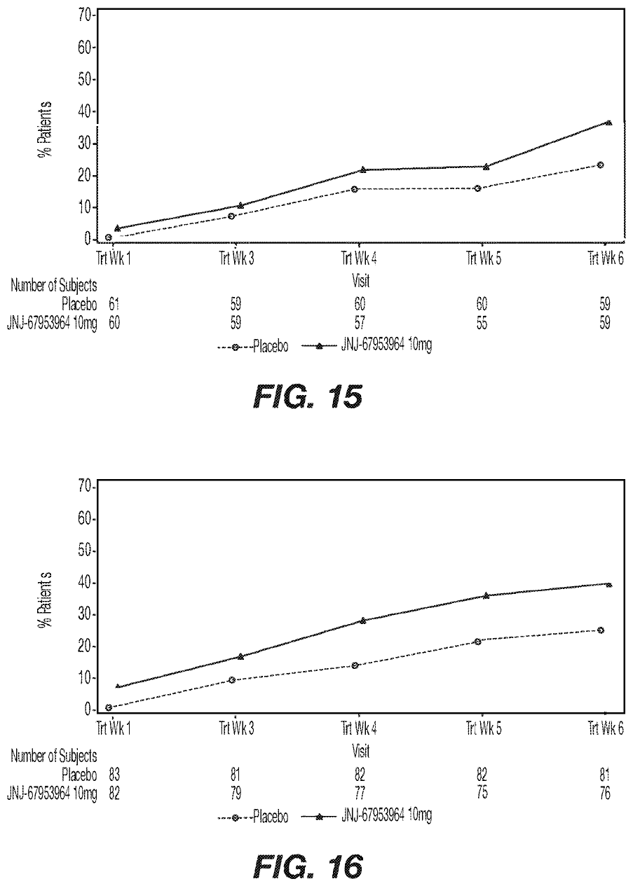

The percentage of subjects with ≥50% improvement in MADRS total score at treatment week 6 in the eITT population was 35.6% for aticaprant and 22.0% for placebo. Treatment week 6 response rates in fITT population were 38.2% for Aticaprant and 23.5% for placebo. For both populations, treatment differences at treatment week 6 were significant at 20% 2-sided significance level (Chi-square test: p=0.104 for eITT and p=0.046 for fITT). See, Table 29 and FIGS. 13-16.

TABLE 29
Change from Treatment Baseline in MADRS Total
Score at Treatment Week 6 in Both Responders
and Non-Responders during Placebo Lead-in Period

| | | aticaprant 10 |
|---|---|---|
| End point values | Placebo | milligrams (mg) |
| Number of subjects analyzed | 81 | 77 |
| Units: score on a scale | | |
| Measure Type: Least | −6.5 ± 0.78 | −9.6 ± 0.79 |
| Squares Mean (Standard | | |
| Error) | | |

| | |
|---|---|
| P-value | =0.0017 |
| Parameter type | Least Squares Mean Difference |
| Point estimate | −3.1 |
| Confidence interval | |
| level | 80% |
| sides | 1-Sided |
| lower limit | — |
| upper limit | −2.21 |
| Variability estimate | Standard Error of the mean |
| Dispersion value | 1.05 |

Changes in SHAPS Total Score from Treatment Baseline to Treatment Week 6

### Enriched ITT Analysis Set

In eITT population, in a subgroup of subjects with high anhedonia level (baseline SHAPS total score ≥38), larger differences between aticaprant placebo at Treatment Week 6 were observed than in subjects with low anhedonia level (20≤baseline SHAPS total score <38). The effect size was 0.38 and 0.11, respectively.

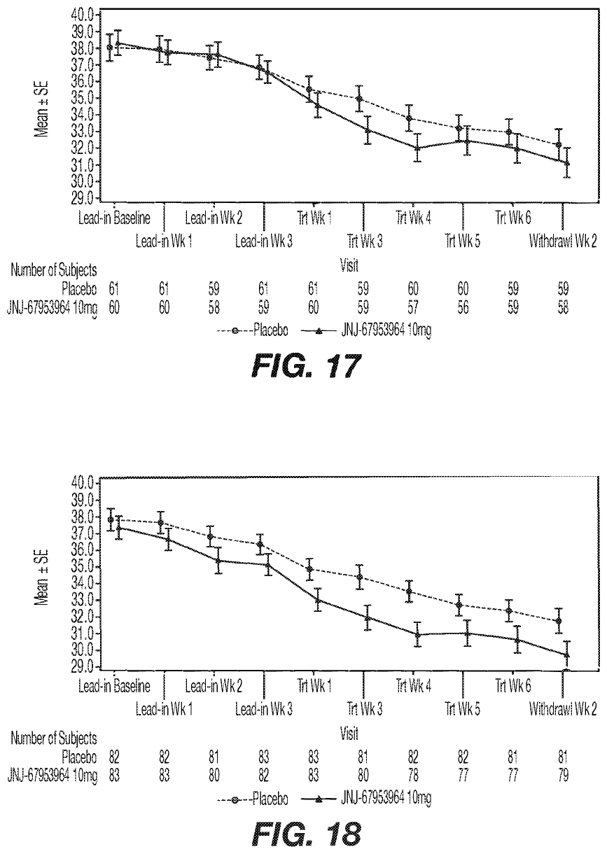

The mean (SD) SHAPS total score at treatment baseline was 36.6 (5.45), ranging from 20 to 50. The mean change from treatment baseline (SD) in SHAPS total score at treatment week 6 was −4.6 (6.23) for aticaprant and −4.2 (5.04) for placebo. The observed effect size was 0.07. See, Table 30 and FIGS. 17 and 25.

TABLE 30
SHAPS Total Score: Mean Changes to Placebo During
the Treatment Period; eITT Analysis Set

| | | | | | |
|---|---|---|---|---|---|
| | | Mean | Mean | 90% CI | |
| | | Change from | Change to | for Mean | |
| Analysis Visit | | Baseline | Placebo | Change to | Effect |
| Treatment | N | (SD) | (SD pooled) | Placebo | Size |

Treatment Week 1

| | | | | | |
|---|---|---|---|---|---|
| Placebo | 61 | −1.3 (3.17) | | | |
| aticaprant | 60 | −1.9 (4.30) | −0.6 (3.77) | [−1.7, 0.6] | −0.15 |

Treatment Week 3

| | | | | | |
|---|---|---|---|---|---|
| Placebo | 59 | −2.2 (4.65) | | | |
| aticaprant | 59 | −3.4 (5.25) | −1.2 (4.96) | [−2.8, 0.3] | −0.25 |

Treatment Week 4

| | | | | | |
|---|---|---|---|---|---|
| Placebo | 60 | −3.3 (4.47) | | | |
| aticaprant | 57 | −4.5 (5.89) | −1.2 (5.21) | [−2.8, 0.4] | −0.23 |

Treatment Week 5

| | | | | | |
|---|---|---|---|---|---|
| Placebo | 60 | −3.9 (4.88) | | | |
| aticaprant | 56 | −4.3 (6.07) | −0.4 (5.49) | [−2.1, 1.3] | −0.08 |

Treatment Week 6

| | | | | | |
|---|---|---|---|---|---|
| Placebo | 59 | −4.2 (5.04) | | | |
| aticaprant | 59 | −4.6 (6.23) | −0.4 (5.66) | [−2.1, 1.3] | −0.07 |

Negative change from baseline indicates improvement. Negative change to Placebo indicates favorable aticaprant effect. Negative effect size favors aticaprant; positive effect size favors Placebo.

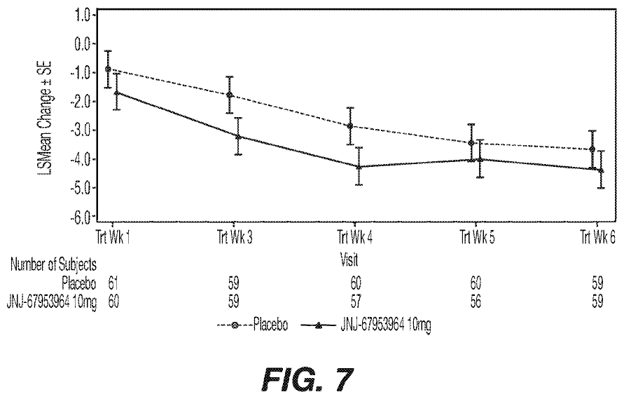

Changes in SHAPS total score were analyzed with the same MMRM model used for MADRS total score. The estimated LS Mean difference with 80% 2-sided CI at treatment week 6 between aticaprant and placebo was −0.7 [−1.81, 0.41]. See, FIG. 7 and Tables 31 and 32 and FIG. 18. The corresponding p-value was 0.419.

TABLE 31
SHAPS Total Score: MMRM Results - Estimated LS Means
and Comparison versus Placebo; eITT Analysis Set

| | | | Change from Baseline | | | | |
|---|---|---|---|---|---|---|---|
| | | | | | LSMean | 60% | |
| | | | | | Difference | Confidence | |
| Analysis Visit | | Mean | Mean | LSMean | (SE)\Treatment | Interval on | p- |
| Treatment | N | (SD) | (SD) | (SE) | Placebo | Difference | value^{a} |

Treatment Week 1

| | | | | | | | |
|---|---|---|---|---|---|---|---|
| Placebo | 61 | 35.5 | −1.3 | −0.9 | | | |
| | | (6.00) | (3.17) | (0.63) | | | |
| aticaprant | 60 | 34.5 | −1.9 | −1.7 | −0.8 | [−1.90, | 0.3542 |
| | | (5.63) | (4.30) | (0.64) | (0.86) | 0.31] | |

Treatment Week 3

| | | | | | | | |
|---|---|---|---|---|---|---|---|
| Placebo | 59 | 34.9 | −2.2 | −1.8 | | | |
| | | (6.09) | (4.65) | (0.64) | | | |
| aticaprant | 59 | 33.0 | −3.4 | −3.2 | −1.4 | [−2.53, | 0.1005 |
| | | (6.39) | (5.25) | (0.64) | (0.86) | −0.31] | |

Treatment Week 4

| | | | | | | | |
|---|---|---|---|---|---|---|---|
| Placebo | 60 | 33.7 | −3.3 | −2.9 | | | |
| | | (5.89) | (4.47) | (0.63) | | | |
| aticaprant | 57 | 32.0 | −4.5 | −4.3 | −1.4 | [−2.48, | 0.1131 |
| | | (6.24) | (5.89) | (0.64) | (0.86) | −0.26] | |

Treatment Week 5

| | | | | | | | |
|---|---|---|---|---|---|---|---|
| Placebo | 60 | 33.1 | −3.9 | −3.5 | | | |
| | | (5.88) | (4.88) | (0.64) | | | |
| aticaprant | 56 | 32.4 | −4.3 | −4.0 | −0.5 | [−1.65, | 0.5332 |
| | | (6.61) | (6.07) | (0.64) | (0.87) | 0.57] | |

Treatment Week 6

| | | | | | | | |
|---|---|---|---|---|---|---|---|
| Placebo | 59 | 32.9 | −4.2 | −3.7 | | | |
| | | (6.04) | (5.04) | (0.64) | | | |
| aticaprant | 59 | 31.9 | −4.6 | −4.4 | −0.7 | [−1.81, | 0.4188 |
| | | (6.60) | (6.23) | (0.64) | (0.87) | 0.41] | |

^{a}two-sided test for no difference between treatments from a MMRM model with subject as random effect; country, treatment, time and time-by-treatment interaction as factors; and baseline SHAPS total score as continuous covariate. An AR(1) variance-covariance matrix was employed.

TABLE 32
SHAPS Total Score: MMRM Results - Estimated LS Means
and Comparison versus Placebo; fITT Analysis Set

| | | | Change from Baseline | | | | |
|---|---|---|---|---|---|---|---|
| | | | | | LSMean | 60% | |
| Analysis | | | | | Difference | Confidence | |
| Visit | | Mean | Mean | LSMean | SE)\Treatment | Interval on | p- |
| Treatment | N | (SD) | (SD) | (SE) | Placebo | Difference | value^{a} |

Treatment Week 1

| | | | | | | | |
|---|---|---|---|---|---|---|---|
| Placebo | 83 | 34.8 | −1.5 | −1.0 | | | |
| | | (5.86) | (3.57) | (0.54) | | | |
| aticaprant | 83 | 32.9 | −2.0 | −1.9 | −1.0 | [−1.88, | 0.1888 |
| | | (6.09) | (4.05) | (0.54) | (0.72) | −0.02] | |

Treatment Week 3

| | | | | | | | |
|---|---|---|---|---|---|---|---|
| Placebo | 81 | 34.3 | −2.2 | −1.7 | | | |
| | | (6.36) | (5.11) | (0.54) | | | |
| aticaprant | 80 | 31.9 | −3.2 | −3.1 | −1.4 | [−2.32, | 0.0580 |
| | | (6.54) | (5.07) | (0.54) | (0.73) | −0.45] | |

Treatment Week 4

| | | | | | | | |
|---|---|---|---|---|---|---|---|
| Placebo | 82 | 33.4 | −3.0 | −2.5 | | | |
| | | (5.70) | (4.41) | (0.54) | | | |
| aticaprant | 78 | 30.8 | −4.2 | −4.1 | −1.6 | [−2.51, | 0.0321 |
| | | (6.37) | (5.70) | (0.55) | (0.73) | −0.63] | |

Treatment Week 5

| | | | | | | | |
|---|---|---|---|---|---|---|---|
| Placebo | 82 | 32.6 | −3.8 | −3.3 | | | |
| | | (5.63) | (4.76) | (0.55) | | | |
| aticaprant | 77 | 30.9 | −4.3 | −4.1 | −0.8 | [−1.71, | 0.2912 |
| | | (6.76) | (5.70) | (0.55) | (0.73) | 0.17] | |

Treatment Week 6

| | | | | | | | |
|---|---|---|---|---|---|---|---|
| Placebo | 81 | 32.2 | −4.2 | −3.7 | | | |
| | | (5.81) | (4.98) | (0.55) | | | |
| aticaprant | 77 | 30.5 | −4.7 | −4.5 | −0.8 | [−1.79, | 0.2503 |
| | | (6.98) | (5.91) | (0.55) | (0.73) | 0.10] | |

^{a}two-sided test for no difference between treatments from a MMRM model with subject as random effect; country, treatment, time and time-by-treatment interaction as factors; and baseline SHAPS total score as continuous covariate. An AR(1) variance-covariance matrix was employed

The estimated LS mean differences with 80% 2-sided CI at treatment week 6 between aticaprant and placebo was −0.8 [−1.79, 0.10]. The corresponding p-value was 0.250. See, FIGS. 7 and 8.

### Full ITT Analysis Set

Similar trend was observed in fITT population and differences were larger in magnitude than those observed in eITT population. The effect size was 0.51 and 0.29, respectively. The mean (SD) baseline SHAPS total score at treatment baseline was 35.6 (5.67), ranging from 14 to 50. The mean changes from treatment baseline in SHAPS total score at treatment week 6 for fITT population were similar to changes in eITT: −4.7 (5.91) for aticaprant and −4.2 (4.98) for placebo. The observed effect size was 0.08. See, Table 33.

TABLE 33
SHAPS Total Score: Mean Changes to Placebo During
the Treatment Period; fITT Analysis Set

| | | | | | |
|---|---|---|---|---|---|
| | | Mean | Mean | 90% CI for | |
| | | Change from | Change to | Mean | |
| Analysis Visit | | Baseline | Placebo | Change to | Effect |
| Treatment | N | (SD) | (SD pooled) | Placebo | Size |

Treatment Week 1

| | | | | | |
|---|---|---|---|---|---|
| Placebo | 83 | −1.5 (3.57) | | | |
| aticaprant | 83 | −2.0 (4.05) | −0.6 (3.82) | [−1.5, 0.4] | −0.15 |

Treatment Week 3

| | | | | | |
|---|---|---|---|---|---|
| Placebo | 81 | −2.2 (5.11) | | | |
| aticaprant | 80 | −3.2 (5.07) | −1.0 (5.09) | [−2.4, 0.3] | −0.20 |

Treatment Week 4

| | | | | | |
|---|---|---|---|---|---|
| Placebo | 82 | −3.0 (4.41) | | | |
| aticaprant | 78 | −4.2 (5.70) | −1.2 (5.08) | [−2.5, 0.1] | −0.23 |

Treatment Week 5

| | | | | | |
|---|---|---|---|---|---|
| Placebo | 82 | −3.8 (4.76) | | | |
| aticaprant | 77 | −4.3 (5.70) | −0.5 (5.24) | [−1.8, 0.9] | −0.09 |

Treatment Week 6

| | | | | | |
|---|---|---|---|---|---|
| Placebo | 81 | −4.2 (4.98) | | | |
| aticaprant | 77 | −4.7 (5.91) | −0.5 (5.45) | [−1.9, 1.0] | −0.08 |

Negative change from baseline indicates improvement. Negative change to Placebo indicates favorable aticaprant effect. Negative effect size favors aticaprant; positive effect size favors Placebo.

Changes in MADRS Total Score from Treatment Baseline to Treatment Week 6 by Anhedonia Level at Baseline

### Enriched ITT Analysis Set

In subgroup of subjects with high anhedonia level (SHAPS total score ≥38) at treatment baseline, n=53, larger differences between aticaprant and placebo at treatment Week 6 were observed than in subjects with low anhedonia level (20≤baseline SHAPS total score <38), n=65: −3.4 with 90% 2-sided CI of [−7.5, 0.7] and −0.9 with 90% 2-sided CI of [−4.2, 2.5], respectively (Table 34). The observed effect size was 0.38 and 0.11, respectively.

TABLE 34
MADRS (Montgomery-Åsberg Depression Rating Scale) Total
Score: Mean Changes to Placebo During the Treatment Period
by Anhedonia Level at Treatment Baseline; eITT Analysis Set

| | | | | | |
|---|---|---|---|---|---|
| | | Mean | Mean | 90% CI | |
| Analysis | | Change from | Change to | for Mean | |
| Visit | | Baseline | Placebo | Change to | Effect |
| Treatment | N | (SD) | (SD pooled) | Placebo | size |

Low anhedonia
Treatment Week 1

| | | | | | |
|---|---|---|---|---|---|
| Placebo | 34 | −1.8 (3.43) | | | |
| aticaprant | 34 | −2.3 (5.03) | −0.5 (4.30) | [−2.2, 1.2] | −0.12 |

Treatment Week 3

| | | | | | |
|---|---|---|---|---|---|
| Placebo | 32 | −4.8 (5.70) | | | |
| aticaprant | 33 | −4.9 (5.99) | −0.1 (5.85) | [−2.5, 2.4] | −0.01 |

Treatment Week 4

| | | | | | |
|---|---|---|---|---|---|
| Placebo | 33 | −6.5 (6.16) | | | |
| aticaprant | 32 | −6.4 (7.40) | 0.0 (6.80) | [−2.8, 2.9] | 0.01 |

Treatment Week 5

| | | | | | |
|---|---|---|---|---|---|
| Placebo | 33 | −7.6 (6.80) | | | |
| aticaprant | 29 | −7.2 (6.46) | 0.3 (6.65) | [−2.5, 3.2] | 0.05 |

Treatment Week 6

| | | | | | |
|---|---|---|---|---|---|
| Placebo | 32 | −8.3 (8.25) | | | |
| aticaprant | 33 | −9.2 (8.01) | −0.9 (8.13) | [−4.2, 2.5] | −0.11 |

High anhedonia
Treatment Week 1

| | | | | | |
|---|---|---|---|---|---|
| Placebo | 27 | −2.7 (4.08) | | | |
| aticaprant | 26 | −4.6 (5.25) | −1.8 (4.69) | [−4.0, 0.3] | −0.39 |

Treatment Week 3

| | | | | | |
|---|---|---|---|---|---|
| Placebo | 27 | −3.6 (6.35) | | | |
| aticaprant | 26 | −6.7 (6.83) | −3.0 (6.59) | [−6.1, 0.0] | −0.46 |

Treatment Week 4

| | | | | | |
|---|---|---|---|---|---|
| Placebo | 27 | −6.3 (7.34) | | | |
| aticaprant | 25 | −8.5 (7.26) | −2.2 (7.30) | [−5.6, 1.2] | −0.30 |

Treatment Week 5

| | | | | | |
|---|---|---|---|---|---|
| Placebo | 27 | −7.1 (7.67) | | | |
| aticaprant | 26 | −9.7 (8.18) | −2.6 (7.93) | [−6.3, 1.0] | −0.33 |

Treatment Week 6

| | | | | | |
|---|---|---|---|---|---|
| Placebo | 27 | −8.1 (9.01) | | | |
| aticaprant | 26 | −11.5 (8.95) | −3.4 (8.98) | [−7.5, 0.7] | −0.38 |

Low Anhedonia level (SHAPS Total Score at Treatment Baseline >=20 and <38), High Anhedonia level (SHAPS Total Score at Treatment Baseline >=38). The MADRS Total Score ranges from 0 to 60, with higher scores indicating greater severity of depression.

### Full ITT Analysis Set

A similar trend was observed in fITT population. The differences were larger in magnitude compared to eITT population: −4.6 with 90% 2-sided CI of [−8.4, −0.8] for subjects with high anhedonia level (n=63) and −2.3 with 90% 2-sided CI of [−5.0, 0.4] for subjects with low anhedonia level (n=94). See, Table 35. The observed effect size was 0.51 and 0.29, respectively.

TABLE 35
MADRS (Montgomery-Åsberg Depression Rating Scale) Total
Score: Mean Changes to Placebo During the Treatment Period
by Anhedonia Level at Treatment Baseline; fITT Analysis Set

| | | | | | |
|---|---|---|---|---|---|
| | | Mean | Mean | 90% CI | |
| Analysis | | Change from | Change to | for Mean | |
| Visit | | Baseline | Placebo | Change to | Effect |
| Treatment | N | (SD) | (SD pooled) | Placebo | size |

Low anhedonia
Treatment Week 1

| | | | | | | |
|---|---|---|---|---|---|---|
| Placebo | 49 | −1.3 | (4.17) | | | |
| aticaprant | 52 | −2.4 | (4.59) | −1.0 (4.39) | [−2.5, 0.4] | −0.24 |

Treatment Week 3

| | | | | | | |
|---|---|---|---|---|---|---|
| Placebo | 47 | −3.6 | (6.04) | | | |
| aticaprant | 49 | −4.1 | (6.67) | −0.5 (6.37) | [−2.7, 1.7] | −0.08 |

Treatment Week 4

| | | | | | | |
|---|---|---|---|---|---|---|
| Placebo | 48 | −4.9 | (6.53) | | | |
| aticaprant | 48 | −6.4 | (6.77) | −1.5 (6.65) | [−3.8, 0.8] | −0.23 |

Treatment Week 5

| | | | | | | |
|---|---|---|---|---|---|---|
| Placebo | 48 | −6.6 | (6.82) | | | |
| aticaprant | 45 | −7.3 | (6.90) | −0.7 (6.86) | [−3.1, 1.7] | −0.10 |

Treatment Week 6

| | | | | | | |
|---|---|---|---|---|---|---|
| Placebo | 47 | −6.5 | (8.11) | | | |
| aticaprant | 47 | −8.8 | (7.48) | −2.3 (7.80) | [−5.0, 0.4] | −0.29 |

High anhedonia
Treatment Week 1

| | | | | | | |
|---|---|---|---|---|---|---|
| Placebo | 34 | −2.4 | (3.71) | | | |
| aticaprant | 30 | −4.4 | (5.04) | −2.0 (4.38) | [−3.8, −0.1] | −0.45 |

Treatment Week 3

| | | | | | | |
|---|---|---|---|---|---|---|
| Placebo | 34 | −3.1 | (7.17) | | | |
| aticaprant | 30 | −6.9 | (6.66) | −3.8 (6.94) | [−6.7, −0.9] | −0.54 |

Treatment Week 4

| | | | | | | |
|---|---|---|---|---|---|---|
| Placebo | 34 | −4.8 | (7.75) | | | |
| aticaprant | 29 | −8.6 | (7.32) | −3.8 (7.56) | [−7.0, −0.6] | −0.50 |

Treatment Week 5

| | | | | | | |
|---|---|---|---|---|---|---|
| Placebo | 34 | −6.2 | (7.72) | | | |
| aticaprant | 30 | −10.2 | (8.04) | −4.0 (7.87) | [−7.3, −0.7] | −0.51 |

Treatment Week 6

| | | | | | | |
|---|---|---|---|---|---|---|
| Placebo | 34 | −6.8 | (9.30) | | | |
| aticaprant | 29 | −11.3 | (8.69) | −4.6 (9.03) | [−8.4, −0.8] | −0.51 |

Low Anhedonia level (SHAPS Total Score at Treatment Baseline >=20 and <38), High Anhedonia level (SHAPS Total Score at Treatment Baseline >=38). The MADRS Total Score ranges from 0 to 60, with higher scores indicating greater severity of depression.

This data illustrates that segmentation into high vs low anhedonia had a benefit for treating MDD: higher treatment effect for Aticaprant. Further, the placebo response was lower in patients with high anhedonia, as compared to low anhedonia.

Change from Treatment Baseline in CGI-S Total Score at Treatment

TABLE 36
Change from Treatment Baseline in
CGI-S Total Score at Treatment

| | | aticaprant 10 |
|---|---|---|
| End point values | Placebo | milligrams (mg) |
| Number of subjects analyzed | 59 | 59 |

Units: Scores on a scale

| | | |
|---|---|---|
| Measure Type: Arithmetic | −0.76 ± 0.858 | −0.92 ± 1.039 |
| Mean (SD) | | |

Change from Treatment Baseline in SMDDS Total Score at Treatment Week 6

TABLE 37
Change from Treatment Baseline in
SMDDS Total Score at Treatment Week

| | | aticaprant 10 |
|---|---|---|
| End point values | Placebo | milligrams (mg) |
| Number of subjects analyzed | 59 | 59 |

Units: Scores on a scale

| | | |
|---|---|---|
| Measure Type: Arithmetic | −8.49 ± 9.567 | −8.03 ± 9.957 |
| Mean (SD) | | |

Number of Subjects with SATE Score at Treatment Week 6

TABLE 38
Number of Subjects with SATE Score at Treatment Week 6

| | | aticaprant 10 |
|---|---|---|
| | | milligrams |
| End point values | Placebo | (mg) |
| Number of subjects analyzed | 61 | 60 |

Units: subjects

| | | |
|---|---|---|
| Overall Depression (Got worse) | 1 | 0 |
| (n = 40, 30) | | |
| Overall Depression (Not changed) | 12 | 9 |
| (n = 40, 30) | | |
| Overall Depression (Improved) | 27 | 21 |
| (n = 40, 30) | | |
| Depression Worsened (Slightly worse) | 1 | 0 |
| (n = 1, 0) | | |
| Depression Worsened (Much worse) | 0 | 0 |
| (n = 1, 0) | | |
| Depression Worsened (Very much worse) | 0 | 0 |
| (n = 1, 0) | | |
| Depression Slightly improved | 13 | 15 |
| (n = 27, 21) | | |
| Depression Much improved | 11 | 6 |
| (n = 27, 21) | | |
| Depression Very Much Improved | 3 | 0 |
| (n = 27, 21) | | |

Change from Treatment Baseline in HAM-A6 Total Score at Treatment Week 6

TABLE 39
Change from Treatment Baseline in HAM-A6
Total Score at Treatment Week 6

| | | aticaprant 10 |
|---|---|---|
| End point values | Placebo | milligrams (mg) |
| Number of subjects analyzed | 59 | 59 |

Units: scores on a scale

| | | |
|---|---|---|
| Measure Type: Arithmetic | −2.19 ± 2.837 | −2.73 ± 2.651 |
| Mean (SD) | | |

These data show a greater improvement in HAMA6 score in aticaprant treated patients vs. placebo.

Change from Treatment Baseline in Structured Interview Guide for the SIGH-A Score at Treatment Week 6

TABLE 40
Change from Treatment Baseline in Structured Interview
Guide for the SIGH-A Score at Treatment Week 6

| | | aticaprant 10 |
|---|---|---|
| End point values | Placebo | milligrams (mg) |
| Number of subjects analyzed | 59 | 59 |

Units: scores on a scale

| | | |
|---|---|---|
| Measure Type: Arithmetic | −5.37 ± 6.549 | −5.85 ± 5.369 |
| Mean (SD) | | |

### Maximum Plasma Concentration (Cₘₐₓ) of Aticaprant

Cₘₐₓ is defined as maximum plasma concentration of aticaprant. The eITT population included all enrolled lead-in placebo non-responders who were randomized into a treatment period, received at least 1 dose of study medication, and had at least 1 post-baseline MADRS assessment during the treatment period. Here ‘N’ (number of subjects analyzed) includes the number of subjects evaluable for this endpoint. Here ‘n’ (number analyzed) included all subjects evaluable for specified time point categories.

TABLE 41
Cₘₐₓ of Aticaprant (10 mg)

| | |
|---|---|
| Number of subjects analyzed | 58 |

Units: nanograms per milliliter (ng/mL)

| | |
|---|---|
| Measure Type: Arithmetic | |
| Mean (SD) | |
| Week 1 (n = 56) | 32.7 ± 10.9 |
| Week 3 (n = 56) | 33.5 ± 11.1 |
| Week 6 (n = 56) | 34.3 ± 11.1 |

No statistical analyses of this end point.

(iii) Safety Endpoints

Overall, in full safety analysis set 40/85 (47.1%) of subjects in the aticaprant group and 30/84 (35.7%) of subjects in the placebo group experienced at least one TEAE during the treatment period. See, Table 42.

TABLE 42
Overall Summary of Treatment-Emergent Adverse Events
During the Treatment Period; Full Safety Analysis Set

| | Placebo | | aticaprant | | Overall |
|---|---|---|---|---|---|
| | (N = 84) | | 10 mg (N = 85) | | (N = 169) |
| | n (%) | | n (%) | | n (%) |
| Subjects with 1 or more | 30 | (35.7) | 40 | (47.1) | 70 (41.4) |
| TEAE | | | | | |
| Total subjects affected by | 9 | (10.7%) | 23 | (27.1%) | |
| non-serious adverse events | | | | | |
| Subjects with drug-related | 13 | (15.5) | 20 | (23.5) | 33 (19.5) |
| TEAE ^{a} | | | | | |
| Subjects with TEAE leading | 0 | | 0 | | 0 |
| to death | | | | | |
| Subjects with 1 or more | 1 | (1.2) | 0 | | 1 (0.6) |
| serious TEAE | | | | | |
| Subjects with TEAE leading | 1 | (1.2) | 1 | (1.2) | 2 (1.2) |
| to discontinuation of agent | | | | | |

^{a} Drug relationships of possible, probable, and very likely are included in this category.
Subjects are presented by the treatment received during the Treatment period.

The most common TEAEs during the treatment period were headache (experienced by 10/85 subjects-11.8% in the aticaprant group and by 6/84 subjects-7.1% in the placebo group) and diarrhea (experienced by 7/85 subjects-8.2% in the aticaprant group and by 2/84 subjects-2.4% in the placebo group). See, Table 43.

TABLE 43
Treatment-Emergent Adverse Events by Body System
or Organ Class and Dictionary-Derived Term in >=
5% of Subjects in Either Treatment Group During
the Treatment Period; Full Safety Analysis Set

| | | aticaprant | |
|---|---|---|---|
| | Placebo | 10 mg | Overall |
| Body System | (N = 84) | (N = 85) | (N = 169) |
| Preferred Term | n (%) | n (%) | n (%) |
| Total no. Subjects with | 30 (36) | 40 (47) | 70 (41) |
| Adverse Events | | | |
| Infections And Infestations | 9 (11) | 13 (15) | 22 (13) |
| Nasopharyngitis | 2 (2) | 5 (6) | 7 (4) |
| Nervous System Disorders | 9 (11) | 13 (15) | 22 (13) |
| Headache | 6 (7) | 10 (12) | 16 (10) |
| Gastrointestinal Disorders | 9 (11) | 12 (14) | 21 (12) |
| Diarrhea | 2 (2) | 7 (8) | 9 (5) |
| Skin And Subcutaneous | 3 (4) | 6 (7) | 9 (5) |
| Tissue Disorders | | | |
| Pruritus | 0 | 5 (6) | 5 (3) |

Percentages calculated with the number of subjects in each group as denominator. Reported dictionary version: MedDRA 22.1. Subjects are presented by the treatment received during the Treatment period.

There were 2 subjects in total who discontinued during the treatment period due to treatment-emergent adverse events: 1 subject in the aticaprant 10 group due to diarrhea, nausea, vomiting and headache, and another subject in placebo group due to acute calculous cholecystitis.

Overall, 17/169 subjects experienced TEAEs of special interest during the treatment period: 13/85 (15.3%) in the aticaprant group and 4/84 (4.8%) in the placebo group. The most common treatment-emergent adverse events during the treatment phase were headache and diarrhea. The most common TEAE of special interest during the treatment period were diarrhea and pruritus (experienced by 5/85 subjects-5.9% in the aticaprant group and by 0/84 subjects in the placebo group). Further 1 patient in the placebo group (1.19%) experienced acute cholecystitis, as compared to 0 patients receiving aticaprant. See, Table 44.

TABLE 44
Treatment-Emergent Adverse Events of Special Interest
During the Treatment Period; Full Safety Analysis Set

| | | aticaprant | |
|---|---|---|---|
| | Placebo | 10 mg | Overall |
| Body System | (N = 84) | (N = 85) | (N = 169) |
| Preferred Term | n (%) | n (%) | n (%) |
| Total no. Subjects with | 4 (4.8) | 13 (15.3) | 17 (10.1) |
| Adverse Events of Special | | | |
| Interest | | | |
| Gastrointestinal Disorders | 4 (4.8) | 9 (10.6) | 13 (7.7) |
| deaths causally related | | | |
| to treatment/all | | | |
| Diarrhea | 2 (2.4) | 7 (8.2) | 9 (5.3) |
| Abdominal Pain Upper | 2 (2.4) | 0 | 2 (1.2) |
| Dyspepsia | 1 (1.2) | 1 (1.2) | 2 (1.2) |
| Abdominal Pain | 0 | 1 (1.2) | 1 (0.6) |
| Skin And Subcutaneous | 0 | 5 (5.9) | 5 (3.0) |
| Tissue Disorders | | | |
| Pruritus | 0 | 5 (5.9) | 5 (3.0) |

Percentages calculated with the number of subjects in each group as denominator. Reported dictionary version: MedDRA 22.1. Subjects are presented by the treatment received during the Treatment period.

Two serious adverse events occurred. One subject in the placebo group experienced acute calculous cholecystitis during the treatment period and other subject suicidal ideation during the lead-in period. Both subjects discontinued due to these AEs.

No deaths were reported.

### (iv) Anhedonia Analysis

Patients in the larger fITT group maintained baseline level of depression and anhedonia severity consistent with the eITT group. See, Tables 45-47.

TABLE 45
Frequency of Subjects with Anhedonia at
Treatment Baseline; fITT Analysis Set

| | | | No Anhedonia | Anhedonia |
|---|---|---|---|---|
| | | | (SHAPS Total | (SHAPS Total |
| | | N | Score < 20) | Score >= 20) |
| Baseline/Day 22 | | | | |
| | Placebo | 83 | 0 | 83 (100%) |
| | aticaprant | 83 | 1 (1.2%) | 82 (98.8%) |
| | Total | 166 | 1 (0.6%) | 165 (99.4%) |

Anhedonia classification is based on calculated SHAPS total score at Visit Day 22

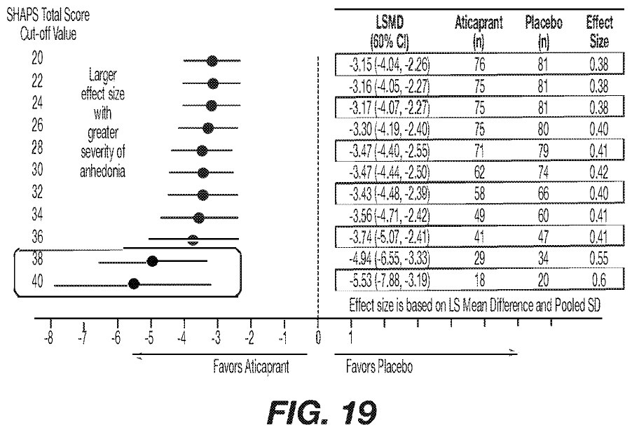

The results illustrate that treatment effect is larger in patients with more anhedonia at baseline. See, FIG. 19.

TABLE 46
Frequency of Subjects with Different Level of Anhedonia at
Treatment Baseline and Treatment Week 6; eITT Analysis Set

| | | | |
|---|---|---|---|
| | | Low Level | High Level |
| | No Anhedonia | of Anhedonia | of Anhedonia |
| | (SHAPS Total | (20 <= SHAPS | (SHAPS Total |
| N | Score < 20) | Total Score < 38) | Score >= 38) |

Treatment Baseline

| | | | | |
|---|---|---|---|---|
| Placebo | 61 | 0 | 34 (55.74%) | 27 (44.26%) |
| aticaprant | 60 | 0 | 34 (56.67%) | 26 (43.33%) |
| Total | 121 | 0 | 68 (56.2%) | 53 (43.8%) |

Treatment Week 6

| | | | | |
|---|---|---|---|---|
| Placebo | 59 | 0 | 46 (77.97%) | 13 (22.03%) |
| aticaprant | 59 | 3 (5.08%) | 48 (81.36%) | 8 (13.56%) |
| Total | 118 | 3 (2.54%) | 94 (79.66%) | 21 (17.8%) |

Anhedonia classification is based on re-calculated SHAPS total score at analysis visits Treatment Baseline and Treatment Week 6.

TABLE 47
Frequency of Subjects with Different Level of Anhedonia at
Treatment Baseline and Treatment Week 6; fITT Analysis Set

| | | | |
|---|---|---|---|
| | | Low Level of | High Level of |
| | No Anhedonia | Anhedonia | Anhedonia |
| | (SHAPS Total | (20 >= SHAPS | (SHAPS Total |
| N | Score < 20) | Total Score < 38) | Score >= 38) |

Treatment Baseline

| | | | | |
|---|---|---|---|---|
| Placebo | 83 | 0 | 49 (59.04%) | 34 (40.96%) |
| aticaprant | 83 | 1 (1.2%) | 52 (62.65%) | 30 (36.14%) |
| Total | 166 | 1 (0.6%) | 101 (60.84%) | 64 (38.55%) |

Treatment Week 6

| | | | | |
|---|---|---|---|---|
| Placebo | 81 | 0 | 66 (81.48%) | 15 (18.52%) |
| aticaprant | 77 | 7 (9.09%) | 62 (80.52%) | 8 (10.39%) |
| Total | 158 | 7 (4.43%) | 128 (81.01%) | 23 (14.56%) |

Anhedonia classification is based on re-calculated SHAPS total score at analysis visits Treatment Baseline and Treatment Week 6.

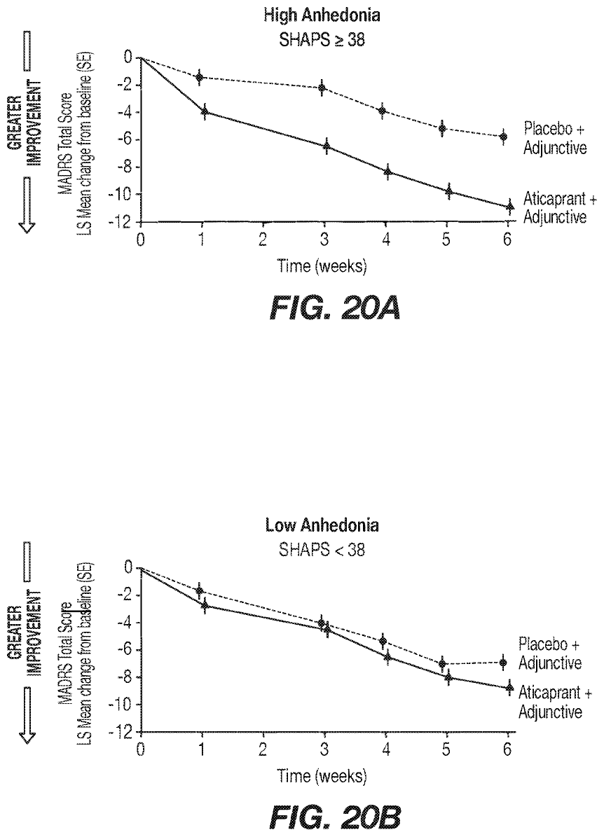

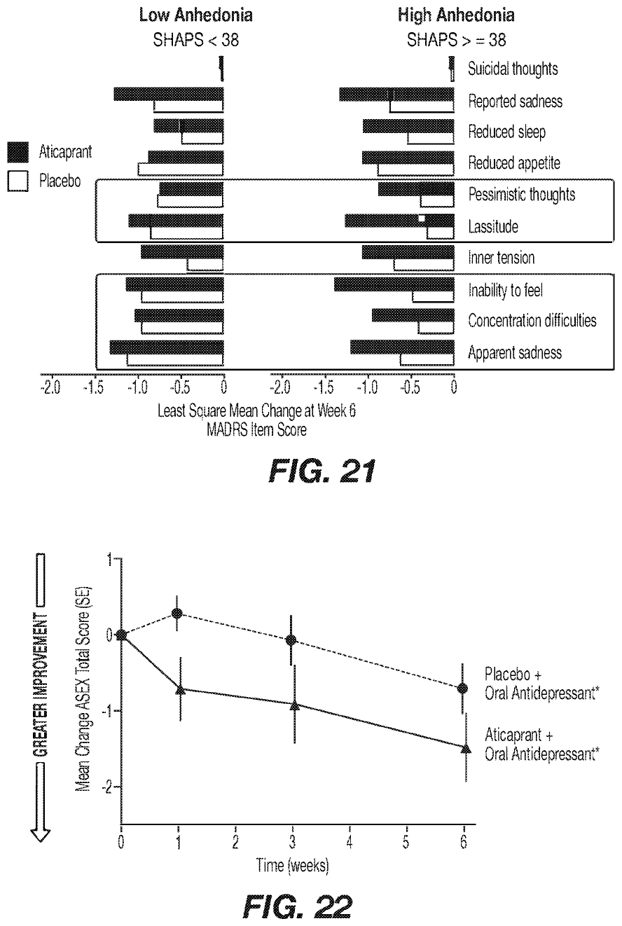

The results illustrate that the treatment effect is larger in patients with more anhedonia at baseline. See, FIGS. 20-A and 20-B. In FIG. 20-A, i.e., the high anhedonia group, the placebo+oral antidepressant group shows less placebo response as compared to the low anhedonia group in FIGS. 7-8. Similarly the treatment effect of the aticaprant+oral antidepressant group is higher in the high anhedonia group as compared to the low anhedonia group. Overall the effect size is larger at every single time point (from week 1 onwards) in the high anhedonia group. The LSMD in the high anhedonia group is more than double that of the low anhedonia group at week 6. Further, when looking at the symptom level, greater improvement in items related to anhedonia and dysphoria in subgroup with high anhedonia vs low anhedonia. See, FIG. 21.

### (v) Weight Change

At the lead-in baseline timepoint, the mean weight for subjects in the placebo group was 76.17 kg compared to 78.66 in the aticaprant group. After 6 weeks in the double-blind treatment phase, the mean weight in the placebo group was 75.75 kg compared to 78.57 kg in the aticaprant group. This indicates that the weight in both groups remained relatively stable over the 6-week double blind treatment period. This is unexpected because other adjunctive treatments for MDD result in a mean weight increase. See, Thase M, et al. J Clin Psych. 2015: 76 (9), 1224-1231; Thase, J Clin Psych. 2015, 76 (9): 1232-1240; El Khalili, Int J Neuropsychopharmacol. 2010, 13, 917-932; Marcus, J. Clin. Psychopharmacol. 2008, 28:156-165; Berman, J. Clin. Psychiatry 2007; 68:843-853; Berman, American College of Neuropsychopharmacology, 2008, Annual Meeting Abstracts (Scottsdale, Ariz, Dec. 7-11, 2008). Nashville, Tenn, ACNP, 2008; Earley, American College of Neuropsychopharmacology, 2007, Annual Meeting Abstracts (Boca Raton, Fla, Dec. 9-13, 2007). Nashville, TN, ACNP, 2007). See, Table 48.

TABLE 48
Mean weight by treatment group (kg)

| | Placebo | Aticaprant |
|---|---|---|
| | n = 84 | n = 85 |
| Screening, mean (SE) | 76.39 (1.61) | 78.42 (1.65) |
| Lead-in Baseline, mean (SE) | 76.17 (1.61) | 78.66 (1.65) |
| Withdrawal Baseline, mean (SE) | 75.75 (1.62) | 78.57 (1.71) |
| Absolute Change | −0.42 | −0.09 |
| (Withdrawal − Lead-in) | | |
| Relative % Change | −0.55% | −0.11% |

### (vi) Completion Rate

Patients who passed the screening phase entered a lead in phase followed by a double-blind phase. Patients who responded to placebo during the lead in phase were labelled as non-responders. Patients who did not respond to placebo were labelled as non-responders. The double-blind treatment phase then continued for an additional 6 weeks, after which patients entered a withdrawal period.

Of the 121 subjects in the enriched population (60 in aticaprant and 61 in placebo group), 117 (96.7%) completed the study. The overall completion rate for the full ITT analysis set is 95%. This contrasts with completion rates of approximately 85% for studies of adjunctive aripiprazole (Pae, CNS Drugs, 2011; 25, 109-127) and 45-62% for adjunctive quetiapine (El Khalili cited above). In total 4 subjects (3.3%) discontinued the study: 2 subjects in placebo and 2 subjects in aticaprant treatment group. See, Tables 49 and 50.

TABLE 49
Completion/Early Withdrawal Information; eITT Analysis Set

| | | |
|---|---|---|
| | aticaprant | |
| Placebo | 10 mg | Total |
| (N = 61) | (N = 60) | (N = 121) |

Subject Completed Treatment/Trial

| | | | |
|---|---|---|---|
| Completed | 59 (96.7%) | 58 (96.7%) | 117 (96.7%) |
| Withdrawn | 2 (3.3%) | 2 (3.3%) | 4 (3.3%) |

Reason For Withdrawal/Termination

| | | | |
|---|---|---|---|
| Lack of Efficacy | 0 | 1 (1.7%) | 1 (0.8%) |
| Non-compliance with drug | 0 | 1 (1.7%) | 1 (0.8%) |
| Withdrawal by subject | 1 (1.6%) | 0 | 1 (0.8%) |
| Other | 1 (1.6%) | 0 | 1 (0.8%) |

Percentages calculated with the number of subjects in each group as denominator.

TABLE 50
Completion/Early Withdrawal Information;
Full Safety Analysis Set

| | | |
|---|---|---|
| | aticaprant | |
| Placebo | 10 mg | Total |
| (N = 84) | (N = 85) | (N = 169) |

Subject Completed Treatment/Trial

| | | | |
|---|---|---|---|
| Completed | 81 (96.4%) | 79 (92.9%) | 160 (94.7%) |
| Withdrawn | 3 (3.6%) | 6 (7.1%) | 9 (5.3%) |

Reason For Withdrawal/Termination

| | | | |
|---|---|---|---|
| Adverse event | 1 (1.2%) | 1 (1.2%) | 2 (1.2%) |
| Lack of Efficacy | 0 | 2 (2.4%) | 2 (1.2%) |
| Non-compliance with drug | 0 | 1 (1.2%) | 1 (0.6%) |
| Protocol deviation | 0 | 1 (1.2%) | 1 (0.6%) |
| Withdrawal by subject | 1 (1.2%) | 0 | 1 (0.6%) |
| Other | 1 (1.2%) | 1 (1.2%) | 2 (1.2%) |

Percentages calculated with the number of subjects in each group as denominator.

(vii) Sexual Functioning

Impairments in sexual functioning is a common side effect of antidepressant treatment and can be very upsetting to patients and their sexual partners. Major depression itself is associated with increased sexual dysfunction, and many of the pharmacological treatments are known to worsen sexual functioning even further. In a large survey of nearly 5000 patients in France, it was estimated that in untreated patients with MDD, the prevalence of sexual dysfunction was 65%. The prevalence of sexual dysfunction increased to 71% for patients treated with antidepressant therapy.

Sexual pleasure is an important component of hedonic tone. The brain reward circuitry is controlled by several areas: nucleus accumbens, ventral tegmental area and the amygdala. It is hypothesized that treatment with kappa opioid receptors may restore the normal homeostatic balance in patients with overactivation. Treatment with aticaprant could potentially improve symptoms of anhedonia. Other symptoms associated with the reward circuitry includes: sexual pleasure, lack of interest and lack of enjoyment.

Patients had their sexual functioning measured using a standard, well accepted rating scale: ASEX. See, Table 51.

TABLE 51
ASEX scores by treatment group

| | Placebo | Aticaprant |
|---|---|---|
| | n = 84 | n = 85 |
| Baseline | 22.04 | 21.26 |
| Endpoint | 21.36 | 19.79 |
| Absolute Change | −0.68 | −1.47 |
| Relative % Change | −3.09% | −6.91% |

The mean change from treatment baseline (SD) in ASEX total score to week 6 was −1.5 (4.02) points for aticaprant compared to −0.7 (2.98) points for placebo. A lower score on the ASEX indicates improvement. The score reduction at week 6 was greater in the aticaprant group compared to placebo. This is unexpected because adjunctive treatments with other agents are expected to worsen sexual functioning, i.e., increase in ASEX score over time. See, FIG. 22.

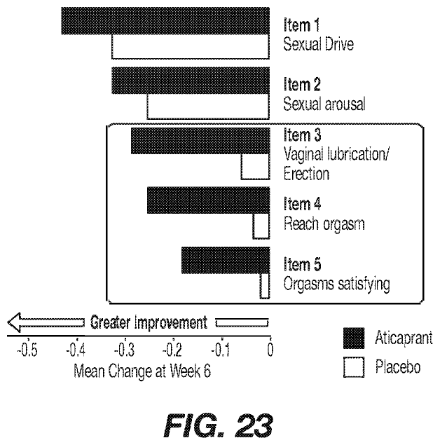

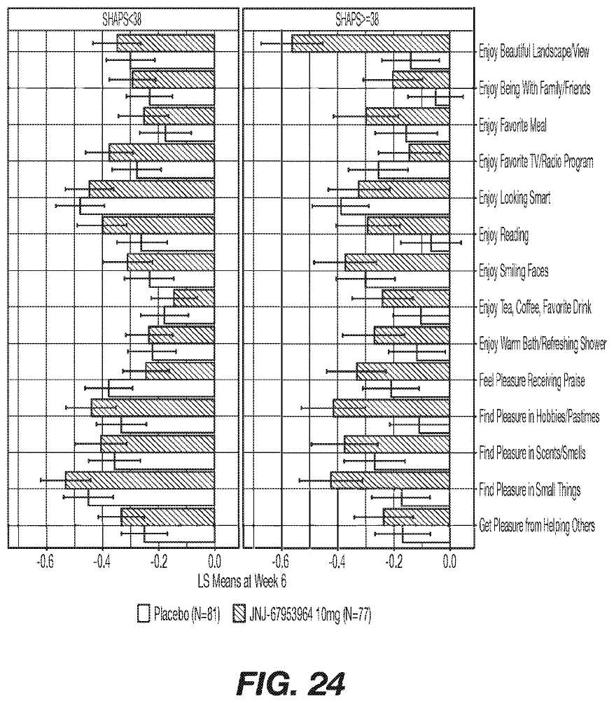

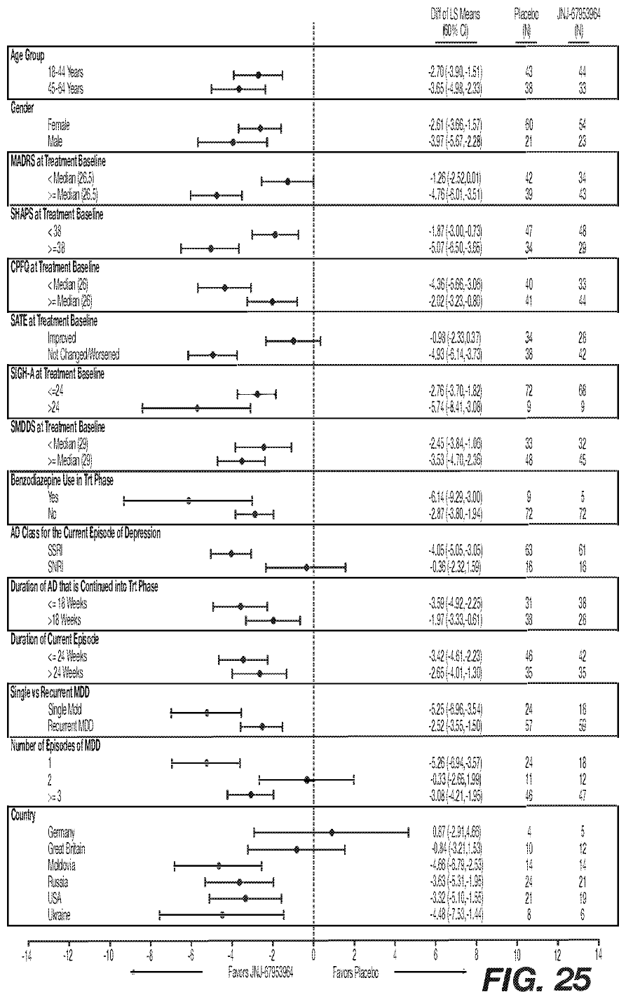

Patients receiving aticaprant had notable improvements in sexual functioning. An examination of individual item level changes was also conducted and revealed that the greatest changes were seen in items related to consummatory pleasure: orgasm satisfying, reach orgasm and vaginal lubrication/erection. Most of the improvements seen in items 3, 4 and 5 of FIG. 23.

(viii) Onset of Effect

The onset of effect for aticaprant can be estimated from the study. FIG. 10-B depicts the least squares mean change from baseline. A significant treatment effect favoring aticaprant was seen as early as week 3. At this point, aticaprant showed a statistically superior effect compared to placebo.

What is claimed is:

1. A composition comprising a crystalline form of aticaprant and less than about 0.05% by weight, based on the weight of the composition, of 3,4-bis(4-((2-(3,5-dimethylphenyl) pyrrolidin-1-yl)methyl) phenoxy)benzamide:

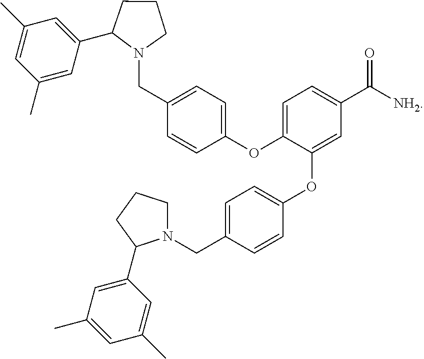 [SMILES: Cc1cc(C)cc(C2CCCN2Cc2ccc(Oc3ccc(C(N)=O)cc3Oc3ccc(CN4CCCC4c4cc(C)cc(C)c4)cc3)cc2)c1]

2. The composition of claim 1, wherein the crystalline form of aticaprant is characterized by four or more x-ray diffraction pattern peaks at 2θ (±0.2) of 4.1°, 9.0°, 17.6°, 18.0°, or 21.4°.

3. The composition of claim 1, wherein the crystalline form of aticaprant is characterized by a differential scanning calorimetry peak temperature (Tₘ) at about 121° C.

4. The composition of claim 1, wherein the crystalline form of aticaprant is characterized by an x-ray powder diffraction pattern that corresponds to FIG. 1 and/or a differential scanning calorimetry thermogram that corresponds to FIG. 4.

5. The composition of claim 1, wherein the crystalline form of aticaprant is anhydrous.

6. The composition of claim 1, comprising about 0.001 to about 0.05% by weight, based on the weight of the composition, of 3,4-bis(4-((2-(3,5-dimethylphenyl) pyrrolidin-1-yl)methyl)phenoxy)benzamide.

7. The composition of claim 1, comprising about 0.10% by weight or less, based on the weight of the composition, of 3-fluoro-4-(4-formylphenoxy)benzamide:

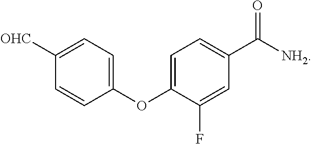 [SMILES: NC(=O)c1ccc(Oc2ccc(C=O)cc2)c(F)c1]

8. The composition of claim 7, comprising about 0.001 to about 0.1% by weight, based on the weight of the composition, of 3-fluoro-4-(4-formylphenoxy)benzamide

9. The composition of claim 1, comprising at least about 99.5% by weight, based on the weight of the composition, of the crystalline form of aticaprant.

10. The composition of claim 1, comprising about 0.10% by weight or less, based on the weight of the composition, of R-aticaprant:

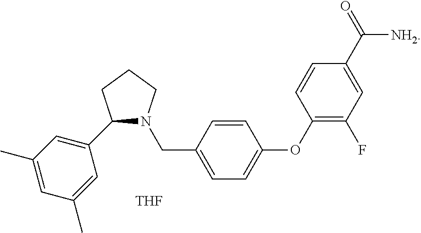 [SMILES: "THF" ; Cc1cc(C)cc([C@H]2CCCN2Cc2ccc(Oc3ccc(C(N)=O)cc3F)cc2)c1]

11. The composition of claim 10, comprising about 0.001 to about 0.1% by weight, based on the weight of the composition, of R-aticaprant.

12. The composition of claim 1, comprising about 2 to about 35 mg, about 5 mg to about 10 mg, about 5 mg, or about 10 mg aticaprant.

13. A method of treating major depressive disorder in a human patient, the method comprising administering the composition of claim 1 to the human patient.

14. The method of claim 13, wherein the human patient has anhedonia.

15. The method of claim 13, wherein the human patient had a previous inadequate response to other antidepressant therapy.

16. The method of claim 15, wherein the other antidepressant therapy is a selective serotonin reuptake inhibitor.

17. The method of claim 15, wherein the other antidepressant therapy is a serotonin-norepinephrine reuptake inhibitor.

18. The method of claim 15, wherein the other antidepressant therapy is a selective serotonin reuptake inhibitor and a serotonin-norepinephrine reuptake inhibitor.

19. The method of claim 15, further comprising adjunctive treatment with an effective amount of one or more antidepressants.

20. The method of claim 19, wherein the one or more antidepressants is a selective serotonin reuptake inhibitor.

21. The method of claim 19, wherein the one or more antidepressants is a serotonin-norepinephrine reuptake inhibitor.

22. The method of claim 19, wherein the one or more antidepressants is a selective serotonin reuptake inhibitor and a serotonin-norepinephrine reuptake inhibitor.

23. The method of claim 15, wherein the composition comprises about 2 to about 35 mg, about 5 mg to about 10 mg, about 5 mg, or about 10 mg aticaprant.

24. The method of claim 13, wherein the composition comprises about 10 mg of S-aticaprant and is administered orally and once daily as an adjunctive therapy to a selective serotonin reuptake inhibitor (SSRI), wherein the patient had an inadequate response to at least one antidepressant comprising a selective serotonin reuptake inhibitor (SSRI) prior to treatment with the aticaprant.
